(19) United States
(12) Patent Application Publication
(10) Pub. No.: US 2014/0066439 A1
(43) Pub. Date: Mar. 6, 2014

(54) OSTEOCLAST ACTIVITY

(71) Applicants: Jan Gunst, Ieper (BE); Helen Owen, Heverlee (BE); Greet Van den Berghe, Grez-Doiceau (BE); Ilse Vanhorebeek, Lubbeek (BE)

(72) Inventors: Jan Gunst, Ieper (BE); Helen Owen, Heverlee (BE); Greet Van den Berghe, Grez-Doiceau (BE); Ilse Vanhorebeek, Lubbeek (BE)

(73) Assignee: Katholieke Universiteit Leuen, K.U.Leuven R&D, Leunven (BE)

(21) Appl. No.: 13/884,913

(22) PCT Filed: Nov. 10, 2011
(86) PCT No.: PCT/BE11/00064

§ 371 Date: Jul. 29, 2013

Related U.S. Application Data
(60) Provisional application No. 61/458,930, filed on Dec. 2, 2010.

(30) Foreign Application Priority Data
Nov. 10, 2010 (GB) 1019013.0
Nov. 23, 2010 (GB) 1019882.8
Dec. 1, 2010 (GB) 1020377.6
Apr. 1, 2011 (GB) 1105578.7
Apr. 1, 2011 (GB) 1105579.5
Apr. 1, 2011 (GB) 1105580.3

(51) Int. Cl. A61K 31/5415; A61K 31/436; A61K 45/06; A61K 31/132

(52) U.S. Cl. CPC A61K 31/5415; A61K 31/132; A61K 31/436; A61K 45/06
USPC 514/226.2; 514/674; 514/291; 514/224.8

(57) ABSTRACT

Described are medicaments and methods of treating or preventing metabolic bone diseases, such as Critical Illness Related Metabolic Bone Disease or of critical illness induced Osteopenia secondary to ICU Admission by sufficient autophagy inducing compound to inhibit or suppress critical illness enhanced osteoclastogenesis or increased osteoclast differentiation. The methods include administering of an autophagy activating compound to a mammal to: treat or prevent a bone degenerative disorder; slow bone deterioration; restore lost bone; maintain bone mass and/or bone quality or inhibit bone resorption in particularly by inhibiting or reducing a process by which osteoclasts break down bone and release the minerals resulting in a transfer of calcium from bone fluid to the blood. Also described are methods for administering the autophagy activating compound to treat a bone disorder of hyperresorption of bone and/or enhanced activation of osteoclasts.

### CROSS-REFERENCE TO RELATED APPLICATION

This application is a national phase entry under 35 U.S.C. §371 of International Patent Application PCT/BE2011/000064, filed Nov. 10, 2011, designating the United States of America and published in English as International Patent Publication WO2012/061907 A2 on May 18, 2012, which claims the benefit under Article 8 of the Patent Cooperation Treaty to United Kingdom Application Serial Nos. 1019013.0, filed Nov. 10, 2010, 1019882.8, filed Nov. 23, 2010, 1020377.6, filed Dec. 1, 2010, 1105580.3, filed Apr. 1, 2011, 1105579.5, filed Apr. 1, 2011, 1105578.7, filed Apr. 1, 2011, and under Article 8 of the Patent Cooperation Treaty and 35 U.S.C. §119(e) to U.S. Ser. No. 61/458,930, filed Dec. 2, 2010.

### TECHNICAL FIELD

The disclosure relates to medicaments and novel methods of treating metabolic bone diseases, and more particularly for treating or preventing Critical Illness Related Metabolic Bone Disease or of critical illness induced Osteopenia [ICD-10 M85.8, ICD-9 733.90, DiseasesDB 29870 or MeSH D001851] secondary to ICU Admission by sufficient autophagy inducing compound to inhibit or suppressing critical illness [MeSH Descriptor: C23.550.291.625] enhanced osteoclastogenesis or increased osteoclast differentiation. The disclosed therapeutic methods include administering of an autophagy activating compound to a mammal to: (1) treat or prevent a bone degenerative disorder; (2) slow bone deterioration; (3) restore lost bone; (4) maintain bone mass and/or bone quality or (5) inhibit bone resorption in particularly by inhibiting or reducing a process by which osteoclasts break down bone and release the minerals resulting in a transfer of calcium from bone fluid to the blood. Also provided are methods for administering the autophagy activating compound to treat a bone disorder of hyperresorption of bone and/or enhanced activation of osteoclasts.

Several documents are cited throughout the text of this specification. Each of the documents herein (including any manufacturer's specifications, instructions etc.) are hereby incorporated by reference; however, there is no admission that any document cited is indeed prior art of the invention.

### BACKGROUND

A number of conditions are associated with a loss of bone, particularly in the elderly and/or postmenopausal women. For example, osteoporosis is a debilitating disease characterized by a decrease in skeletal bone mass and mineral density, structural deterioration of the bone, and corresponding increases in bone fragility and susceptibility to fracture. Osteoporosis in humans is preceded by clinical osteopenia, a condition found in approximately 25 million people in the United States only.

Throughout adult life, bone continually undergoes a turnover through the coupled processes of bone formation and resorption. Bone resorption is mediated by bone resorbing cells, osteoclasts, which are formed by mononuclear phagocytic cells. New bone replacing the lost bone is deposited by bone-forming cells, osteoblasts, which are formed by mesenchymal stromal cells. Various other cell types that participate in the remodeling process are tightly controlled by systemic factors (e.g., hormones, lymphokines, growth factors, and vitamins) and local factors (e.g., cytokines, adhesion molecules, lymphokines, and growth factors). The proper spatiotemporal coordination of the bone remodeling process is essential to the maintenance of bone mass and integrity. A number of bone degenerative disorders are linked to an imbalance in the bone remodeling cycle which results in abnormal loss of bone mass (osteopenia) including metabolic bone diseases, such as, osteoporosis, osteoplasia (osteomalacia) and osteodystrophy.

Critically ill patients have an increased osteoclast formation from circulating precursors in the blood, osteoclast maturation and osteoclast bone resorption activity in comparison to healthy patients.

There are currently two main types of pharmaceutical therapy available for the treatment of osteoporosis. The first, and most common, approach is the use of hormone therapy to reduce the resorption of bone tissue. Estrogen replacement therapy (“ERT”) is known to prevent further deterioration and thus reduce the likelihood of fractures. However, the use of estrogen as a treatment is limited, as it is believed that long-term estrogen therapy may be associated with risk of uterine cancer, endometrial cancer, breast cancer, frequent vaginal bleeding, and thrombosis. Because of these serious side effects, many women choose to avoid this treatment. Further, few men agree to this type of therapy. The second major therapeutic approach to osteoporosis is the use of bisphosphonates, particularly alendronate, risedronate, and ibandronate. Although tests have shown that these compounds consistently increase the bone mineral density in osteoporosis patients, there are also significant problems with the treatment of osteoporosis by bisphosphonates, including irritation of the oesophagus and upper gastrointestinal tract.

Therefore, there exists a need to develop new therapeutic methods for treating and preventing bone disorders.

### SUMMARY OF THE DISCLOSURE

Disclosed are solutions to the problems associated with metabolic bone diseases.

Disclosed are medicaments and novel methods for treating or preventing bone degenerative disorders. The disorders treated or prevented include, for example, osteopenia, osteomalacia, osteoporosis, osteomyeloma, osteodystrophy, osteogenesis imperfecta, and bone degenerative disorders associated with chronic renal disease, hyperparathyroidism, long-term use of corticosteroids and critical illness.

The disclosure includes reversing the decrease in bone formation markers (serum osteocalcin), and an increase in bone resorption markers (urinary pyridinoline) in a subject, for instance, a critically ill subject by spermidine or analogues thereof.

In one aspect, to treat with spermidine or analogues thereof a disorder of bone metabolism in a subject, for instance, to reverse a disorder of decrease in bone formation and an increase in bone resorption. Such disorder can be caused by critical illness.

Another aspect of the disclosure concerns treating with an autophagy activator or enhancer a disorder of bone metabolism in a subject, for instance, to reverse a disorder of decrease in bone formation and an increase in bone resorption. Such disorder can be caused by critical illness. Particular aspects and embodiments of the invention are the following. An autophagy activator or autophagy inducing compound for use as a medicine to inhibit increased bone resorption. An autophagy activator or autophagy inducing compound for use as a medicine to inhibit osteoclast activity. An autophagy activator or autophagy inducing compound for use as a medicine to inhibit the formation of osteoclast (TRAP positive multinuclear cells) from blood progenitor cells. An autophagy activator or autophagy inducing compound for use as a medicine to activate bone formation. An autophagy activator or autophagy inducing compound for use as a medicine to treat a bone degenerative disorder that is a bone metabolism disorder. An autophagy activator or autophagy inducing compound for use as a medicine to treat a bone degenerative disorder, which is caused by acute critical illness or prolonged critical illness. An autophagy activator or autophagy inducing compound for use as a medicine to treat a bone degenerative disorder which is caused by (parenteral) nutrition induced suppression of autophagy. An autophagy activator or autophagy inducing compound for use as a medicine to treat a bone degenerative disorder which is caused by mTOR activation. And/or an autophagy activator or autophagy inducing compound for use as a medicine to treat a bone degenerative disorder, which is hyperresorption of bone.

An embodiment concerns an autophagy activator or autophagy inducing compound for use as a medicine to treat a bone degenerative disorder that is pathological increased bone resorption and pathological decreased bone formation. An autophagy activator or autophagy inducing compound for use as a medicine to treat a bone degenerative disorder that is a disrupted calcium and bone metabolism by acute critical illness and/or prolonged critical illness. Another embodiment concerns an autophagy activator or autophagy inducing compound for use as a medicine to treat a bone degenerative disorder that is pathological increased bone resorption and pathological decreased bone formation. An autophagy activator or autophagy inducing compound for use as a medicine to treat a bone degenerative disorder caused by enhanced osteoclast formation from circular precursors in the blood during critical illness. Yet another embodiment concerns an autophagy activator or autophagy inducing compound for use as a medicine to treat a bone degenerative disorder that is pathological increased bone resorption and pathological decreased bone formation. An autophagy activator or autophagy inducing compound for use as a medicine to treat a bone degenerative disorder that is increased osteoclast formation from circulating precursor leading to osteoporosis.

The disclosure is predicated on the discovery by the inventors that the disorder of critical illness [MeSH Descriptor C23.550.291.625] correlates with increased osteoclast formation from circulating precursors in the blood, osteoclast maturation and osteoclast bone resorption activity in comparison to healthy patients, resulting in complications, such as, “Osteopenia” (ICD-10 M85.8, ICD-9 733.90, DiseasesDB 29870 or MeSH D001851) or leading to the indication Critical Illness Bone Atrophy, Metabolic Bone Disease Following ICU Admission, Critical Illness Related Metabolic Bone Disease, Osteoporosis Secondary to ICU Admission with more extreme bone loss which increases the risk of critically ill patients for fractures, such as, stress fractures (ICD-10-CM 733.93 Stress fracture of tibia or fibula, 733.94 Stress fracture of the metatarsals, 733.95 Stress fracture of other bone, 733.96 Stress fracture of femoral neck, 733.97 Stress fracture of shaft of femur and/or 733.98 Stress fracture of pelvis) and that usually needs that usually needs post-ICU ambulatory care. We found that trabecular bone mineral content and density were reduced in critically ill rabbits, coinciding with low serum levels of ionized calcium and osteocalcin. Moreover, it was found that increased number of PBMC osteoclast precursors in the blood of critically ill humans resulted in increased in vitro osteoclast formation, which was further potentiated by the addition of serum from critically ill patients. Unexpectedly, neutralizing the inflammatory cytokines TNF-α and IL-6 in patient serum further increased osteoclast formation in patient PBMC cultures.

Further scope of applicability of the disclosure will become apparent from the detailed description given hereinafter. However, it should be understood that the detailed description and specific examples are given by way of illustration only, since various changes and modifications within the spirit and scope of the disclosure will become apparent to those skilled in the art from this detailed description. It is to be understood that both the foregoing general description and the following detailed description are exemplary and explanatory only and are not restrictive, as claimed. Some embodiments are set forth in claim format directly below:

### DETAILED DESCRIPTION

Several documents are cited throughout the text of this specification. Each of the documents herein (including any manufacturer's specifications, instructions etc.) are hereby incorporated by reference; however, there is no admission that any document cited is indeed prior art to the disclosure.

The disclosure will be described with respect to particular embodiments and with reference to certain drawings but the invention is not limited thereto but only by the claims.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments described herein, are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention, described herein, are capable of operation in other orientations than described or illustrated herein.

It is to be noticed that the “comprising,” used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a composition comprising means A and B” should not be limited to the compositions consisting only of components A and B. It means that with respect to the invention, the only relevant components of the composition are A and B.

Reference throughout this specification to “one embodiment” or “an embodiment” means that a particular feature, structure or characteristic described in connection with the embodiment is included in at least one embodiment. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments, various features are sometimes grouped together in a single embodiment, figure, or description thereof for the purpose of streamlining the disclosure and aiding the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment.

Furthermore, while some embodiments, described herein, include some, but not other, features included in other embodiments, combinations of features of different embodiments are meant to be within the scope hereof, and form different embodiments, as would be understood by those in the art. For example, in the following claims, any of the claimed embodiments can be used in any combination.

In the description provided herein, numerous specific details are set forth. However, it is understood that embodiments may be practiced without these specific details. In other instances, well-known methods, structures and techniques have not been shown in detail in order not to obscure an understanding of this description.

Other embodiments will be apparent to those skilled in the art from consideration of the specification and practice of the disclosure.

It is intended that the specification and examples be considered as exemplary only.

Each and every claim is incorporated into the specification as an embodiment of the invention. Thus, the claims are part of the description and are a further description and are in addition to the preferred embodiments of the invention.

The following terms are provided solely to aid in the understanding of the disclosure.

An osteoclast is a type of bone cell that removes bone tissue by removing its mineralized matrix and breaking up the organic bone. For instance, organic dry weight is about 90% collagen. This process is known as bone resorption. Osteoclasts and osteoblasts are instrumental in controlling the amount of bone tissue: osteoblasts form bone, osteoclasts resorb bone. Osteoclasts are formed by the fusion of cells of the monocyte-macrophage cell line and osteoclasts can be characterized by high expression of tartrate resistant acid phosphatase (TRAP) and cathepsin K.

“Microtubule associated protein light chain) 3” (LC3) is an ubiquitin-like protein that binds to autophagosomes (AVs). Cellular biologists transfect mammalian cells with GFP tagged LC3 to track and follow the fate of AVs in the cell and to measure autophagic flux.

“Bone Remodeling” (or bone metabolism) is a life-long process where mature bone tissue is removed from the skeleton (a process called bone resorption) and new bone tissue is formed (a process called ossification or new bone formation). These processes also control the reshaping or replacement of bone during growth and following injuries like fractures but also micro-damage, which occurs during normal activity. Remodeling responds also to functional demands of the mechanical loading. As a result, bone is added where needed and removed where it is not required. It is a tightly regulated process involving the interaction of osteoblasts and osteoclasts

The term “pharmaceutically acceptable” is used adjectivally herein to mean that the compounds are appropriate for use in a pharmaceutical product. The term “physiologically acceptable” also means that the compounds are appropriate for use in a pharmaceutical product.

As used herein, an “autophagy activator” is any compound that increases autophagy within a cell. An increase in autophagy may be determined as known in the art and described herein. Exemplary, non-limiting autophagy activators are known in the art and include, for example, proteasome inhibitor, tamoxifen, trehalose, vinblastine, rapamycin, Azithromycin macrolide or its analogues, that inhibit the mammalian target of rapamycin (mTOR) (a negative regulator of autophagy), ganima-benzene hexachloride, or of a derivative thereof, which is obtainable by chemical substitution. Yet, azithromycin has retained the capacity of acting as an inducer or stimulator of autophagy maturation.

As used herein, the phrase “physiologically acceptable salts” or “pharmaceutically acceptable salts” or “nutraceutically acceptable salts” refers to salts prepared from pharmaceutically acceptable, preferably nontoxic, acids and bases, including inorganic and organic acids and bases, including but not limited to, sulfuric, citric, maleic, acetic, oxalic, hydrochloride, hydro bromide, hydro iodide, nitrate, sulfate, bisulfite, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, fornate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Pharmaceutically acceptable salts include those formed with free amino groups, such as, but not limited to, those derived from hydrochloric, phosphoric, acetic, oxalic, and tartaric acids. Pharmaceutically acceptable salts also include those formed with free carboxyl groups, such as, but not limited to, those derived from sodium, potassium, ammonium, sodium lithium, calcium, magnesium, ferric hydroxides, isopropylamine, triethylamine, 2-ethylamino ethanol, histidine, and procaine.

As used herein, the term “carrier” refers to a diluent, adjuvant, excipient, or vehicle. Such carriers can be sterile liquids, such as, saline solutions in water, or oils, including those of petroleum, animal, vegetable or synthetic origin, such as, peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions.

As used herein, the term “mineral” refers to a substance, preferably a natural substance that contains calcium, magnesium or phosphorus. Illustrative nutrients and minerals include beef bone, fish bone, calcium phosphate, egg shells, sea shells, oyster shells, calcium carbonate, calcium chloride, calcium lactate, calcium gluconate and calcium citrate.

The term “treatment” refers to any process, action, application, therapy, or the like, wherein a mammal, including a human being, is subject to medical aid with the object of improving the mammal's condition, directly or indirectly.

In its broadest sense, the term a “critically ill patient” (herein designated CIP) refers to a patient who is experiencing an acute life-threatening episode or who is diagnosed to be in imminent danger of such an episode. A critically ill patient is medically unstable, and when not treated, likely to die.

The term “critically ill” patient refers to a patient who has sustained or is at risk of sustaining acutely life-threatening single or multiple organ system failure due to disease or injury, a patient who is being operated and where complications supervene, and a patient who has been operated in a vital organ within the last week or has been subject to major surgery within the last week.

In a more restricted sense, the term a “critically ill patient,” as used herein, refers to a patient who has sustained or is at risk of sustaining acutely life-threatening single or multiple organ system failure due to disease or injury, or a patient who is being operated and where complications supervene.

In an even more restricted sense, the term a “critically ill patient,” as used herein, refers to a patient who has sustained or is at risk of sustaining acutely life-threatening single or multiple organ system failure due to disease or injury. Similarly, these definitions apply to similar expressions, such as, “critical illness in a patient” and a “patient is critically ill.” A critically ill patient is also a patient in need of cardiac surgery, cerebral surgery, thoracic surgery, abdominal surgery, vascular surgery, or transplantation, or a patient suffering from neurological diseases, cerebral trauma, respiratory insufficiency, abdominal peritonitis, multiple trauma, severe burns, or critical illness polyneuropathy.

The term “critical illness” as used herein, refers to the condition of a “critically ill patient.” ICU scoring systems, such as, APACHE II & III, Glasgow Coma Scale PIM2 SAPS II SAPS III SOFA define the measure the severity of critical illness disorder for adult patients admitted to intensive care units and predict hospital mortality risk for critically ill hospitalized adults.

The term “Intensive Care Unit” (herein designated ICU), as used herein, refers to the part of a hospital where critically ill patients are treated. Of course, this might vary from country to country and even from hospital to hospital and the part of the hospital may not necessary, officially, bear the name “Intensive Care Unit” or a translation or derivation thereof. Of course, the term “Intensive Care Unit” also covers a nursing home, a clinic, for example, a private clinic, or the like if the same or similar activities are performed there. The term “ICU patient” refers to a “critically ill patient.”

The term “parenterally administering” refers to delivery of substances given by routes other than the digestive tract, and covers administration routes, such as, intravenous, intra-arterial, intramuscular, intracerebroventricular, intraosseous intradermal, intrathecal, intraperitoneal administration, intravesical infusion and intracavernosal injection.

Typically “parenteral administration” refers to intravenous administration. A particular form of parenteral administration refers to the delivery by intravenous administration of nutrition (“parenteral nutrition”). Parenteral nutrition is called “total parenteral nutrition” when no food is given by other routes.

“Parenteral nutrition” is a isotonic or hypertonic aqueous solution (or solid compositions to be dissolved, or liquid concentrates to be diluted to obtain an isotonic or hypertonic solution) comprising a saccharide, such as, glucose and further comprising one or more of lipids, amino acids.

“Peripheral blood mononuclear cells” (PBMCs) constitute a very important part of our peripheral immune system. The PBMCs consist mainly of monocytes, T-cells and B-cells, and smaller amounts of NK cells and dendritic cells of both myeloid and plasmacytoid origin. Bone-resorbing osteoclasts are formed from hemopoietic cells of the monocyte-macrophage lineage and the development of osteoclasts is called osteoclastogenesis.

A “critically ill” patient is a patient receiving intensive-care where under mechanical ventilation and intensive-care nutrition support under the form of parenteral and/or enteral nutrition and is being treated in an Intensive Care Unit (“ICU”), e.g., of a hospital. Often the intensive-care nutrition support concerns parenteral nutrition) (PN), which is feeding an individual intravenously, bypassing the usual process of eating and digestion. The person, thereby, receives nutritional formulas that contain nutrients, such as, glucose, amino acids, lipids and added vitamins and dietary minerals. It is called total parenteral nutrition (TPN) or total nutrient admixture (TNA) when no food is given by other routes. This unnatural way of feeding the body is far from perfect and comes with several significant complications. Administration of large volumes of blood products, especially packed red cells, undergoing dialysis, especially continuous veno-venous hemofiltration, receiving multiple antibiotics, having a pulmonary artery catheter or an arterial blood pressure catheter inserted can be further part of the intensive care. These further criteria for critically ill patients are exemplary only, and one skilled in the art will understand that other indicia of a patient in a critically ill state are possible and are considered to be encompassed by the term “critically ill,” as it is used herein. The typical condition of treatment in an ICU environment and patient condition at entry leads to disease attributes [MeSH Descriptor C23.550.291] and pathological conditions [MeSH Descriptor C23] recognized by the medical community as a typical disorder of Critical Illness [MeSH Descriptor C23.550.291.625] a disease or state in which death is possible or imminent and with typical complication. Important complications specifically induced by the critical illness condition are critical illness myopathy (2012 ICD-9-CM Diagnosis Code 359.81) and critical illness polyneuropathy (2011 ICD-10-CM G62.81) and “Osteopenia” (ICD-10 M85.8, ICD-9 733.90, DiseasesDB 29870 or MeSH D001851), which is a condition where bone mineral density is lower than normal more specifically (osteopenia is defined as a bone mineral density T-score between −1.0 and −2.5). Another important indication related to critical illness condition of critically ill patients is critical illness bone Atrophy. Critically ill patients often present with extreme bone loss, are at increased risk of fractures, such as, stress fractures (ICD-10-CM 733.93 Stress fracture of tibia or fibula, 733.94 Stress fracture of the metatarsals, 733.95 Stress fracture of other bone, 733.96 Stress fracture of femoral neck, 733.97 Stress fracture of shaft of femur and/or 733.98 Stress fracture of pelvis) during rehabilitation, and can experience impaired healing of traumatic and surgical bone fractures. Metabolic Bone Disease Following ICU Admission, Critical Illness Related Metabolic Bone Disease, Osteoporosis Secondary to ICU Admission is a serious disorder that usually needs post-ICU ambulatory care. The bone resorption related to this disorder occurs in nearly all prolonged critically ill patients (B. Nierman (1998) Chest 114(4): 1122-8). It very often leads to complications, such as, hip fracture and vertebral fracture (A. Volk (2009) Am fam Physician 79(6): 459-64).

Critical illness uniformly predisposes to prolonged dependency on vital organ support. Lean tissue wasting of skeletal muscle and bone characterize the hypercatabolic state of prolonged critical illness (Hermans G, Vanhorebeek I, Derde S, Van den Berghe G. Metabolic aspects of critical illness polyneuromyopathy. Crit. Care Med. 2009; 37(10 Suppl):S391-7.). Specifically, circulating biomarkers of bone breakdown are extremely elevated whereas markers of bone formation are low (Van den Berghe G, Van Roosbroeck D, Vanhove P, Wouters P J, De Pourcq L, Bouillon R. Bone turnover in prolonged critical illness: effect of vitamin D. J Clin Endocrinol Metab. 2003; 88(10):4623-32.), an imbalance, which may predispose critically ill patients to skeletal morbidity, such as, impaired fracture healing, osteoporosis, and increased risk of new fractures during rehabilitation. Examples of the latter are patients who underwent sternotomy for cardiac surgery and who develop slow or poor healing of the sternal fracture. This is referred to as sternal dehiscence, a serious complication that carries high morbidity and even mortality (Hashemzadeh K, Hashemzadeh S. In-hospital outcomes of delayed sternal closure after open cardiac surgery. J Card Surg. 2009; 24(1):30-3.). Normal sternal bone healing may be further threatened by the necessity, in complicated cases, to leave the sternotomy wound open for a number of days in order to reduce intrathoracic pressure and allow the damaged heart to recover (Abid Q, Podila S R, Kendall S. Sternal dehiscence after cardiac surgery and ACE inhibitors [correction of ACE type 1 inhibition]. Eur J Cardiothorac Surg. 2001; 20(1):203-4.). In addition, a recent retrospective case-cohort study has revealed a significant increase in fracture risk in patients who were in intensive care for a variety of reasons (Orford N R, Saunders K, Merriman E, Henry M, Pasco J, Stow P, et al. Skeletal morbidity among survivors of critical illness. Crit. Care Med. 2011; 39(6):1295-300.). Together these data suggest a strong link between prolonged critical illness and skeletal morbidity.

Normal bone turnover depends on a tight coupling between function of mature osteoclasts, osteoblasts, and vascularization. This coupling requires a complex equilibrium of mechanical, endocrine, and nutritional factors. Prolonged critically ill patients are immobilized and suffer from a wide variety of endocrine and inflammatory disturbances, including hypercortisolism, hyposomatotropism, secondary hypothyroidism, hypogonadism, vitamin D deficiency and elevated cytokine levels, such as, tumor necrosis factor α (TNF-α), interleukin (IL-) 1 (IL-1) and IL-6 (2), all of which may contribute to the increase in bone resorption markers and reduced bone formation markers observed.

In other disease states characterized by excessive bone loss, such as, postmenopausal osteoporosis or cystic fibrosis, increased osteoclast formation and activity of peripheral blood mononuclear cells (PBMC) has been reported (D'Amelio P, Grimaldi A, Pescarmona G P, Tamone C, Roato I, Isaia G. Spontaneous osteoclast formation from peripheral blood mononuclear cells in postmenopausal osteoporosis. FASEB J. 2005; 19(3):410-2), both in the presence and absence of the canonical osteoclast activation factors, receptor activator of NF-κB ligand (RANKL) and macrophage colony stimulating factor (M-CSF) (Neale S D, Schulze E, Smith R, Athanasou N A. The influence of serum cytokines and growth factors on osteoclast formation in Paget's disease. QJM. 2002; 95(4):233-40). Increased osteoclast formation in a number of disease states characterized by excessive bone loss, such as, postmenopausal osteoporosis, rheumatoid arthritis and cystic fibrosis appear to be related to elevated serum TNF-α, IL-1 and IL-6 levels (Shead E F, Haworth C S, Barker H, Bilton D, Compston J E. Osteoclast function, bone turnover and inflammatory cytokines during infective exacerbations of cystic fibrosis. J Cyst Fibros. 2009; Kaur K, Hardy R, Ahasan M M, Eijken M, van Leeuwen J P, Filer A, Thomas A M, Raza K, Buckley C D, Stewart P M, Rabbitt E H, Hewison M, Cooper M S. Synergistic induction of local glucocorticoid generation by inflammatory cytokines and glucocorticoids: implications for inflammation associated bone loss. Ann Rheum Dis. 2010). This is in contrast with our findings, where inflammatory cytokines do not appear to contribute to increased osteoclast formation. Hence these data show that increased osteoclast formation during critical illness differs from classical bone resorption diseases, which is a very novel insight. New bone formation and fracture healing requires precursor cells, located in the periosteum, a specialized connective tissue fanning a thin but tough fibrous membrane firmly anchored to bone. Human periosteal-derived cells (hPDCs) have been utilized as a clinically relevant model to examine the effects of illnesses on bone formation.

Until now, no studies have directly investigated the effect of critical illness on bone metabolism and repair at the tissue and cellular level. We here performed a series of studies to address this issue. Although biomarkers suggested excessive bone loss during critical illness, this had not been confirmed at the tissue level. Hence, we first quantified bone loss in an in vivo rabbit model of prolonged critical illness. Second, in an in vitro model, we investigated whether PBMCs isolated from critically ill patients are predisposed, more than those from healthy subjects, to differentiate into osteoclasts and assessed their osteoclastic activity. In addition, we studied the role of humoral factors in the patient's serum on these processes. Third, we assessed osteogenesis during critical illness, in an in vitro model using human periosteal cells, and in an in vivo murine model.

### EXPERIMENTAL

Herein it was demonstrated that osteoclasts isolated from ICU patient PBMCs form sooner than osteoclasts from healthy controls, whereby that osteoclasts isolated from patient PBMCs form in the absence and presence of RANKL and M-CSF.

Furthermore, Autophagy marker p62 protein expression in healthy and sick (from ICU patient) osteoclasts (RANKL/MCSF stimulated) studies revealed that p62 protein is increased in osteoclasts from critically ill patients, suggesting a deficiency in autophagy, demonstrating role of autophagy increased osteoclast formation and activity in critical illness.

A dose finding demonstrated that spermidine inhibits osteoclast resorption. While osteoclast formation is significantly increased in Stimulated (+RANKL/MCSF) PBMCs from sick (ICU) patients vs. healthy controls, this increase is completely blocked by spermidine treatment.

It has been also demonstrated that osteoclasts isolated from patient PBMCs form sooner than osteoclasts from controls. Osteoclasts isolated from patient PBMCs form in the absence and presence of RANKL and M-CSF. p62 expression is increased in patient osteoclasts, which suggests deficient autophagy. Increased osteoclast formation and activity in patient PBMC's is completely blocked with spermidine.

The disclosure relates to novel therapeutic, methods useful for the treatment or prevention of such bone deterioration or bone loss in the group of severe, life threatening diseases. Moreover, the invention provides compositions and treatments of Critical Illness Bone Atrophy, Metabolic Bone Disease Following ICU Admission, Critical Illness Related Metabolic Bone Disease or Osteoporosis Secondary to ICU Admission.

The disclosure relates to novel therapeutic, methods useful for the treatment or prevention of stress fractures (ICD-10-CM 733.93 Stress fracture of tibia or fibula, 733.94 Stress fracture of the metatarsals, 733.95 Stress fracture of other bone, 733.96 Stress fracture of femoral neck, 733.97 Stress fracture of shaft of femur and/or 733.98 Stress fracture of pelvis) of patients that experienced extreme bone loss due to Metabolic Bone Disease Following ICU or due to bone resorption during ICU treatment that occurs in nearly all prolonged critically ill patients.

The methods, treatments and compositions of the invention, are also for in use to treat or prevent disuse osteoporosis also called bone disuse atrophy, disuse atrophy of bone (ICD-9-CM Diagnosis Code 733.03), a form of osteoporosis due to immobilization or inactivity.

In general, the disclosure can be considered as relating to the field of intensive care medicine. More specifically, it is based on the surprising finding that during critical illness there occurs an uncoupling between bone formation and degradation. In a rabbit model of critical illness, pQCT analysis of proximal tibiae revealed a decrease in bone mineral content.

This was investigated further in a clinically relevant human in vitro model of critical illness, where peripheral blood mononuclear cells (PBMCs) from critically ill patients formed mature, multi-nuclear actively resorbing osteoclasts both in the presence and absence of osteoclastogenic factors RANKL and M-CSF, potentially due to an increase in circulating osteoclast precursors detected by flow cytometry. Treatment with 10% critically ill patient serum further increased osteoclast formation and activity.

No apparent differences in osteogenesis were observed in human perisoteal-derived cells (hPDCs) treated with patient serum in vitro; however, a decrease in the expression of VEGF-R1 suggested impaired vascularization. This was confirmed using serum-treated hPDCs implanted onto calcium phosphate scaffolds in a murine in vivo model of bone formation, where decreased vascularization and increased osteoclast activity led to a decrease in bone formation in scaffolds with patient serum-treated hPDCs. In summary, we have shown that the disruption in bone metabolism observed in critically ill patients is predominantly explained by an increase in osteoclastogenesis coupled with a moderate decrease in bone formation, which may be due to deficient vascularization.

Provided are compositions and pharmaceutical compositions for suppressing osteoclastogenesis. The disclosure is based on the surprising finding of a novel mechanism that is at the basis of enhanced osteoclastogenesis in critically ill patients, which is a cause of Critical Illness Bone Atrophy, Metabolic Bone Disease Following ICU Admission, Critical Illness Related Metabolic Bone Disease or Osteoporosis Secondary to ICU Admission and is the reason why critically ill patients often present with extreme bone loss and are at increased risk of fractures, such as, stress fractures (ICD-10-CM 733.93 Stress fracture of tibia or fibula, 733.94 Stress fracture of the metatarsals, 733.95 Stress fracture of other bone, 733.96 Stress fracture of femoral neck, 733.97 Stress fracture of shaft of femur and/or 733.98 Stress fracture of pelvis) during rehabilitation and can experience impaired healing of traumatic and surgical bone fractures, can be determined by measuring peptides indicating a high physiological release of the potent vasoconstrictors as endothelin-1, in critically ill patients with high levels of adrenomedullin can exert a beneficial, potentially life-saving effect. Based on the novel mechanism of suppressing osteoclastogenesis compounds have been demonstrated to suppress such enhanced osteoclastogenesis and compositions and pharmaceutical compositions have been presented to treat or prevent Critical Illness Bone Atrophy, Metabolic Bone Disease Following ICU Admission, Critical Illness Related Metabolic Bone Disease or Osteoporosis Secondary to ICU Admission.

We have determined that the treatment of patient's peripheral blood mononuclear cell (PBMCs), in particular, PBMCs of critically ill patients with autophagy inducers can be used to suppress enhanced osteoclastogenesis. It turned out that a particular advantage is that such autophagy inducers can be targeted to particularly treat the PBMCs. We have also determined that for such treatment, surprisingly low doses of autophagy inducers can be used, possibly because patient's PBMCs are the treatment target cells. For instance, we used PBMCs from critically ill patients cultured for 14 days in critically ill patient serum and compounds added from d1-d14 and cells staining with TRAP for osteoclast formation at day 14 and demonstrated the inhibiting effect on critical illness enhanced osteoclast formation of low doses autophagy inducers, such as, Spermidine, Rapamycin, Everolimus, Promethazine and of methylation inhibitor, such as, the global methylation inhibitor-5-azacytidine.

### Example 1

### In Vivo Model of Critical Illness

Animals

All animals were treated according to the Principles of Laboratory Animal Care formulated by the U.S. National Society for Medical Research and the Guide for the Care and Use of Laboratory Animals prepared by the National Institutes of Health. The study protocol was approved by the Leuven University ethical review board for animal research (P 108/2009). The model has been described in detail previously (Weekers F, Giulietti A P, Michalaki M, Coopmans W, Van Herck E, Mathieu C, et al. Metabolic, endocrine, and immune effects of stress hyperglycemia in a rabbit model of prolonged critical illness. Endocrinology. 2003; 144 (12):5329-38.). At day-1, adult, 3- to 4-month-old male New Zealand white rabbits weighing 3 kg were anesthetized and catheters were inserted in the right jugular vein and right carotid artery, allowing intravenous infusion of insulin and fluids and repetitive blood sampling. Fluid resuscitation consisting of hartmann (Baxter, Lessines, Belgium), enriched with glucose to prevent hypoglycemia, was started after the operation. At day 0, animals were anesthetized again, and a full thickness 15-20% body surface area third-degree burn injury was inflicted on the flanks, after having performed a paravertebral block with lidocaine (Xylocalne, AstraZeneca, Brussels, Belgium). Fluid resuscitation consisted of hartmann-glucose, and was changed to total parenteral nutrition at day 1. Animals were targeted to hyperglycemia, with concomitant hyperinsulinemia. This model has been validated as representative of the human critically ill condition (9, 36). Daily arterial blood was sampled and ionized calcium levels were measured immediately by a blood gas analyzer (ABL725, Radiometer, Copenhagen, Denmark). A daily 4 cc blood sample was collected and plasma stored at −80° C. until further analysis. On day 7 animals were sacrificed and the left and right tibiae were dissected, dipped in polyvinylalcohol (PVA) and snap-frozen in liquid nitrogen, after which they were stored at −80° C. until further analysis. For comparison, healthy rabbits were sacrificed and tissue samples were collected, as described above. Plasma osteocalcin was measured by an in-house rabbit osteocalcin RIA, as previously described, (Van den Berghe G, Van Roosbroeck D, Vanhove P, Wouters P J, De Pourcq L, Bouillon R. Bone turnover in prolonged critical illness: effect of vitamin D. J Clin Endocrinol Metab. 2003; 88(10):4623-32, Bouillon R, Vanderschueren D, Van Herck E, Nielsen H K, Bex M, Heyns W, et al. Homologous radioimmunoassay of human osteocalcin. Clin Chem. 1992; 38(10):2055-60.).

Peripheral Quantitative Computed Tomography

Trabecular and cortical BMC and BMD and the geometry of the right tibia were assessed ex vivo by peripheral quantitative computed tomography (pQCT) using the Stratec XCT Research densitometer (Norland Medical Systems, Fort Atkinson, Wis., USA). Slices of 0.2 mm thickness were obtained using a voxel size of 0.070 mm. One scan was taken 2.4 mm from the proximal end of the tibia to measure trabecular volumetric density. The trabecular bone region was defined by setting an inner threshold to 30% of the total cross-sectional area. A second scan was taken 7 mm from the proximal end of the tibia (an area containing mostly cortical bone). These mid-diaphyseal scans were performed to determine cortical volumetric density, cortical thickness, and periosteal and endocortical perimeter. Cross-sectional moment of inertia, based on cortical bone measurements, was calculated using the circular ring model algorithm of the software program.

### Example 2

### In Vitro Model of Bone Resorption During Critical Illness

Experimental Subjects

Human peripheral blood was collected from prolonged critically ill patients (n=12, 26-80 years of age, mean age 57±16.39 years of age) and healthy control volunteers, matched for age, sex and body mass index (BMI) (n=12, 23-81 years of age, mean age 57±17.44 years of age). All protocols were approved by the Institutional Review Board of the Leuven University. Written informed consent was obtained from all healthy volunteers and from the patients or, when the patient was unable to give consent, from the closest family member. Prior to sample collection, it was ensured that no steroidal drugs or bisphosphonates had been taken by patients or healthy volunteers in the past 12 months.

Flow Cytometry

Osteoclast precursors were detected in healthy volunteers or critically ill patients by staining fresh blood samples with allophycocyanin (APC)-conjugated anti-VNR, phycoerythrin (PE)-conjugated anti-CD14 and fluorescein isothiocyanate (FITC)-conjugated anti-CD11b, or with the corresponding isotype control followed by incubation at 4° C. for 30 min. A human Fc-gamma receptor (FcγR)-binding inhibitor was used to inhibit non-specific FcγR mediated binding. We treated double positive CD14+/CD11b+ cells as early osteoclast precursors, and triple positive CD 14+/CD11b+/VNR+ cells as osteoclast precursors, according to previous literature (22, 38). Flow cytometry was performed on a FACSCALIBUR® flow cytometer (BD Biosciences, Erembodegem, Belgium.). The expression of membrane antigens was analyzed using BD FACSDIVA® software (BD Biosciences).

Peripheral Blood Mononuclear Cell Isolation and Culture

PBMCs were isolated from the whole blood of patients or healthy volunteers by means of Ficoll-Paque Plus (GE Healthcare, Brussels, Belgium) density gradient centrifugation according to the manufacturer's instructions. Heparinized whole blood (20 ml) was drawn from each individual and diluted 1:1 with alpha minimal essential medium (α-MEM; Lonza, Braine-l'Alleud, Belgium), before being layered over Ficoll-Paque and centrifuged at 450 g for 40 min. The mononuclear cell interface was then removed and washed 3 times with α-MEM. Cells were stored in liquid nitrogen until use and seeded at a density of 5×10⁵ cells per well. All cultures were performed in quadruplicate in 16-well culture slides (VWR, Leuven, Belgium) or 16-well BD Biocoat Osteologic Slides (BD Biosciences), and cells were cultured in α-MEM supplemented with 10% FBS, 100 IU/ml penicillin and 100 μg/ml streptomycin (“complete medium”), or complete medium plus M-CSF (25 ng/ml) and RANKL (30 ng/ml). In order to study the effect of circulating factors on osteoclast formation, PBMCs were also grown in α-MEM containing 10% human healthy volunteer serum (HS) or 10% patient serum (PS), in the presence or absence of M-CSF (25 ng/ml) and RANKL (30 ng/ml). For cytokine neutralization experiments, PBMCs were grown in α-MEM/10% PS for 14 days, in the presence of varying concentrations of anti-TNF-α and/or anti-IL-6 (R&D Systems, Abingdon, UK). All cultures were maintained at 37° C. in a humidified atmosphere with 5% CO2.

Osteoclast Formation and Activity

After 14 days, PBMCs were fixed and stained for the osteoclast-specific marker tartrate-resistant acid phosphate (TRAP), and the nuclear marker DAPI, as previously described. The formation of TRAP positive multi-nucleated (more than three nuclei) cells was quantified by counting the multi-nuclear stained cells in each well (D'Amelio P, Grimaldi A, Pescarmona G P, Tamone C, Roato I, Isaia G. Spontaneous osteoclast formation from peripheral blood mononuclear cells in postmenopausal osteoporosis. FASEB J. 2005; 19(3):410-2.). Filamentous (F)-actin rings representing active osteoclasts were visualized after staining of the actin cytoskeleton with the toxin phalloidin conjugated to FITC, and quantified by counting, as described above. To evaluate osteoclast activity, cells were removed from the hydroxyapatite-coated wells with 14% sodium hypochlorite, and the mineral layer was stained with Von-Kossa, as previously described, (Ariyoshi W, Takahashi T, Kanno T, Ichimiya H, Shinmyouzu K, Takano H, et al. Heparin inhibits osteoclastic differentiation and function. J Cell Biochem. 2008; 103(6):1707-17). Lacunar resorption was determined by measuring the total area unstained by Von-Kossa (total area resorbed) using ImageJ software (National Institute of Health, MD, USA), and was expressed as the total percentage of the surface reabsorbed.

### Example 3

### In Vitro Model of Osteoblast Differentiation During Critical Illness

Cell Culture

Human Periosteal Derived Cells (hPDCs) were obtained from the Laboratory for Skeletal Development and Joint Disorders, Katholieke Universiteit Leuven, Leuven, Belgium. Cells were expanded in monolayer at 37° C. in a humidified atmosphere of 5% CO2 in growth medium, which consisted of high-glucose Dulbecco's modified Eagle medium (DMEM; Invitrogen, Merelbeke, Belgium) containing 10% γ-irradiated and filtered FBS (Gibco), 1% sodium pyruvate (100 mM; Invitrogen) and 1% antibiotic-antimycotic solution (100 units/ml penicillin, 100 μg/ml streptomycin and 0.25 μg/ml amphotericin B; Invitrogen). The medium was replaced every 3 days. All experiments were carried out with expanded cell populations between passage 5 and 7, with a seeding density of 4500 cells/cm². After 48 hours in culture, the growth medium of in vitro osteogenic assays was replaced using osteogenic medium, which consisted of FBS-free growth medium supplemented with 100 nM dexamethasone (Sigma-Aldrich, Bornem, Belgium), 10 mM glycerol-2-phosphate disodium salt hydrate (Sigma-Aldrich), 50 μM L-ascorbic acid 2-phosphate sesquimagnesium salt hydrate (Sigma-Aldrich) and 10% serum (10% FBS, 10% PS, or 10% HS). The medium was replaced every 3 days.

In Vitro Mineralization Assay

hPDCs were analyzed for in vitro mineralization using Alizarin Red to detect mineral deposits, as previously described. Briefly, hPDCs were seeded at 4500 cells/cm2 and treated for 21 days with osteogenic medium and FBS, HS or PS. At day 21, the cultures were rinsed with PBS, fixed with 4% formaldehyde, and then rinsed with distilled water before staining with an Alizarin Red solution (Sigma; pH 4.2). Nonspecific staining was rinsed off carefully with distilled water. Quantification of calcium mineral deposits was performed by dissolving the dye with 10% cetylpyridinium chloride (in demineralized water) for 10 min at RT. Absorbance was measured spectrophotometrically at 570 nm.

### Gene Expression Analysis

hPDCs were seeded at 4500 cells/cm2 and treated for 7 days with osteogenic medium and FBS, HS or PS. Total RNA was isolated using the RNeasy kit (Qiagen Benelux, Venlo, Netherlands) and cDNA was synthesized with the SuperScript III First Strand synthesis system for real-time PCR (Invitrogen). Quantitative real-time SYBR Green (Invitrogen) PCR was performed according to the manufacturer's protocol, with mRNA levels normalized to β-actin expression. SYBR Green qPCR primers were designed to span an intron so that only RNA-specific amplification was possible (RUNX2-F, 5′-CGCATTCCTCATCCCAGTAT-3′ (SEQ ID NO:1); RUNX2-R, 5′-GCCTGGGGTCTGTAATCTGA-3′ (SEQ ID NO:2); COL1A1-F, 5′-GACGAAGACATCCCACCAAT-3′ (SEQ ID NO:3); COL1A1-R, 5′-AGATCACGTCATCGCACAAC-3′ (SEQ ID NO:4); ALP-F, 5′-GGACATGCAGTACGTAGCTGA-3′ (SEQ ID NO:5); ALP-R, 5′-GTCAATTCTGCCTCCTTCCA-3′ (SEQ ID NO:6); VEGFA-F, 5′-CCCACTGAGGAGTCCAACAT-3′ (SEQ ID NO:7); VEGFA-R, 5′-GCATTCACATTTGTTGTGCTG-3′ (SEQ ID NO:8); VEGF-R1-F, 5′-AAGCAAACCACACTGGCTTC-3′ (SEQ ID NO:9); VEGF-R1-R, 5′-CGGGGATTTCACTGTACATCT-3′ (SEQ ID NO:10)). Total RNA samples subjected to cDNA synthesis reactions in the absence of reverse transcriptase were included as negative controls and relative differences in expression were calculated using the 2ΔCT method (Livak K J, Schmittgen T D. Analysis of relative gene expression data using real-time quantitative PCR and the 2(-Delta Delta C(T)) Method. Methods. 2001; 25(4):402-8).

Western Immunoblotting

For protein expression analysis, hPDCs were seeded at 4500 cells/cm2 and treated for 7 days with osteogenic medium and FBS, HS or PS. After 7 days, the cell monolayer was washed with ice-cold 1×PBS, and lysates were prepared by adding lysis buffer (RIPA buffer; 50 mM Tris, 150 mM NaCl, 0.1% SDS, 0.5% Na-deoxycholate, 1% TritonX-100, 1 mM phenylmethanesulfonylfluoride (PMSF)) containing protease and phosphatase inhibitors. Lysates were scraped into a 1.5 ml microcentrifuge tube, and centrifuged for 15 min at 4° C. Supernatants were transferred to fresh microcentrifuge tubes and stored at −80° C. The protein concentration was determined by the Pierce BCA Protein Assay (VWR). Equal amounts of protein were loaded onto each lane of a 4-12% Bis-Tris gel and subjected to electrophoresis under reducing conditions. After blotting, polyvinylidene difluoride membranes were blocked for one hour (5% milk powder in 0.1% PBS/Tween) and incubated with Rabbit anti-human VEGFR1 primary antibody (Abeam, Cambridge, UK; 1:500) overnight at 4° C. Binding of Goat anti-rabbit IgG HRP secondary antibody (DakoCytomation, Glostrup, Denmark; 1:2000) was visualized by enhanced chemiluminescence (ECL). Normalization for total protein was performed by re-probing the membrane with Mouse anti-mouse Beta-actin (Abeam, Cambridge, UK; 1:1000) for 1 hour at RT, followed by Goat anti-mouse IgG HRP (DakoCytomation; 1:2000) for 1 hour at RT.

### Example 4

### In Vivo Model of Bone Formation During Critical Illness

In Vivo Bone Formation

In vivo bone formation was analyzed as described recently (Roberts S J, Geris L, Kerckhofs G, Desmet E, Schrooten J, Luyten F P. The combined bone forming capacity of human periosteal derived cells and calcium phosphates. Biomaterials. 32(19):4393-405). Briefly, hPDCs (passage 5) were seeded at 4500 cells/cm2 and treated for 7 days with osteogenic medium and FBS, HS or PS. After 7 days, the cells were trypsin released, centrifuged and resuspended at a concentration of 50 million cells/ml. Subsequently, 20 μl of the cell suspension was applied to the upper surface of each scaffold. To allow cell attachment, the seeded scaffolds were incubated overnight at 37° C. After incubation, the constructs were either directly implanted subcutaneously in the back at the cervical region of NMRI-nu/nu mice. The remaining cells in the supernatant were counted to estimate the seeding efficiency, which was calculated as follows: [(number of seeded cells−number of cells in the supernatant)/number of seeded cells]*100. The implants were collected after 56 days of implantation. Each explant was fixed in 4% formaldehyde, scanned by μCT, decalcified in EDTA/PBS (pH 7.5) for 2 weeks, paraffin embedded and processed for histology. All procedures on animal experiments were approved by the local ethical committee for Animal Research (Katholieke Universiteit Leuven). The animals were housed according to the guidelines of the Animalium Leuven (Katholieke Universiteit Leuven).

Bone Quantification

To quantify ectopic bone formation, μCT was used to quantify the volume of new bone faulted in three dimensions by segmenting the newly formed mineralized tissues from the calcium phosphate grains in each material. For segmentation, a manually selected, but consistent global threshold value was used for each scaffold. The choice of threshold value was confirmed by visual comparison to the corresponding histological sections.

Histological Analysis

Histological staining was performed on paraffin embedded sections of cell/biomaterial constructs. Briefly, paraffin sections were deparaffinized in HISTOCLEAR™ (Laborimpex, Brussels, Belgium) and methanol, and rinsed with distilled water. For TRAP staining the tissue sections were incubated in TRAP buffer (50 mm Sodium Acetate, 30 mm Sodium Tartrate, 0.1% Triton X-100, pH 5) for 20 min. This was then replaced with TRAP stain (0.5 mg/ml napthol AS-MX phosphate and 1.1 mg/ml Fast red violet LB in TRAP buffer prewarmed to 37° C.) for 1 h before washing twice in PBS, counterstaining with hematoxylin and mounting with aqueous mounting medium. TRAP-positive osteoclasts were visualized using fluorescent microscopy, and quantified using IMAGEJ® software (National Institutes of Health, USA). For CD31 immunohistochemistry, the tissue sections underwent antigen retrieval in proteinase K for 15 min at 95° C. and were blocked in 1% normal rabbit serum, before being incubated overnight at 4° C. with Rat Anti-mouse CD31 (BD Pharmingen; 1:20). Sections were then probed with the biotinylated rabbit anti-rat IgG antibody (Vector Laboratories; 1:100), and the signal amplified with the Vector Laboratories “ABC system.” The signal was detected using 3,3′-Diaminobenzidine (DAB) the sections counterstained with hematoxylin, and the number of CD31 positive vessels per scaffold was visually quantified.

Statistical Analysis

The data were processed using the Statistical software package StatView 5.0.1 (SAS Institute Inc.). Student's t test was used for the comparison of normally distributed data (presented as mean±SD), and the Mann-Whitney U test for data that were not normally distributed (presented as median and IQR, unless otherwise indicated). P values less than or equal to 0.05 were considered statistically significant. Statistical significance is indicated on all graphs as follows: *: p<0.05, **: p<0.01, ***: p<0.001.

### Example 5

### Osteoclasts in Critical Illness

Isolation of osteoclasts from human peripheral blood mononuclear cells.

15-20 ml peripheral blood collected in lithium-heparin tubes.

Mixed with αMEM and add to Ficoll-Paque, centrifuged and mononuclear cells separated.

Plate down cells in αMEM with 10% serum and +/−RANKL and MCSF.

By week 3 extensive resorption can be observed and osteoclast formation can be measured by staining for TRAP. Also Markers of osteoclast activity significantly increased during critical illness.

### Example 6

Spermidine-induced reduction of osteoclast activity.

On peripheral blood mononuclear cells (PBMCs) isolated from ICU patients (4) and healthy controls (5) invention demonstrates by a dose response experiment using healthy cells only (FIG. 1) that osteoclast activity is reduced at 10⁻⁶, 10⁻⁷, and 10⁻⁸M Spermidine (Sigma S4139, lot 0001411140).

### Example 7

Spermidine-induced reduction of osteoclast activity in critically ill derived PBMCs (stimulated).

In the next experiment, we measured osteoclast formation (FIG. 2), in healthy and sick (from critically ill) stimulated (with RANKL and MCSF) PBMCs with and without spermidine (10⁻⁸ M). By day 7 there was already a noticeable difference between the treatment groups (FIG. 2A top panel), and when the cells were stopped at day 14, there was a significant increase in TRAP positive multinuclear cells (indicative of increased osteoclast formation) in sick vs. healthy cells, and a significant reduction in TRAP positive cells (indicative of reduced osteoclast activity) in spermidine treated sick PBMCs compared to “control” sick PBMCs (FIG. 2A, bottom 2 panels, and FIG. 2B). Spermidine treatment did not, however, have a significant effect on the healthy PBMCs.

### Example 8

Spermidine-induced reduction of osteoclast activity in critically ill derived PBMCs (unstimulated).

We also looked at spontaneous osteoclast formation in healthy and sick unstimulated (no RANKL or MCSF) PBMCs, with and without spermidine. As previously, we found a significant increase in osteoclast formation in sick PBMCs vs. healthy, and this increase was completely knocked down with spermidine (FIGS. 3A and B). Again we found there was no effect on the healthy cells with spermidine.

### Example 9

Osteoclast resorption measurement in stimulated PBMCs, unstimulated PBMCs of healthy and sick (critically ill) subjects and the effect of spermidine thereon.

In addition to osteoclast formation, we also looked at osteoclast activity, by measuring osteoclast resorption of a hydroxyapatite layer. The area of hydroxyapatite “eaten into” can be measured using ImageJ software. In stimulated PBMCs, there was a significant increase in resorption in sick cells compared to healthy, and again this increased was reduced with spermidine treatment (FIGS. 4A and B,). In unstimulated PBMCs, only a very small amount of resorption was seen in any of the treatment groups (FIGS. 5A and B), however, there was a small increase in resorption in the sick cells compared to healthy.

### Example 10

Effects of spermidine on autophagy markers.

We also measured markers of autophagy in the cell lysates from these experiments. The concentration of proteins we obtained were quite low, and there was insufficient protein to measure anything by western blotting in the “sick spermidine” group. However, we were able to measure p62 in the healthy, sick, and healthy spermidine cells, and found that p62 protein was significantly increased in sick cells compared to healthy, suggesting an accumulation of p62 and, therefore, defective autophagy in sick osteoclasts. There was no effect of spermidine on the healthy PBMCs (FIG. 6).

### Example 11

Trabecular, but not cortical, bone is lost in a rabbit model of critical illness.

A 7-day rabbit model of critical illness has previously been validated to examine localized effects of critical illness (Weekers F, Giulietti A P, Michalaki M, Coopmans W, Van Herck E, Mathieu C, et al. Metabolic, endocrine, and immune effects of stress hyperglycemia in a rabbit model of prolonged critical illness. Endocrinology. 2003; 144(12):5329-38). Like in human critically ill patients, serum ionized calcium levels were significantly reduced in critically ill rabbits (FIG. 19A). Infliction of the critical illness evoked an immediate decrease in ionized calcium on day 1 (3.4%), which remained lower than in healthy rabbits for the entire 7-day period of illness. Also similar to what is described in human patients, serum osteocalcin levels were lowered in critically ill rabbits by day 4 of illness (FIG. 19B). In order to examine the direct effect of critical illness on skeletal integrity, bone mineral content (BMC) and bone mineral density (BMD) were measured in the proximal tibia after 7 days of illness. Importantly, trabecular BMC and BMD were significantly lower in critically ill rabbits than in healthy controls (FIGS. 19C and D), but no differences were observed in cortical BMC, BMD or thickness (FIGS. 19E and F).

### Example 12

Increased osteoclast formation and activity in critically ill patients occurs spontaneously and is potentiated with autologous patient serum.

To further examine the cause of bone loss during critical illness in a clinically relevant model, PBMCs were isolated and pooled from an age- sex- and BMI-matched set of critically ill patients and healthy controls, and analyzed for osteoclast formation and activity.

To establish whether cells from critically ill patients are programmed to differentiate into osteoclasts before any in vitro manipulation, the number of osteoclast precursors present in critically ill patient or healthy control peripheral blood was assessed by Fluorescence Activated Cell Sorting (FACS) analysis. Double positive CD14+/CD11b+ cells are considered to be early osteoclast precursors, and triple positive CD14+/CD11b+/VNR+ cells are considered to be more mature circulating osteoclasts (D'Amelio P, Grimaldi A, Pescarmona G P, Tamone C, Roato I, Isaia G. Spontaneous osteoclast formation from peripheral blood mononuclear cells in postmenopausal osteoporosis. FASEB J. 2005; 19(3):410-2, Roato I, Grano M, Brunetti G, Colucci S, Mussa A, Bertetto O, et al. Mechanisms of spontaneous osteoclastogenesis in cancer with bone involvement. FASEB J. 2005; 19(2):228-30). There were more early osteoclast precursors (CD14+, CD11b+) in blood from critically ill patients than in blood from healthy controls (FIGS. 20A and 20B) with only a trend for more mature circulating osteoclasts (CD14+, CD11b+, VNR+).

To confirm that these precursor cells indeed form active osteoclasts, the isolated PBMCs were cultured for 14 days, with and without RANKL and M-CSF, and the number and activity of osteoclasts in patients were compared with healthy matched controls. After 14 days of culture with RANKL and M-CSF, the formation of mature, multi-nuclear (≧3 nuclei, tartrate-resistant acid phosphatase (TRAP) positive) osteoclasts was 16.8-fold higher in cultures from critically ill patients than those from healthy controls (FIGS. 21A and B). Analysis of the activity of the osteoclasts was done by quantifying the degree of hydroxyapatite-resorption after culturing the cells for 21 days in hydroxyapatite-coated wells. This revealed a 36.2-fold higher resorption in patient PBMC cultures with RANKL and M-CSF as compared with controls (FIGS. 21A and C). PBMCs from critically ill patients also displayed spontaneous differentiation into osteoclasts (without RANKL and M-CSF; 23.8-fold increase vs. healthy cells), but the degree of hydroxyapatite resorption in spontaneously-formed osteoclast cultures from patients and controls was similar (FIGS. 21A and C).

To assess the impact of humoral factors in the patient's blood on the differentiation potential of PBMCs into osteoclasts, PBMCs from patients and controls were cultured in the presence of 10% autologous patient serum (PS) or 10% autologous healthy serum (HS). In the presence of RANKL and M-CSF, patient PBMCs cultured with 10% HS did not form significantly more mature, multi-nuclear TRAP positive osteoclasts per well than cells from healthy control grown under the same conditions (FIGS. 22A and B). In addition, also without RANKL and M-CSF, in the presence of 10% HS, minimal spontaneous osteoclast differentiation was observed, similar in patients and controls. However, in the presence of RANKL and M-CSF, the addition of 10% PS resulted in a 3.7-fold increase in osteoclast formation in patient PBMCs compared to cells grown with 10% HS. A similar increase was observed in “unstimulated” patient PBMC cultures (without RANKL and M-CSF), as when cultured in 10% PS, spontaneous osteoclast formation was increased by 6.1-fold compared to spontaneous osteoclast formation in patient PBMC cultures with 10% HS (FIGS. 22A and B). However, the addition of 10% PS to cultures of healthy control PBMCs did not increase osteoclast formation in the presence or absence of RANKL and M-CSF. These data suggest a unique interaction between patient cells and PS that is not present in healthy control cultures and PS.

The formation of F-actin rings displayed a similar pattern to that observed with the formation of TRAP positive multi-nuclear cells. A significant increase in F-actin ring formation was detected both with and without RANKL and M-CSF in patient cells cultured with 10% HS (30-fold and 6.25-fold, respectively) (FIGS. 22A and C), compared to healthy control cells cultured with 10% HS. A further 5.9-fold increase in F-actin ring formation was observed in patient cells grown in 10% PS, compared to 10% HS, and when grown without RANKL and M-CSF, an increase of 4.72-fold was still observed.

Resorption of hydroxyapatite was also significantly increased when 10% PS was added to patient PBMC cultures, but not when added to healthy control cultures (FIGS. 22A and D). With PS added to patients cells cultured in the presence of RANKL and M-CSF, 62.7% of the total surface area was resorbed as compared with 15.8% with HS (3.9-fold increase). A 1.63 fold increase in resorption was maintained in the absence of RANKL and M-CSF.

In order to assess whether the humoral factors that seem to interact with the primed PBMCs from critically ill patients could be the elevated levels of the major pro-inflammatory cytokines (Van den Berghe G, Van Roosbroeck D, Vanhove P, Wouters P J, De Pourcq L, Bouillon R. Bone turnover in prolonged critical illness: effect of vitamin D. J Clin Endocrinol Metab. 2003; 88(10):4623-32), cells were grown in 10% PS with or without neutralizing antibodies for IL-6 or TNF-α. Unexpectedly, however, adding anti-IL6 or anti-TNF-α further increased the formation of multi-nuclear TRAP positive osteoclasts in a dose-dependent manner. Strikingly, adding both antibodies in combination resulted in the formation of giant multi-nuclear osteoclasts (FIGS. 23A, B and C).

### Example 13

Critically ill patient serum does not affect osteoblast differentiation but reduces expression of angiogenesis markers in vitro.

In order to assess the impact of circulating factors during critical illness on bone formation, an in vitro model of osteogenesis during critical illness was set up. For this purpose, we used hPDCs, a pool of mesenchymal cells isolated from the human periosteum that have shown to proliferate, migrate and differentiate into chondrogenic and osteogenic lineages upon stimuli, such as, trauma, fracture or infection (De Bari C, Dell'Accio F, Vanlauwe J, Eyckmans J, Khan I M, Archer C W, et al. Mesenchymal multipotency of adult human periosteal cells demonstrated by single-cell lineage analysis. Arthritis Rheum. 2006; 54(4):1209-21, Roberts S J, Chen Y, Moesen M, Schrooten J, Luyten F P. Enhancement of osteogenic gene expression for the differentiation of human periosteal derived cells. Stem Cell Res. 7(2):137-44.). Cells were cultured for 21 days in osteogenic medium, as previously described, (Roberts S J, Chen Y, Moesen M, Schrooten J, Luyten F P. Enhancement of osteogenic gene expression for the differentiation of human periosteal derived cells. Stem Cell Res. 7(2):137-44, De Bari C, Dell'Accio F, Luyten F P. Human periosteum-derived cells maintain phenotypic stability and chondrogenic potential throughout expansion regardless of donor age. Arthritis Rheum. 2001; 44(1):85-95). As a late marker of hPDC osteoblast differentiation, calcium deposition was measured quantitatively using Alizarin Red staining on differentiated hPDCs.

Addition of fetal bovine serum (FBS), HS or PS to the osteogenic medium did not affect differentiation or mineralization of the cells (FIGS. 24A and B). Also gene expression analysis of osteogenic markers RUNX2 and COL1A1 (type I collagen) revealed no difference between PS and HS (despite a reduction in expression of these genes in both these groups as compared with cells grown in FBS) (FIGS. 24C and D). Expression of ALP (bone specific alkaline phosphatase) was also not different between PS and HS (FIG. 24E). These data suggest that osteogenic differentiation is unaffected by circulating factors during critical illness, assessed in vitro. Interestingly, expression of the angiogenesis factor vascular endothelial growth factor receptor (VEGF) alpha (VEGFA) was not significantly different between PS and HS groups (FIG. 24F), although a significant reduction in VEGF-receptor 1 (VEGFR1) expression with PS as compared with HS was detected at the gene and protein level, suggesting that angiogenesis may be compromised during bone formation and skeletal healing in critical illness (FIGS. 24G and H).

### Example 14

In an in vivo murine model of skeletogenesis, bone formation by critically ill patient cells is reduced, coinciding with increased osteoclast activity and reduced angiogenesis.

Bone formation is a multi-factorial process involving a tightly-regulated cascade of events which include the recruitment, proliferation, and differentiation of osteoprogenitor cells, along with the formation of a well-defined vascular compartment. Therefore, although no differences in osteogenesis in the presence of PS were observed in hPDCs in vitro, it was plausible that aberrant bone formation might still occur in vivo through deficient angiogenesis. In order to test this hypothesis, hPDCs were incubated with FBS, HS or PS for 7 days in basal medium, before being seeded onto calcium phosphate (CaP) NuOss□ scaffolds overnight, transplanted into NMRI-nu/nu mice, and incubated for 8 weeks (Roberts S J, Geris L, Kerckhofs G, Desmet E, Schrooten J, Luyten F P. The combined bone forming capacity of human periosteal derived cells and calcium phosphates. Biomaterials. 32(19):4393-405). Upon explanation and analysis of bone formation by μCT, scaffolds containing PS-treated hPDCs revealed significantly less mature bone than those containing HS-treated cells (FIG. 25A). No significant differences in calcium phosphate granules or fibrous tissue were observed. In view of the increased osteoclast activity observed in previous experiments, possibly contributing to bone hyperresorption in critical illness, the trend for an increase in osteoclastic activity in PS scaffolds, as detected by TRAP staining, was unsurprising (FIG. 25B). The local vasculature is vital for the formation of new bone during normal bone maintenance and following fracture. Therefore, the formation of blood vessels was measured with CD31 immunohistochemistry, and revealed that angiogenesis was significantly reduced in PS scaffolds compared to HS scaffolds (FIG. 24C).

The disclosure is predicated on our discovery that the disorder of critical illness [MeSH Descriptor C23.550.291.625] correlates with increased osteoclast formation from circulating precursors in the blood, osteoclast maturation and osteoclast bone resorption activity in comparison to healthy patients, resulting in complications, such as, “Osteopenia” (ICD-10 M85.8, ICD-9 733.90, DiseasesDB 29870 or MeSH D001851) or leading to the indication Critical Illness Bone Atrophy, Metabolic Bone Disease Following ICU Admission, Critical Illness Related Metabolic Bone Disease, Osteoporosis Secondary to ICU Admission with more extreme bone loss, which increases the risk of critically ill patients for fractures, such as, stress fractures (ICD-10-CM 733.93 Stress fracture of tibia or fibula, 733.94 Stress fracture of the metatarsals, 733.95 Stress fracture of other bone, 733.96 Stress fracture of femoral neck, 733.97 Stress fracture of shaft of femur and/or 733.98 Stress fracture of pelvis) and that usually needs that usually needs post-ICU ambulatory care. The applicants found that trabecular bone mineral content and density were reduced in critically ill rabbits, coinciding with low serum levels of ionized calcium and osteocalcin. Moreover, it was found that increased number of PBMC osteoclast precursors in the blood of critically ill humans resulted in increased in vitro osteoclast formation, which was further potentiated by the addition of serum from critically ill patients. Unexpectedly, neutralizing the inflammatory cytokines TNF-α and IL-6 in patient serum further increased osteoclast formation in patient PBMC cultures.

In an in vitro model of osteoblast formation, hPDC differentiation was unaffected by critically ill patient serum, although a reduction in VEGF-R1 gene and protein expression was observed. In vivo, bone formation was reduced in scaffolds containing patient serum-treated hPDCs, possibly due to an increase in osteoclastic activity together with a reduction in vascularization. As previously reported, the rabbit model of critical illness mirrors many of the metabolic and endocrine changes observed in critically ill patients (Weekers F, Giulietti A P, Michalaki M, Coopmans W, Van Herck E, Mathieu C, et al. Metabolic, endocrine, and immune effects of stress hyperglycemia in a rabbit model of prolonged critical illness. Endocrinology. 2003; 144(12):5329-38.). In the current study, critically ill rabbits displayed low levels of ionized calcium throughout the 7-day period of illness. During critical illness in humans, abnormalities in ionized calcium levels are common, with 90% of patients displaying mild hypocalcaemia, although hypercalcemia has also been reported in the protracted phase of illness (Egi M, Kim I, Nichol A, Stachowski E, French C J, Hart G K, et al. Ionized calcium concentration and outcome in critical illness. Crit. Care Med. 39(2):314-21). Ostensibly, these alterations may be linked with decreased levels of vitamin D, which have previously been reported during critical illness (Van den Berghe G, Van Roosbroeck D, Vanhove P, Wouters P J, De Pourcq L, Bouillon R. Bone turnover in prolonged critical illness: effect of vitamin D. J Clin Endocrinol Metab. 2003; 88(10):4623-32). Also, the observed reduction in serum osteocalcin in critically ill rabbits after 4 days is in line with findings in critically ill humans (Van den Berghe G, Van Roosbroeck D, Vanhove P, Wouters P J, De Pourcq L, Bouillon R. Bone turnover in prolonged critical illness: effect of vitamin D. J Clin Endocrinol Metab. 2003; 88(10):4623-32), which suggests that bone formation may also be reduced during critical illness. This was confirmed by the applicant's study, in which trabecular BMC and BMD were found reduced by 30.6% and 28.8%, respectively. In another rabbit model of osteoporosis evoked by methylprednisolone, trabecular BMD was reduced by 36% only after 10 weeks of treatment (Baofeng L, Zhi Y, Bei C, Guolin M, Qingshui Y, Jian L. Characterization of a rabbit osteoporosis model induced by ovariectomy and glucocorticoid. Acta Orthop. 81(3):396-401. PMCID: 2876847), and, therefore, the observation of similarly reduced trabecular bone after only 7 days of critical illness is remarkable. A severe catabolic state is observed during critical illness (Weekers F, Giulietti A P, Michalaki M, Coopmans W, Van Herck E, Mathieu C, et al. Metabolic, endocrine, and immune effects of stress hyperglycemia in a rabbit model of prolonged critical illness. Endocrinology. 2003; 144(12):5329-38).

A direct association was observed between the extreme catabolic state experienced during critical illness and subsequent skeletal morbidity. This is confirmed by a recent retrospective longitudinal case-cohort study by Orford and colleagues revealing an increased risk of fragility fractures over an 8-year period in elderly female survivors of critical illness (Orford N R, Saunders K, Merriman E, Henry M, Pasco J, Stow P, et al. Skeletal morbidity among survivors of critical illness. Crit. Care Med. 2011; 39(6):1295-300).

Furthermore, we revealed by FACS analysis carried out on fresh human peripheral blood samples from critically ill patients a significant increase in circulating early osteoclast precursors, demonstrating that, even prior to in vitro manipulation, critically ill patient PBMCs display increased osteoclastogenic potential (development of osteoclasts). In addition to classical metabolic bone diseases, such as, postmenopausal osteoporosis (D'Amelio P, Grimaldi A, Cristofaro M A, Ravazzoli M, Molinatti P A, Pescarmona G P, et al. Alendronate reduces osteoclast precursors in osteoporosis. Osteoporos Int. 21(10):1741-50), increased circulating osteoclast precursors have been suggested to lead to skeletal morbidity in disorders including phenylketonuria (Roato I, Porta F, Mussa A, D'Amico L, Fiore L, Garelli D, et al. Bone impairment in phenylketonuria is characterized by circulating osteoclast precursors and activated T cell increase. PLoS One. 5(11):e14167. PMCID: 2994752), multiple myeloma (Roato I, Grano M, Brunetti G, Colucci S, Mussa A, Bertetto O, et al. Mechanisms of spontaneous osteoclastogenesis in cancer with bone involvement. FASEB J. 2005; 19(2):228-30), and chronic liver disease (Olivier B J, Schoenmaker T, Mebius R E, Everts V, Mulder C J, van Nieuwkerk K M, et al. Increased osteoclast formation and activity by peripheral blood mononuclear cells in chronic liver disease patients with osteopenia. Hepatology. 2008; 47(1):259-67).

This increase in osteoclast precursors is often characterized by an increase in osteoclast formation and activity, which can be recapitulated in vitro through the isolation and differentiation of PBMCs into osteoclasts, with the addition of osteoclastogenic factors, such as, RANKL and M-CSF. We found that, PBMCs isolated from critically ill patients displayed a significant increase in differentiation into osteoclasts compared to healthy controls.

In cultures of PBMCs from critically ill patients, we observed osteoclast formation both in the presence and absence of RANKL and M-CSF. However, these mature, multi-nuclear osteoclasts were only able to actively resorb hydroxyapatite in the presence of osteoclastogenic factors.

In the current study, osteoclast formation and activity were further increased in patient PBMC cultures upon the addition of 10% PS (patient serum), whereas no increase was observed with HS (Healthy serum), or in healthy PBMC cultures treated with PS. This suggested that factors present in the PS were reacting with unique factors present in patient PBMCs to promote osteoclastogenesis. Inflammatory cytokines have previously been shown to promote the formation of osteoclasts in vitro and in vivo (D'Amelio P, Roato I, D'Amico L, Veneziano L, Suman E, Sassi F, et al. Bone and bone marrow pro-osteoclastogenic cytokines are up-regulated in osteoporosis fragility fractures. Osteoporos Int., Roato I, D'Amelio P, Gorassini E, Grimaldi A, Bonello L, Fiori C, et al. Osteoclasts are active in bone forming metastases of prostate cancer patients. PLoS One. 2008; 3(11):e3627. PMCID: 2574033, Boyce B F, Li P, Yao Z, Zhang Q, Badell I R, Schwarz E M, et al. TNF-alpha and pathologic bone resorption. Keio J. Med. 2005; 54(3):127-31), and have been directly related to fragility fractures in postmenopausal females (D'Amelio P, Roato I, D'Amico L, Veneziano L, Suman E, Sassi F, et al. Bone and bone marrow pro-osteoclastogenic cytokines are up-regulated in osteoporosis fragility fractures. Osteoporos Int.). However, neutralizing antibodies for both TNF-α and IL-6 further increased osteoclast formation in patient PBMC cultures with 10% PS. Resorption is thought to be mediated largely by the increased local production of pro-inflammatory cytokines, such as, TNF-α, which is thought to induce resorption indirectly by affecting the production of RANKL and/or its soluble decoy receptor, osteoprotegerin, by osteoblasts or directly by enhancing the activity of osteoclasts (Boyce B F, Xing L. Functions of RANKL/RANK/OPG in bone modeling and remodeling. Archives of Biochemistry and Biophysics. 2008; 473(2):139-46, Otero J E, Dai S, Alhawagri M A, Darwech I, Abu-Amer Y. IKKbeta activation is sufficient for RANK-independent osteoclast differentiation and osteolysis. J Bone Miner Res. 25(6):1282-94. PMCID: 3153134.).

In addition to an increased risk of fractures during rehabilitation, such bone hyperresorption could also predispose critically ill patients to impaired healing of traumatic and surgical bone lesions. In particular, patients in the ICU are at risk of slow or incomplete healing of the sternum following sternotomy for cardiac surgery (Fedak P W, Kasatkin A. Enhancing Sternal Closure Using Kryptonite Bone Adhesive: Technical Report. Surg Innov. 2011; Epub ahead of print). In order to investigate the effect of critical illness on bone formation during skeletal repair, critically ill patient serum was applied to the cells presently considered essential for fracture healing, namely human periosteal derived cells (hPDCs) (De Bari C, Dell'Accio F, Vanlauwe J, Eyckmans J, Khan I M, Archer C W, et al. Mesenchymal multipotency of adult human periosteal cells demonstrated by single-cell lineage analysis. Arthritis Rheum. 2006; 54(4):1209-21, Roberts S J, Geris L, Kerckhofs G, Desmet E, Schrooten J, Luyten F P. The combined bone forming capacity of human periosteal derived cells and calcium phosphates. Biomaterials. 32(19):4393-405, Colnot C. Skeletal cell fate decisions within periosteum and bone marrow during bone regeneration. J Bone Miner Res. 2009; 24(2):274-82).

Furthermore, the impact of prolonged critically ill patient serum on the differentiation capacity of hPDCs was evaluated by mineralization of the extracellular matrix with calcium deposition. Although an increase in mineralization was observed in all differentiated conditions compared to non-differentiated cells, no differences were observed between HS or PS. Similarly, gene expression of RUNX2, COL1A1 and ALP revealed no differences in expression between HS and PS conditions, although a reduction in RUNX2 and COL1A1 expression was observed in both human serums compared to the standard FBS condition. Under specific conditions, hPDCs are known to be capable of differentiation into the chondrocyte, osteoblast, adipocyte, and skeletal myocyte lineages in vitro and in vivo. However, in the current study carried out by applicants, hPDCs were directed towards the osteoblast lineage in order to focus directly on the effect of critical illness during bone formation. Although these findings suggest that in vitro, factors, such as, inflammatory cytokines present in critically ill PS do not have an effect on osteogenic differentiation, the fact that the expression of VEGF-R1 at both the gene and protein level was significantly reduced with PS suggests that vascularization may be inhibited during skeletal healing and bone formation in critical illness.

This hypothesis was corroborated by the in vivo model of bone formation during critical illness, where a reduction in bone formation in PS-coated NUOSS™ scaffolds implanted in NMRI-nu/nu mice correlated with a significant reduction in vascularization, along with an increase in osteoclast activity. This finding directly supports the hypothesis that hypoxia is a major risk factor for impaired fracture healing, such as, evidenced by sternal healing problems after internal mammary artery harvesting for coronary bypass surgery. Due to the fact that the majority of studies aimed at enhancing sternal closure utilize mechanical interventions, such as, wire stabilization (Iwakura A, Tabata Y, Nishimura K, Nakamura T, Shimizu Y, Fujita M, et al. Basic fibroblast growth factor may improve devascularized sternal healing. Ann Thorac Surg. 2000; 70(3):824-8, Iwakura A, Tabata Y, Koyama T, Doi K, Nishimura K, Kataoka K, et al. Gelatin sheet incorporating basic fibroblast growth factor enhances sternal healing after harvesting bilateral internal thoracic arteries. J Thorac Cardiovasc Surg. 2003; 126(4):1113-20), the finding that vascularization and bone formation in the model was inhibited by critically ill PS suggests that a biological approach to enhance fracture healing aimed at increasing vascularization also warrants further investigation.

An autophagy activator compound for use in treating or preventing bone degenerative disorder in a mammalian subject can have different structures. The autophagy activator compound can be a compound of the group consisting of Rapamycin, Nigericin, Wiskostatin, Fluspiriline, Niguldipine, Trifluoperazine, Nicardipine and Penitrem A (Tremortin). An autophagy activator compound for use in treating or preventing bone degenerative disorder in a mammalian subject can have different structures. The autophagy activator compound can be a compound of the group consisting of Loperamide, Amiodarone, Niguldipine, Pimozide, Nicardipine, Penitrem A, Fluspirilene, Trifluoperazine, and pharmaceutically acceptable salts thereof. Trehalose, a non-reducing disaccharide present in many non-mammalian species, including bacteria, yeast, fungi, insects, invertebrates (natural hemolymph sugar of invertebrates), and plants is an autophagy inducer according to the instant disclosure for use in treating or preventing bone degenerative disorder in a mammalian subject. Trehalose is also known as mycose or tremalose, is a natural alpha-linked disaccharide formed by an α,α-1,1-glucoside bond between two α-glucose units.

Aspects of the disclosure are the following. Loperamide for use in treating or preventing bone degenerative disorder in a mammalian subject. Amiodarone for use in treating or preventing bone degenerative disorder in a mammalian subject. Niguldipine for use in treating or preventing bone degenerative disorder in a mammalian subject. Pimozide for use in treating or preventing bone degenerative disorder in a mammalian subject. Nicardipine for use in treating or preventing bone degenerative disorder in a mammalian subject. Penitrem A for use in treating or preventing bone degenerative disorder in a mammalian subject. Fluspirilene for use in treating or preventing bone degenerative disorder in a mammalian subject. Trifluoperazine for use in treating or preventing bone degenerative disorder in a mammalian subject. Trehalose for use in treating or preventing bone degenerative disorder in a mammalian subject. A particular aspect of the disclosure features a kit that includes: (i) a pharmaceutical composition comprising an autophagy inducing compound and (ii) instructions for administering the composition to a subject for the treatment of the bone degenerative disorder. Moreover, in a particular embodiment, the autophagy inducing compound is administered to reach plasma concentrations of (about) 0.1 nM to (about) 150 nM. In a preferred embodiment of this aspect, the autophagy inducing compound is administered at a dosis to reach plasma concentration of (about) 3.0 nM to (about) 9.0 nM.

Also described is a pharmaceutical composition comprising an autophagy inducing compound in an amount effective for treating a bone degenerative disorder, wherein the compound is selected from the group consisting of: (a) Loperamide; (b) Amiodarone; (c) Niguldipine; (d) Pimozide; (e) Nicardipine; (f) Penitrem A; (g) Fluspirilene, (h) Trifluoperazine and (i) trehalose or invention concerns a pharmaceutical composition comprising an autophagy inducing compound, wherein the compound is selected from the group consisting of: (a) Loperamide; (b) Amiodarone; (c) Niguldipine; (d) Pimozide; (e) Nicardipine; (f) Penitrem A; (g) Fluspirilene, (h) Trifluoperazine and (i) trehalose for use in treating or preventing bone degenerative disorder in a mammalian subject. Such pharmaceutical compositions can be as described herein, wherein the composition further comprises a pharmaceutically acceptable carrier. The bone degenerative disorder can be to inhibit increased bone resorption disorder, to inhibit osteoclast activity, to inhibit formation of osteoclasts (TRAP-positive multinuclear cells) from blood progenitor cells, or to activate bone formation. The bone degenerative disorders, can be bone degenerative disorder caused by acute critical illness or prolonged critical illness, or the bone degenerative disorder is caused by (parenteral) nutrition-induced suppression of autophagy, or the bone degenerative disorder is caused by mTOR activation, or the bone degenerative disorders is hyperresorption of bone, or the bone degenerative disorders is an imbalance in the regulation of bone resorption and bone formation resulting in metabolic bone diseases, such as, osteoporosis, or the bone degenerative disorders is elderly osteoporosis, or the bone degenerative disorders is osteoporosis or the bone degenerative disorders is pathological increased bone resorption and pathological decreased bone formation, or the bone degenerative disorders is disrupted calcium and bone metabolism by acute critical illness and/or prolonged critical illness, or the bone degenerative disorders is caused by enhanced osteoclast formation from circulating precursors in the blood during critical illness, or the bone degenerative disorders is increased osteoclast formation from circulating precursors leading to osteoporosis, or the bone degenerative disorders is increased osteoclast formation due to a increased cytokine production disorder, or the bone degenerative disorders is increased osteoclast formation due to hormonal disorder, or the bone degenerative disorders is increased osteoclast formation due to Vitamin D deficiency, or the bone degenerative disorders is increased osteoclast formation due to glucocortoids (pre)treatment, or the bone degenerative disorders is increased osteoclast formation due to heparinoids (pre)treatment, or the bone degenerative disorders is increased osteoclast formation due to autophagy deficiency disorder, or the bone degenerative disorders is osteoclast activity increase due to acute critical illness and/or prolonged critical illness, or the bone degenerative disorders is osteoclast activity increase due to increased cytokine production disorder or the bone degenerative disorders is osteoclast activity increase due to hormonal disorder.

Also described is a pharmaceutical composition comprising an autophagy inducing compound in an amount effective for treating a bone degenerative disorder, wherein the compound is selected from the group consisting of: (a) Loperamide; (b) Amiodarone; (c) Niguldipine; (d) Pimozide; (e) Nicardipine; (f) Penitrem A; (g) Fluspirilene, (h) Trifluoperazine and (i) trehalose or invention concerns a pharmaceutical composition comprising an autophagy inducing compound, wherein the compound is selected from the group consisting of (a) Loperamide; (b) Amiodarone; (c) Niguldipine; (d) Pimozide; (e) Nicardipine; (f) Penitrem A; (g) Fluspirilene, (h) Trifluoperazine and (i) trehalose for use in treating or preventing bone degenerative disorder in a mammalian subject. Such a pharmaceutical composition can be as described herein, wherein the composition further comprises a pharmaceutically acceptable carrier. The bone degenerative disorder can be to inhibit increased bone resorption disorder, to inhibit osteoclast activity, to inhibit formation of osteoclasts (TRAP-positive multinuclear cells) from blood progenitor cells, to activate bone formation. The bone degenerative disorders is bone metabolism disorder, can be bone degenerative disorder caused by acute critical illness or prolonged critical illness, or the bone degenerative disorder is caused by (parenteral) nutrition-induced suppression of autophagy or the bone degenerative disorder is caused by mTOR activation, or the bone degenerative disorders is hyperresorption of bone or the bone degenerative disorders is an imbalance in the regulation of bone resorption and bone formation resulting in metabolic bone diseases, such as, osteoporosis or the bone degenerative disorders is elderly osteoporosis or the bone degenerative disorders is osteoporosis or the bone degenerative disorders is pathological increased bone resorption and pathological decreased bone formation, or the bone degenerative disorders is disrupted calcium and bone metabolism by acute critical illness and/or prolonged critical illness, or the bone degenerative disorders is caused by enhanced osteoclast formation from circulating precursors in the blood during critical illness, or the bone degenerative disorders is increased osteoclast formation from circulating precursors leading to osteoporosis, or the bone degenerative disorders is increased osteoclast formation due to a increased cytokine production disorder, or the bone degenerative disorders is increased osteoclast formation due to hormonal disorder, or the bone degenerative disorders is increased osteoclast formation due to Vitamin D deficiency, or the bone degenerative disorders is increased osteoclast formation due to glucocortoids (pre)treatment, or the bone degenerative disorders is increased osteoclast formation due to heparinoids (pre)treatment, or the bone degenerative disorders is increased osteoclast formation due to autophagy deficiency disorder, or the bone degenerative disorders is osteoclast activity increase due to acute critical illness and/or prolonged critical illness, or the bone degenerative disorders is osteoclast activity increase due to increased cytokine production disorder or the bone degenerative disorders is osteoclast activity increase due to hormonal disorder.

Also described is a pharmaceutical composition for use in treating or preventing bone degenerative disorder in a mammalian subject the pharmaceutical composition comprising an autophagy inducing compound in an amount effective for treating a bone degenerative disorder, wherein the compound is at least one compound selected from the group consisting of:

(a) compounds of formula (I):

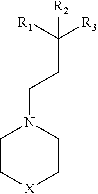

wherein X is selected from CR4R5 and NR6; R1 is selected from hydrogen, C1-6 alkyl and phenyl, wherein the alkyl and phenyl are substituted with 0 or 1 halogen; R2 is selected from hydrogen, C1-6 alkyl and phenyl, wherein the alkyl and phenyl are substituted with 0 or 1 halogen; R3 is selected from

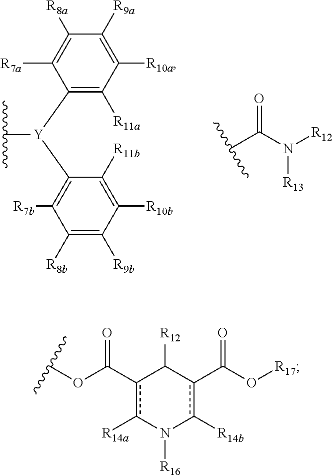

R4 is selected from hydrogen, hydroxyl, C1-6 alkyl and phenyl;
R5 is selected from C1-6 alkyl and phenyl, halophenyl, benzimidazole, dihydrobenzimidazole, benzimidazolone; optionally R4 and R5 are taken together to form a 5 or 6 membered heterocycloalkyl comprising two nitrogen atoms, wherein the heterocycloalkyl is substituted with 1, 2 or 3 substituents selected from the group consisting of C1-6 alkyl, phenyl, and ═O; R6 is selected from hydrogen and C1-6 alkyl; R7a, R8a, R9a, R10a, R11a, R7b, R8b, R9b, R10b, and R11b are each independently selected from hydrogen, hydroxyl, halogen and C1-6 haloalkyl; optionally R11a and R11b are taken together to form a heterocycle of the following structure:

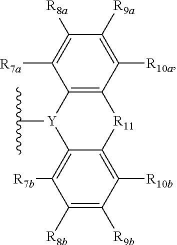

wherein R11 is selected from CH2, NH, O and S; R12 and R13 are each independently selected from hydrogen and C1-6 alkyl; R14a and R14b are each independently selected from hydrogen and C1-6 alkyl; R15 is selected from phenyl substituted with 0 or 1 halogen or nitro; R16 is selected from hydrogen and C1-6 alkyl; Y is N or CH; and pharmaceutically acceptable salts thereof; (b) compounds of formula (II):

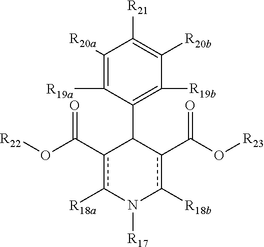

wherein R17 is selected from hydrogen and C1-6 alkyl; R18a and R18b, are each independently selected from hydrogen and C1-6 alkyl; R19a, R19b, R20a, R20b, and R21 are each independently selected from hydrogen, halogen and nitro; R22 is selected from hydrogen and C1-6 alkyl; R23 is selected from —(CH2)nNR24aR24b and —(CH2)nR24a; R24a and R24b are each independently selected from C1-6 alkyl and phenyl, wherein the alkyl is substituted with 0 or 1 phenyl substituents; optionally R24a and R24b are taken together with the nitrogen to which they are attached to form a piperidine which is substituted with 0, 1 or 2 phenyl substituents; n is a positive integer from 2 to 4; and pharmaceutically acceptable salts thereof
(c) compounds of formula (III):

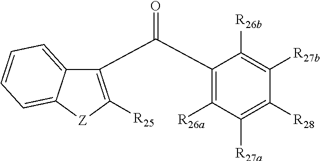

wherein R25 is selected from hydrogen and C1-6 alkyl; R26a, R26b, R27a, and R27b are each independently selected from hydrogen, halogen and C1-6 alkyl; R28 is selected from —O(CH2)mNR29aR29b and —NH(CH2)mNR29aR29b; R29a and R29b are each independently selected from hydrogen and C1-6 alkyl; Z is O, S or NH; m is a positive integer from 1 to 3; and pharmaceutically acceptable salts thereof
(d) compounds of formula (IV):

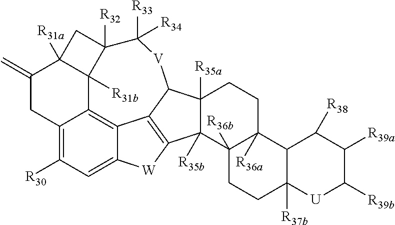

wherein R30 is selected from hydrogen, C1-6 alkyl and halogen; R31a and R31b are each independently selected from hydrogen, hydroxyl and C1-6 alkyl; R32 is selected from hydrogen, hydroxyl and C1-6 alkyl; R33 and R34 are each independently selected from hydrogen and C1-6 alkyl; R35a and R35b are each independently selected from hydrogen, hydroxyl and C1-6 alkyl; R36a and R36b are each independently selected from hydrogen, hydroxyl and C1-6 alkyl; R37a and R37b are each independently selected from hydrogen, hydroxyl and C1-6 alkyl; R38 is selected from hydrogen, hydroxyl and C1-6 alkyl; optionally R37a and R38 are taken together to form a three membered heterocycle of the formula:

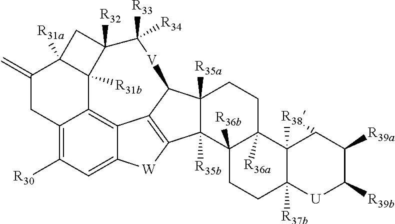

wherein R38′ is O, S or NH; R39a is selected from hydrogen, hydroxyl and C1-6 alkyl; R39b is selected from hydrogen, hydroxyl, C1-6 alkyl and C2-6 alkenyl; U, V and W are each independently selected from O, S, and NH; and pharmaceutically acceptable salts thereof.

Suitable Rapamycin analogues for the disclosure are of the group of the Rapamycin derivatives, such as, Everolimus, Temsirolimus, 40-O-(2-hydroxyethyl)-rapamycin, and/or 32-deoxorapamycin, and/or 16-pent-2-ynyloxy-32-deoxorapamycin, and/or 16-pent-2-ynyloxy-32 (S or R)-dihydro-rapamycin, and/or 16-pent-2-ynyloxy-32 (S or R)-dihydro-40-O-(2-hydroxyethyl)-rapamycin, and/or 40-[3-hydroxy-2-(hydroxy-methyl)-2-methylpropanoate]-rapamycin (also known as CCI779) and/or] 40-epi-(tetrazolyl)-rapamycin (also known as ABT578), and/or the so-called rapalogs, e.g., as disclosed in WO9802441, WO114387 and WO364383, AP23573, AP23464; AP23675 or AP23841, e.g., AP23573, and/or compounds disclosed under the name TAFA-93, and/or compounds disclosed under the name biolimus. Other for invention preferred rapamycin derivative is selected from the group consisting of 40-O-(2-hydroxyethyl)-rapamycin (also known as everolimus), and/or 32-deoxorapamycin, and/or 16-pent-2-ynyloxy-32-deoxorapamycin, and/or 16-pent-2-ynyloxy-32 (S or R)-dihydro-rapamycin, and/or 16-pent-2-ynyloxy-32 (S or R)-dihydro-40-O-(2-hydroxyethyl)-rapamycin, and/or 40-[3-hydroxy-2-(hydroxy-methyl)-2-methylpropanoate]-rapamycin (also known as CC1779 or temsirolimus) and/or 40-epi-(tetrazolyl)-rapamycin (also known as ABT578), and/or AP23573, such as, 40-O-(2-hydroxyethyl)-rapamycin.

Suitable phenothiazine derivatives for invention are, for example, compounds chosen from quinacrine, imipramine, carbamazepine, phenazine, phenothiazine, promazine, chloropromazine, haloperidol, clozapine, 2-chlorophenothiazine, promethazine (10-(2-dimethylaminopropyl)phenothiazine), chloroprothixen, and acepromazine.

Another mTOR dependent autophagy inducer suitable for the medicament and treatment of the invention is Resveratrol and derivatives thereof as described in WO2005102298 or WO2007096078. Resveratrol is an autophagy induced via the mTOR-Rictor survival pathway. Resveratrol at lower doses induces autophagy while higher doses are known to attenuate autophagy. The activation of mammalian target of rapamycin (mTOR) is differentially regulated by low-dose resveratrol, i.e., the phosphorylation of mTOR at serine 2448 is inhibited, whereas the phosphorylation of mTOR at serine 2481 is increased, which is attenuated with a higher dose of resveratrol. Low-dose resveratrol significantly induces the expression of Rictor, a component of mTOR complex 2, and activated its downstream survival kinase Akt (Ser 473).

The term “resveratrol, a derivative, metabolite or analogue thereof” concerns compounds encompassed by the general formula I

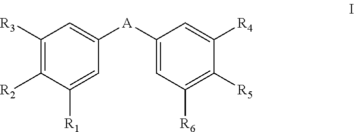

wherein A denotes a carbon-carbon single or double bound, and the latter hereby may be trans or cis, and R1, R2, R3, R4, R5 and R6, independently from each other denote hydrogen, hydroxyl, etherified hydroxyl or esterified hydroxy groups.

Another mTOR dependent autophagy inducer suitable for the medicament and treatment of the invention is fenofibrate. Fenofibrate functions as autophagy activator by activating AMPK. AMPK switches on p53-dependent cell cycle metabolic check point and autophagy. AMPK antagonizes this Akt-induced mTOR activation. AMP-dependent protein kinase (AMPK) plays an integral role in the response to starvation by sensing the rise in AMP/ATP ratio and switching off the ATP-consuming anabolic processes, such as, protein and lipid synthesis or DNA replication. AMPK can induce several rescue pathways, which enhance cell survival during glucose deprivation. One of them includes p53-dependent check point, which promotes autophagy.

Other compounds suitable for the medicament and treatment of the invention are DNA methylation inhibitors and, in particular, the DNA methylation inhibitor, 5-azacytidine.

Also described is an autophagy inducing compound for use in treating or preventing bone degenerative disorder in a mammalian, wherein the compound is at least one compound selected from the group consisting of:

(a) compounds of formula (I):

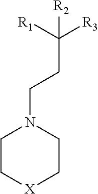

wherein X is selected from CR4R5 and NR6; R1 is selected from hydrogen, C1-6 alkyl and phenyl, wherein the alkyl and phenyl are substituted with 0 or 1 halogen; R2 is selected from hydrogen, C1-6 alkyl and phenyl, wherein the alkyl and phenyl are substituted with 0 or 1 halogen; R3 is selected from

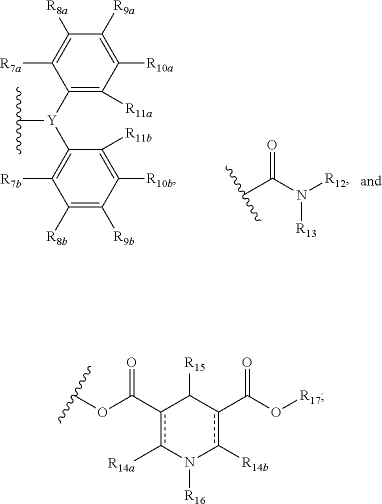

R4 is selected from hydrogen, hydroxyl, C1-6 alkyl and phenyl;
R5 is selected from C1-6 alkyl and phenyl, halophenyl, benzimidazole, dihydrobenzimidazole, benzimidazolone; optionally R4 and R5 are taken together to form a 5 or 6 membered heterocycloalkyl comprising two nitrogen atoms, wherein the heterocycloalkyl is substituted with 1, 2 or 3 substituents selected from the group consisting of C1-6 alkyl, phenyl, and ═O;
R6 is selected from hydrogen and C1-6 alkyl; R7a, R8a, R9a, R10a, R11a, R7b, R8b, R9b, R10b, and R11b are each independently selected from hydrogen, hydroxyl, halogen and C1-6 haloalkyl; optionally R11a and R11b are taken together to foam a heterocycle of the following structure:

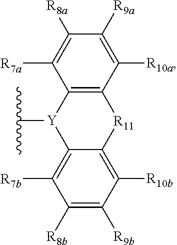

wherein R11 is selected from CH2, NH, O and S; R12 and R13 are each independently selected from hydrogen and C1-6 alkyl; R14a and R14b are each independently selected from hydrogen and C1-6 alkyl; R15 is selected from phenyl substituted with 0 or 1 halogen or nitro; R16 is selected from hydrogen and C1-6 alkyl; Y is N or CH; and pharmaceutically acceptable salts thereof;
(b) compounds of formula (II):

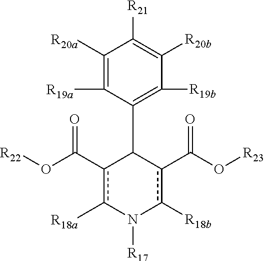

wherein R17 is selected from hydrogen and C1-6 alkyl; R18a and R18b, are each independently selected from hydrogen and C1-6 alkyl; R19a, R19b, R20a, R20b, and R21 are each independently selected from hydrogen, halogen and nitro; R22 is selected from hydrogen and C1-6 alkyl; R23 is selected from —(CH2)nNR24aR24b and —(CH2)nR24a; R24a and R24b are each independently selected from C1-6 alkyl and phenyl, wherein the alkyl is substituted with 0 or 1 phenyl substituents; optionally R24a and R24b are taken together with the nitrogen to which they are attached to form a piperidine, which is substituted with 0, 1 or 2 phenyl substituents; n is a positive integer from 2 to 4; and pharmaceutically acceptable salts thereof
(c) compounds of formula (III):

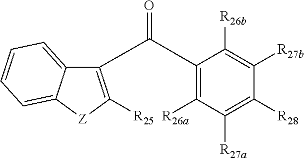

wherein R25 is selected from hydrogen and C1-6 alkyl; R26a, R26b, R27a, and R27b are each independently selected from hydrogen, halogen and C1-6 alkyl; R28 is selected from —O(CH2)mNR29aR29b and —NH(CH2)mNR29aR29b; R29a and R29b are each independently selected from hydrogen and C1-6 alkyl; Z is O, S or NH; m is a positive integer from 1 to 3; and pharmaceutically acceptable salts thereof
(d) compounds of formula (IV):

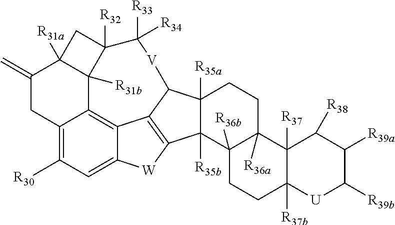

wherein R30 is selected from hydrogen, C1-6 alkyl and halogen; R31a and R31b are each independently selected from hydrogen, hydroxyl and C1-6 alkyl; R32 is selected from hydrogen, hydroxyl and C1-6 alkyl; R33 and R34 are each independently selected from hydrogen and C1-6 alkyl; R35a and R35b are each independently selected from hydrogen, hydroxyl and C1-6 alkyl; R36a and R36b are each independently selected from hydrogen, hydroxyl and C1-6 alkyl; R37a and R37b are each independently selected from hydrogen, hydroxyl and C1-6 alkyl; R38 is selected from hydrogen, hydroxyl and C1-6 alkyl; optionally R37a and R38 are taken together to form a three membered heterocycle of the formula:

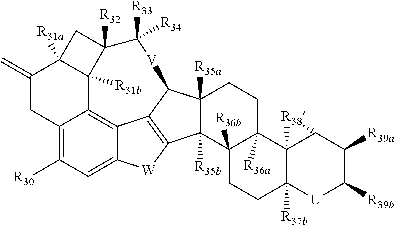

wherein R38′ is O, S or NH; R39a is selected from hydrogen, hydroxyl and C1-6 alkyl; R39b is selected from hydrogen, hydroxyl, C1-6 alkyl and C2-6 alkenyl; U, V and W are each independently selected from O, S, and NH; and pharmaceutically acceptable salts thereof.

The bone degenerative disorder can be to inhibit increased bone resorption disorder, to inhibit osteoclast activity, to inhibit formation of osteoclasts (TRAP-positive multinuclear cells) from blood progenitor cells, to activate bone formation. The bone degenerative disorders is bone metabolism disorder, can be bone degenerative disorder caused by acute critical illness or prolonged critical illness, or the bone degenerative disorder is caused by (parenteral) nutrition-induced suppression of autophagy or the bone degenerative disorder is caused by mTOR activation, or the bone degenerative disorders is hyperresorption of bone or the bone degenerative disorders is an imbalance in the regulation of bone resorption and bone formation results in metabolic bone diseases, such as, osteoporosis or the bone degenerative disorders is elderly osteoporosis or the bone degenerative disorders is osteoporosis or the bone degenerative disorders is pathological increased bone resorption and pathological decreased bone formation, or the bone degenerative disorders is disrupted calcium and bone metabolism by acute critical illness and/or prolonged critical illness, or the bone degenerative disorders is caused by enhanced osteoclast formation from circulating precursors in the blood during critical illness, or the bone degenerative disorders is increased osteoclast formation from circulating precursors leading to osteoporosis, or the bone degenerative disorders is increased osteoclast formation due to a increased cytokine production disorder, or the bone degenerative disorders is increased osteoclast formation due to hormonal disorder, or the bone degenerative disorders is increased osteoclast formation due to Vitamin D deficiency, or the bone degenerative disorders is increased osteoclast formation due to glucocortoids (pre)treatment, or the bone degenerative disorders is increased osteoclast formation due to heparinoids (pre)treatment, or the bone degenerative disorders is increased osteoclast formation due to autophagy deficiency disorder, or the bone degenerative disorders is osteoclast activity increase due to acute critical illness and/or prolonged critical illness, or the bone degenerative disorders is osteoclast activity increase due to increased cytokine production disorder or the bone degenerative disorders is osteoclast activity increase due to hormonal disorder.

Also described is the use of such a compound for use in a treatment to activate bone formation in a subject, for use in a treatment to treat a bone metabolism disorder, for use in a treatment to treat a bone metabolism disorder caused by acute critical illness or prolonged critical illness, for use in a treatment to treat a bone metabolism disorder caused by (parenteral) nutrition-induced suppression of autophagy, for use in a treatment to treat a bone metabolism disorder caused by mTOR activation, for use in a treatment to treat hyperresorption of bone, for use in a treatment to treat an imbalance in the regulation of bone resorption and bone formation resulting in metabolic bone diseases, such as, osteoporosis, for use in a treatment to treat elderly osteoporosis, for use in a treatment to treat osteoporosis, for use in a treatment of a patient with pathological increased bone resorption and pathological decreased bone formation, for use in a treatment of disrupted calcium and bone metabolism by acute critical illness and/or prolonged critical illness, for use in a treatment of disorders of bone metabolism of enhanced osteoclast formation from circulating precursors in the blood during critical illness, for use in a treatment of increased osteoclast formation from circulating precursors leading to osteoporosis, for use in a treatment of increased osteoclast formation due to an increased cytokine production disorder, for use in a treatment of increased osteoclast formation due to hormonal disorder, for use in a treatment of increased osteoclast formation due to Vitamin D deficiency, for use in a treatment of increased osteoclast formation due to glucocortoids (pre)treatment, for use in a treatment of increased osteoclast formation due to heparinoids (pre)treatment, for use in a treatment of increased osteoclast formation due to autophagy deficiency disorder, for use in a treatment to inhibit osteoclast activity increase due to acute critical illness and/or prolonged critical illness, for use in a treatment to inhibit osteoclast activity increase due to increased cytokine production disorder, for use in a treatment to inhibit osteoclast activity increase due to hormonal disorder, for use in a treatment to inhibit osteoclast activity increase due to Vitamin D deficiency, for use in a treatment to inhibit osteoclast activity increase due to glucocortoids (pre)treatment, for use in a treatment to inhibit osteoclast activity increase due to heparinoids (pre)treatment, for use in a treatment to inhibit osteoclast activity increase due to autophagy deficiency disorder or for use in a treatment to inhibit bone hyperresorption

Also described is a pharmaceutical composition comprising a pharmacologically acceptable amount of an autophagy inducing or autophagy inducing compound or an autophagy inducing or autophagy inducing compound analog, as described above, a pharmaceutically acceptable salt, solvate or isomer thereof, or combinations thereof. Also described is a pharmaceutical for use in a treatment to activate bone formation in a subject, for use in a treatment to treat a bone metabolism disorder, for use in a treatment to treat a bone metabolism disorder caused by acute critical illness or prolonged critical illness, for use in a treatment to treat a bone metabolism disorder caused by (parenteral) nutrition-induced suppression of autophagy, for use in a treatment to treat a bone metabolism disorder caused by mTOR activation, for use in a treatment to treat hyperresorption of bone, for use in a treatment to treat an imbalance in the regulation of bone resorption and bone formation resulting in metabolic bone diseases, such as, osteoporosis, for use in a treatment to treat elderly osteoporosis, for use in a treatment to treat osteoporosis, for use in a treatment of a patient with pathological increased bone resorption and pathological decreased bone formation, for use in a treatment of disrupted calcium and bone metabolism by acute critical illness and/or prolonged critical illness, for use in a treatment of disorders of bone metabolism of enhanced osteoclast formation from circulating precursors in the blood during critical illness, for use in a treatment of increased osteoclast formation from circulating precursors leading to osteoporosis, for use in a treatment of increased osteoclast formation due to a increased cytokine production disorder, for use in a treatment of increased osteoclast formation due to hormonal disorder, for use in a treatment of increased osteoclast formation due to Vitamin D deficiency, for use in a treatment of increased osteoclast formation due to glucocortoids (pre)treatment, for use in a treatment of increased osteoclast formation due to heparinoids (pre)treatment, for use in a treatment of increased osteoclast formation due to autophagy deficiency disorder, for use in a treatment to inhibit osteoclast activity increase due to acute critical illness and/or prolonged critical illness, for use in a treatment to inhibit osteoclast activity increase due to increased cytokine production disorder, for use in a treatment to inhibit osteoclast activity increase due to hormonal disorder, for use in a treatment to inhibit osteoclast activity increase due to Vitamin D deficiency, for use in a treatment to inhibit osteoclast activity increase due to glucocortoids (pre)treatment, for use in a treatment to inhibit osteoclast activity increase due to heparinoids (pre)treatment, for use in a treatment to inhibit osteoclast activity increase due to autophagy deficiency disorder or for use in a treatment to inhibit bone hyperresorption.

A further embodiment concerns a DNA methylation inhibitor for use in a method of inhibiting for suppressing osteoclastogenesis or osteoclast differentiation in a subject in need for a treatment of disorders of bone density (ICD-10-CM M80-M85). This DNA methylation inhibitor can be for use in a method of inhibiting for suppressing enhanced osteoclastogenesis or increased osteoclast differentiation in a subject in need for a treatment of a disorders of bone density (ICD-10-CM M80-M85). Moreover, this DNA methylation inhibitor can be for use in a method of inhibiting for suppressing critical illness [MeSH Descriptor: C23.550.291.625] enhanced osteoclastogenesis or increased osteoclast differentiation in a subject in need for a treatment of Critical Illness Related Metabolic Bone Disease or of critical illness induced Osteopenia [ICD-10 M85.8, ICD-9 733.90, DiseasesDB 29870 or MeSH D001851] secondary to ICU Admission. Suitable methylation inhibitor according to this invention is, for instance, a DNA methylation inhibitor selected from the group consisting of decitabine (or 5-aza-2′-deoxycytidine or) 5-azadC, azacitidine (or 5-azacytidine), vorinostat (ZOLINZA™), procainamide and derivatives thereof. It was surprisingly found that DNA methylation inhibitor according to invention can be delivered or administrated to subject in need thereof to reach very low nanomolar plasma concentration of a value of the range of 1 to 10 nM to suppress osteoclastogenesis or osteoclast differentiation.

When administered to a patient, an autophagy inducing or autophagy inducing compound or a methylation inhibitor is preferably administered as a component of a composition that optionally comprises a pharmaceutically acceptable carrier or vehicle. In one embodiment, these compositions are administered orally. In a preferred embodiment, the autophagy inducing or autophagy inducing compound of the invention is a component of a pharmaceutical composition that is administered intravenously.

A pharmaceutical composition comprising an autophagy inducing can be administered via one or more routes, such as, but not limited to, oral, intravenous infusion, subcutaneous injection, intramuscular, topical, depo injection, implantation, time-release mode, and intracavitary. The pharmaceutical composition is formulated to be compatible with its intended route of administration. Examples of routes of administration include parenteral, e.g., intravenous, intramuscular, intraperitoneal, intracapsular, intraspinal, intrasternal, intratumor, intranasal, epidural, intra-arterial, intraocular, intraorbital, intradermal, subcutaneous, oral (e.g., inhalation), transdermal (topical-particularly to the ears, nose, eyes, or skin), transmucosal (e.g., oral) nasal, rectal, intracerebral, intravaginal, sublingual, submucosal, and transdermal administration.

Administration can be via any route known to be effective by a physician of ordinary skill. Parenteral administration, i.e., not through the alimentary canal, can be performed by subcutaneous, intramuscular, intra-peritoneal, intratumoral, intradermal, intracapsular, intra-adipose, or intravenous injection of a dosage form into the body by means of a sterile syringe, optionally a pen-like syringe, or some other mechanical device, such as, an infusion pump. A further option is a composition that can be a powder or a liquid for the administration in the form of a nasal or pulmonary spray. As a still further option, the administration can be transdermally, e.g., from a patch. Compositions suitable for oral, buccal, rectal, or vaginal administration can also be provided. In a preferred embodiment, administration of the autophagy inducing or autophagy inducing compound of the invention is via an intravenous injection, e.g., an intravenous bolus injection or by gradual perfusion over time.

The autophagy inducing or autophagy inducing compound and the pharmaceutical composition of the invention can also be administered by a small bolus injection followed by a continuous infusion. One protocol for treatment with autophagy inducing or autophagy inducing compound or an autophagy inducing or autophagy inducing compound analog is as follows: (i) initial bolus injection over a period of 1-2 minutes; (ii) high level infusion for 1 hour; (2) low level maintenance infusion for 2-3 hours.

The whole of the dose of autophagy inducing or autophagy inducing compound required to achieve a protective effect could also be administered as one or more bolus injections e.g., ranging between 1-100 percent of the estimated required 24 h dose, or administered with a 50 cc syringe at a rate of 2 ml per hour.

The autophagy inducing or autophagy inducing compound and the pharmaceutical composition of the invention can also be administered by a small bolus injection followed by a continuous infusion. One protocol for treatment with autophagy inducing or autophagy inducing compound or a autophagy inducing or autophagy inducing compound analog is as follows: (i) initial bolus injection over a period of 1-2 minutes; (ii) high level infusion for 1 hour; (2) low level maintenance infusion for 2-3 hours.

The whole of the dose of autophagy inducing or autophagy inducing compound required to achieve a protective effect could also be administered as one or more bolus injections, e.g., administered with a 50 cc syringe at a rate of 2 ml over 1 hour.

In one embodiment, a pharmaceutical composition hereof is delivered by a controlled release system. For example, the pharmaceutical composition can be administered using intravenous infusion, an implantable osmotic pump, a transdermal patch, liposomes, or other modes of administration. In one embodiment, a pump can be used (See e.g., Langer, 1990, Science 249:1527-33; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, the compound can be delivered in a vesicle, in particular, a liposome (See e.g., Langer, 1990, Science 249:1527-33; Treat et al., 1989, in Liposomes in the Therapy of Infectious Disease and Cancer, Lopez-Berestein and Fidler (eds.), Liss, New York, pp. 353-65; Lopez-Berestein, ibid., pp. 317-27; International Patent Publication No. WO91/04014; U.S. Pat. No. 4,704,355). In another embodiment, polymeric materials can be used (See e.g., Medical Applications of Controlled Release, Langer and Wise (eds.), CRC Press: Boca Raton, Fla., 1974; Controlled Drug Bioavailability, Drug Product Design and Performance, Smolen and Ball (eds.), Wiley: New York (1984); Ranger and Peppas, 1953, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In yet another embodiment, a controlled release system can be placed in proximity of the target. For example, a micropump can deliver controlled doses directly into bone or adipose tissue, thereby, requiring only a fraction of the systemic dose (See e.g., Goodson, 1984, in Medical Applications of Controlled Release, vol. 2, pp. 115-138). In another example, a pharmaceutical composition hereof can be formulated with a hydrogel (See, e.g., U.S. Pat. Nos. 5,702,717; 6,117,949; 6,201,072).

In one embodiment, it may be desirable to administer the pharmaceutical composition hereof locally, i.e., to the area in need of treatment. Local administration can be achieved, for example, by local infusion during surgery, topical application (e.g., in conjunction with a wound dressing after surgery), injection, catheter, suppository, or implant. An implant can be of a porous, non-porous, or gelatinous material, including membranes, such as, sialastic membranes, or fibres.

In certain embodiments, it may be desirable to introduce the autophagy inducing or autophagy inducing compound into the central nervous system by any suitable route, including intraventricular, intrathecal, and epidural injection. Intraventricular injection may be facilitated by an intraventricular catheter, for example, attached to a reservoir, such as, an Ommaya reservoir.

Pulmonary administration can also be employed, e.g., by use of an inhaler or nebulizer, and formulation with an aerosolizing agent, or via perfusion in a fluorocarbon or synthetic pulmonary surfactant.

The skilled artisan can appreciate the specific advantages and disadvantages to be considered in choosing a mode of administration. Multiple modes of administration are encompassed by the invention. For example, an autophagy inducing or autophagy inducing compound hereof can be administered by subcutaneous injection, whereas another therapeutic agent can be administered by intravenous infusion. Moreover, administration of one or more species of autophagy inducing or autophagy inducing compounds, with or without other therapeutic agents, can occur simultaneously (i.e., co-administration) or sequentially. In another embodiment, the periods of administration of an autophagy inducing or autophagy inducing compound, with or without other therapeutic agents can overlap. For example, a autophagy inducing or autophagy inducing compound can be administered for 7 days and another therapeutic agent can be introduced beginning on the fifth day of autophagy inducing or autophagy inducing compound treatment. Treatment with the other therapeutic agent can continue beyond the 7-day autophagy inducing or autophagy inducing compound treatment.

A pharmaceutical composition of an autophagy inducing or autophagy inducing compound can be administered before, during, and/or after the administration of one or more therapeutic agents. In one embodiment, autophagy inducing or autophagy inducing compound can first be administered to stimulate the expression of insulin, which increases sensitivity to subsequent challenge with a therapeutic agent. In another embodiment, autophagy inducing or autophagy inducing compound can be administered after administration of a therapeutic agent. In yet another embodiment, there can be a period of overlap between the administration of the autophagy inducing or autophagy inducing compound and the administration of one or more therapeutic agents.

A pharmaceutical composition hereof can be administered in the morning, afternoon, evening, or diurnally. In one embodiment, the pharmaceutical composition is administered at particular phases of the circadian rhythm. In a specific embodiment, the pharmaceutical composition is administered in the morning. In another specific embodiment, the pharmaceutical composition is administered at an artificially induced circadian state.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (See e.g., U.S. Pat. No. 5,698,155).

Pharmaceutical compositions adapted for parenteral administration include, but are not limited to, aqueous and non-aqueous sterile injectable solutions or suspensions, which can contain antioxidants, buffers, bacteriostats and solutes. Other components that can be present in such pharmaceutical compositions include water, alcohols, polyols, glycerine and vegetable oils, for example. Compositions adapted for parenteral administration can be presented in unit-dose or multi-dose containers (e.g., sealed ampoules and vials), and can be stored in a freeze-dried (i.e., lyophilized) condition requiring the addition of a sterile liquid carrier (e.g., sterile saline solution for injections) immediately prior to use. Extemporaneous injection solutions and suspensions can be prepared from sterile powders, granules and tablets.

Pharmaceutical compositions adapted for transdermal administration can be provided as discrete patches intended to remain in intimate contact with the epidermis for a prolonged period of time. Pharmaceutical compositions adapted for topical administration can be provided as, for example, ointments, creams, suspensions, lotions, powders, solutions, pastes, gels, sprays, aerosols or oils. A topical ointment or cream is preferably used for topical administration to the skin, mouth, eye or other external tissues. When formulated in an ointment, the active ingredient can be employed with either a paraffinic or a water-miscible ointment base. Alternatively, the active ingredient can be formulated in a cream with an oil-in-water base or a water-in-oil base.

Pharmaceutical compositions adapted for topical administration to the eye include, for example, eye drops or injectable pharmaceutical compositions. In these pharmaceutical compositions, the active ingredient can be dissolved or suspended in a suitable carrier, which includes, for example, an aqueous solvent with or without carboxymethylcellulose. Pharmaceutical compositions adapted for topical administration in the mouth include, for example, lozenges, pastilles and mouthwashes.

Pharmaceutical compositions adapted for nasal administration can comprise solid carriers, such as, powders (preferably having a particle size in the range of 20 to 500 microns). Powders can be administered in the manner in which snuff is taken, i.e., by rapid inhalation through the nose from a container of powder held close to the nose. Alternatively, pharmaceutical compositions adopted for nasal administration can comprise liquid carriers, such as, for example, nasal sprays or nasal drops. These pharmaceutical compositions can comprise aqueous or oil solutions of an autophagy inducing or autophagy inducing compound. Compositions for administration by inhalation can be supplied in specially adapted devices including, but not limited to, pressurized aerosols, nebulizers or insufflators, which can be constructed so as to provide predetermined dosages of the autophagy inducing or autophagy inducing compound.

Typically, pharmaceutical compositions for injection or intravenous administration are solutions in sterile aqueous buffers. Where necessary, the composition can also include a solubilizing agent and a local anesthetic, such as, lidocaine to ease pain at the site of the injection. Generally, the ingredients are supplied either separately or mixed together in unit dosage form, for example, as a dry lyophilized powder or water-free concentrate in a hermetically sealed container, such as, an ampoule or sachet indicating the quantity of active agent.

Where the composition is to be administered by infusion, it can be dispensed with an infusion bottle, bag, or other acceptable container, containing sterile pharmaceutical grade water, saline, or other acceptable diluents. Where the composition is administered by injection, an ampoule of sterile water for injection or saline can be provided so that the ingredients may be mixed prior to administration.

For a patient who cannot orally ingest a nutrient, it is essential to supply all nutrients, such as, an amino acid, a saccharide and an electrolyte through a vein. This way is called the total parenteral nutrition therapy, (TPN therapy) which can be provided by a TPN solution. Such TPN solutions are particularly suitable for critically ill patients for a therapy in the Intensive Care Unit. As a TPN solution employed in the TPN therapy, there has been known (1) a TPN solution containing a saccharide, an amino acid, a fat and an electrolyte (Japanese Unexamined Patent Publications No. 186822/1989, WO8503002 and EP-A-0 399 341), (2) an emulsion for injection comprising an amino acid and a fat (Japanese Unexamined Patent Publication No. 74637/1986), (3) a TPN solution comprising two separate infusions, one of which contains glucose and an electrolyte and the other of which contains an amino acid (Japanese Unexamined Patent Publications No. 52455/1982 and No. 103823/1986) and the like. In the TPN therapy, an infusion containing a high concentration of saccharide is usually administered to a patient.

As indicated the high nutritional content of such TPN solutions may lead to hyperglycemia and has been found to have a detrimental effect on the repair processes in critically ill patients by inhibition the autophagy process, which contributes to the removal of damaged organelles.

Accordingly, a further aspect of the invention relates to a TPN solution combined with an autophagy inducing or autophagy inducing compound of the invention. This combined composition is used to improve the condition of a critically ill patient or to reduce or treat multiple organ dysfunction syndrome in a critically ill patient.

Compositions for parenteral nutrition, in particular, for intravenous administration are isotonic or hypertonic solutions (e.g., prepared by NaCl and/or dextrose or lactated Ringers) further comprising a saccharide, such as, glucose in a range between 10% and 20% (w/v) to obtain a high nutritional content, and further comprising lipids and/or amino acids and/or added vitamins.

Compositions for parenteral administration comprise further to autophagy inducing or autophagy inducing compound of the invention a saccharide, such as, glucose. Final glucose concentrations in a composition for administration are typically in the range from 10% to 20% (w/v) e.g., 12.5%, or 16%.

Compositions for parenteral administration typically further comprise saturated, mono-unsaturated and essential poly-unsaturated fatty acids, such as, refined olive oil and/or soybean oil. Final lipid concentrations in a composition for administration are typically in the range of 2% to 6% (w/v) e.g., 4%.

Compositions for parenteral administration typically further comprise one or more amino acids. Final amino acid concentrations are typically in the range from 2% to 6% (w/v) e.g., 4%.

Compositions for parenteral administration optionally further comprise trace elements, such as, one or more of Fe, Zn, Cu, Mn, F, Co, I, Se, Mo, Cr e.g., under the form of, respectively, the following salts ferrous gluconate, copper gluconate, manganese gluconate, zinc gluconate, sodium fluoride, cobalt II gluconate, sodium iodide, sodium selenite, ammonium molybdate and chromic chloride.

Compositions for parenteral administration optionally further comprise one or more vitamins, such as, Vitamin A (Retinol), Vitamin D3, Vitamin E (a tocopherol), Vitamin C, Vitamin B1 (thiamine), Vitamin B2 (riboflavin), Vitamin B6 (pyridoxine), Vitamin B12, Folic Acid, Pantothenic acid, Biotin, and Vitamin PP (niacin), e.g., under the form of Retinol palmitate, Colecalciferol, DL-α-tocopherol, Ascorbic acid, Cocarboxylase tetrahydrate, Riboflavin dihydrated sodium phosphate, Pyridoxine hydrochloride, Cyanocobalamin, Folic acid, Dexpanthenol, D-Biotin and Nicotinamide.

Compositions for parenteral administration prior to administration can be isotonic solutions, or more particularly hypertonic solutions e.g., solutions with osmolarity between 1000 and 1500, or between 1200 and 1500 mOsm/liter, e.g., 1250 or 1500 mOsm/liter.

Compositions for parenteral administration can be provided as one solution comprising all constituents or as a kit of parts wherein different constituents are provided separately (saccharide, lipids, amino acids) and wherein the autophagy inducing or autophagy inducing compound is dissolved in one of the constituents or is provided separately. One or more of the different constituents may be provided in a dried form, which is redissolved prior to use.

The compositions for parenteral nutrition in accordance with the invention further comprise a autophagy inducing or autophagy inducing compound, such as, spermidine or spermine, autophagy inducing or autophagy inducing compound or putrescine in a concentration between 0.05%, 0.1%, 0.2% or 0.5% to 1%, 2%, 3% or 4% (w/v).

The compositions for intravenous administration are typically packed in plastic bags with spike ports for delivery by intravenous drips.

In a specific embodiment, the present compositions contain spermine or autophagy inducing or autophagy inducing compound.

For patients who do not rely on parenteral food pharmaceutical compositions, herein described, can be provided in the fomi of oral tablets, capsules, elixirs, syrups and the like.

Compositions for oral administration might require an enteric coating to protect the composition(s) from degradation within the gastrointestinal tract. In another example, the composition(s) can be administered in a liposomal formulation to shield the autophagy inducing or autophagy inducing compound, disclosed herein, from degradative enzymes, facilitate the molecule's transport in the circulatory system, and affect delivery of the molecule across cell membranes to intracellular sites.

An autophagy inducing or autophagy inducing compound intended for oral administration can be coated with or admixed with a material (e.g., glyceryl monostearate or glyceryl distearate) that delays disintegration or affects absorption of the autophagy inducing or autophagy inducing compound in the gastrointestinal tract. Thus, for example, the sustained release of an autophagy inducing or autophagy inducing compound can be achieved over many hours and, if necessary, the autophagy inducing or autophagy inducing compound can be protected from being degraded within the gastrointestinal tract. Taking advantage of the various pH and enzymatic conditions along the gastrointestinal tract, pharmaceutical compositions for oral administration can be formulated to facilitate release of an autophagy inducing or autophagy inducing compound at a particular gastrointestinal location.

Selectively permeable membranes surrounding an osmotically active driving compound are also suitable for orally administered compositions. Fluid from the environment surrounding the capsule is imbibed by the driving compound, which swells to displace the autophagy inducing or autophagy inducing compound through an aperture, can provide an essentially zero order delivery profile instead of the spiked profiles of immediate release formulations. A time delay material, such as, but not limited to, glycerol monostearate or glycerol stearate can also be used.

Suitable pharmaceutical carriers also include starch, glucose, lactose, sucrose, gelatin, saline, gum acacia, talc, keratin, urea, malt, rice, flour, chalk, silica gel, sodium stearate, glycerol monostearate, talc, sodium chloride, dried skim milk, glycerol, propylene, glycol, water, and ethanol. If desired, the carrier can also contain minor amounts of wetting or emulsifying agents, or pH buffering agents. In addition, auxiliary, stabilizing, thickening, lubricating, and coloring agents may be used. The composition can be formulated as a suppository with traditional binders and carriers, such as, triglycerides.

For oral administration in the form of a tablet or capsule, the active drug component can be combined with an oral, non-toxic, pharmaceutically acceptable, inert carrier, such as, but not limited to, lactose, starch, sucrose, glucose, methyl cellulose, magnesium stearate, dicalcium phosphate, calcium sulfate, mannitol, and sorbitol. For oral administration in liquid form, the oral drug components can be combined with any oral, non-toxic, pharmaceutically acceptable carrier, such as, but not limited to, ethanol, glycerol, and water. Moreover, suitable binders, lubricants, disintegrating agents and coloring agents can also be incorporated into the mixture. Suitable binders include, but are not limited to, starch, gelatin, natural sugars (e.g., glucose, beta-lactose), corn sweeteners, natural and synthetic gums (e.g., acacia, tragacanth, sodium alginate), carboxymethylcellulose, polyethylene glycol, and waxes. Lubricants useful for an orally administered drug, include, but are not limited to, sodium oleate, sodium stearate, magnesium stearate, sodium benzoate, sodium acetate, and sodium chloride. Disintegrators include, but are not limited to, starch, methyl cellulose, agar, bentonite, and xanthan gum.

Pharmaceutical compositions adapted for oral administration can be provided, for example, as capsules or tablets; as powders or granules; as solutions, syrups or suspensions (in aqueous or non-aqueous liquids); as edible foams or whips; or as emulsions. For oral administration in the fouu of a tablet or capsule, the active drug component can be combined with an oral, non-toxic, pharmaceutically acceptable, inert carrier, such as, but not limited to, lactose, starch, sucrose, glucose, methyl cellulose, magnesium stearate, dicalcium phosphate, magnesium carbonate, stearic acid or salts thereof, calcium sulfate, mannitol, and sorbitol. For oral administration in the form of a soft gelatin capsule, the active drug component can be combined with an oral, non-toxic, pharmaceutically acceptable, inert carrier, such as, but not limited to, vegetable oils, waxes, fats, semi-solid, and liquid polyols. For oral administration in liquid form, the oral drug components can be combined with any oral, non-toxic, pharmaceutically acceptable carrier, such as, but not limited to, ethanol, glycerol, polyols, and water. Moreover, suitable binders, lubricants, disintegrating agents and coloring agents can also be incorporated into the mixture. Suitable binders include, but are not limited to, starch, gelatin, natural sugars (e.g., glucose, beta-lactose), corn sweeteners, natural and synthetic gums (e.g., acacia, tragacanth, sodium alginate), carboxymethylcellulose, polyethylene glycol, and waxes. Lubricants useful for an orally administered drug, include, but are not limited to, sodium oleate, sodium stearate, magnesium stearate, sodium benzoate, sodium acetate, and sodium chloride. Disintegrators include, but are not limited to, starch, methyl cellulose, agar, bentonite, and xanthan gum.

Orally administered compositions may contain one or more agents, for example, sweetening agents, such as, but not limited to, fructose, aspartame and saccharin. Orally administered compositions may also contain flavoring agents, such as, but not limited to, peppermint, oil of wintergreen, and cherry. Orally administered compositions may also contain coloring agents and/or preserving agents.

The autophagy inducing or autophagy inducing compounds of the invention can also be administered in the form of liposome delivery systems, such as, small unilamellar vesicles, large unilamellar vesicles and multilamellar vesicles. Liposomes can be formed from a variety of phospholipids, such as, cholesterol, stearylamine or phosphatidylcholines. A variety of cationic lipids can be used in accordance with the invention including, but not limited to, N-(1(2,3-dioleyloxy)propyl)-N,N,N-trimethylammonium chloride (“DOTMA”) and diolesylphosphotidylethanolamine (“DOPE”). Such compositions suit the mode of administration.

The autophagy inducing or autophagy inducing compounds of the invention can also be delivered by the use of monoclonal antibodies as individual carriers to which the compounds can be coupled. The compounds can also be coupled with soluble polymers as targetable drug carriers. Such polymers can include polyvinylpyrrolidone, pyran copolymer, polyhydroxypropylmethacrylamide-phenol, polyhydroxyethylaspartamide-phenol, or polyethyleneoxide-polylysine substituted with palmitoyl residues. Furthermore, the autophagy inducing or autophagy inducing compounds can be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

Pharmaceutical compositions adapted for rectal administration can be provided as suppositories or enemas. Pharmaceutical compositions adapted for vaginal administration can be provided, for example, as pessaries, tampons, creams, gels, pastes, foams or spray formulations.

Suppositories generally contain active ingredients in the range of 0.5% to 10% by weight. Oral formulations preferably contain 10% to 95% active ingredient by weight. In a preferred embodiment, the composition is formulated in accordance with routine procedures as a pharmaceutical composition adapted for intratumoral injection, implantation, subcutaneous injection, or intravenous administration to humans.

Conveniently, the blood autophagy inducing or autophagy inducing compound level is kept within the ranges mentioned in connection with the invention for as long a period of time as the patient is critically ill. Hence, as a general rule, the blood autophagy inducing or autophagy inducing compound level is kept within the ranges mentioned in connection with the invention as long as the patient is critically ill. Consequently, the blood autophagy inducing or autophagy inducing compound level is usually kept within the ranges mentioned in connection with the invention for a period of time of more than about 8 hours, preferably more than about 24 hours, even more preferred more than about 2 days, especially more than about 4 days, and even more than about 7 days. In certain cases, it may even be preferred that the blood autophagy inducing or autophagy inducing compound level is kept within the ranges mentioned in connection with the invention after the patient (previously) considered as being critically ill has been transferred from the Intensive Care Unit to another part of the hospital or even after the patient has left the hospital.

A critically ill patient, optionally entering an ICU, may be fed continuously, on admission with mainly intravenous glucose (for example, about 200 g to about 300 g per 24 hours) and from the next day onward with a standardized feeding schedule aiming for a caloric content up to between about 10 and about 40, preferably between about 20 and about 30, non-protein calories/kg/24 hours and a balanced composition (for example, between about 0.05 and about 0.4, preferably between about 0.13 and about 0.26, g nitrogen/kg/24 hours and between about 20% and about 40% of non-protein calories as lipids) of either total parenteral, combined parenteral/enteral or full enteral feeding, the latter mode attempted as early as possible. Other concomitant ICU therapy can be left to the discretion of attending physicians.

Alternatively, the following procedure can be used or it is possible to use a combination or variant of these procedures, as the physician considers advantageous for the patient:

A critically ill patient may be fed, on the admission day, using, for example, a 20% glucose infusion and from day 2 onward by using a standardized feeding schedule consisting of normal caloric intake (for example, about 25-35 calories/kgBW/24 h) and balanced composition (for example, about 20%-40% of the non-protein calories as lipids and about 1-2 g/kgBW/24 h protein and about 0.01-100 mg/kg BW/24 h autophagy inducing or autophagy inducing compound) of either total parenteral, combined parenteral/enteral or full enteral feeding, the route of administration of feeding depending on assessment of feasibility of early enteral feeding by the attending physician. All other treatments, including feeding regimens, were according to standing orders currently applied within the ICU.

The autophagy inducing or autophagy inducing compound and optionally another therapeutic agent are administered at an effective dose. The dosing and regimen most appropriate for patient treatment will vary with the disease or condition to be treated, and in accordance with the patient's weight and with other parameters.

An effective dosage and treatment protocol can be determined by conventional means, comprising the steps of starting with a low dose in laboratory animals, increasing the dosage while monitoring the effects (e.g., histology, disease activity scores), and systematically varying the dosage regimen. Several factors may be taken into consideration by a clinician when determining an optimal dosage for a given patient. Additional factors include, but are not limited to, the size of the patient, the age of the patient, the general condition of the patient, the particular disease being treated, the severity of the disease, the presence of other drugs in the patient, and the in vivo activity of the autophagy inducing or autophagy inducing compound.

A typical effective human dose of an autophagy inducing or autophagy inducing compound would be from about 1 μg/kg body weight/day to about 100 mg/kg/day, preferably, from about 5 μg/kg/day to about 50 mg/kg/day, and most preferably about 10 μg/kg/day to 20 mg/kg/day. As analogues of the autophagy inducing or autophagy inducing compound, disclosed herein, can be 2 to 100 times more potent than naturally occurring counterparts, a typical effective dose of such an analog can be lower, for example, from about 10 ng/kg body weight/day to 1 mg/kg/day, preferably, 1 μg/kg/day to 900 μg/kg/day, and even more preferably 2 μg/kg/day to 250 μg/kg/day.

In another embodiment, the effective dose of an autophagy inducing or autophagy inducing compound of the present is less than 1 μg/kg/day. In yet another embodiment, the effective dose of an autophagy inducing or autophagy inducing compound of the present is greater than 100 mg/kg/day.

The specific dosage for a particular patient, of course, has to be adjusted to the degree of response, the route of administration, the patient's weight, and the patient's general condition, and is finally dependent upon the judgment of the treating physician. Especially the highly critical condition of ICU patients requires a specific dosage and dosage regime.

It is understandable that the ideal dosage per serving to have the health effect will have to vary according the body weight of the subject who consumes the oral ingestible dosage form which comprises the autophagy inducing or autophagy inducing compound of the invention. A beneficial effect can be obtained in a subject with about 50 kg body weight by an orally ingestible dosage form comprising between 0.05 mg and 5 grams, preferably, 0.25 mg to 2 grams, more preferably between 0.5 mg and 1.5 grams, more preferably between 1 mg and 750 mg of the autophagy inducing or autophagy inducing compound of the invention per administration (as demonstrated in Table 1).

TABLE 1
Possible amount of the autophagy inducing or autophagy inducing compound active
ingredient of the invention per serving by a subject (BW: body weight).

| | BW/kg | | | | | | | | | |
|---|---|---|---|---|---|---|---|---|---|---|
| Dose | 50 | 60 | 70 | 80 | 90 | 100 | 110 | 120 | 130 | 140 |
| mg/kg | mg | mg | mg | mg | mg | mg | mg | mg | mg | Mg |
| 0.1 | 5 | 6 | 7 | 8 | 9 | 10 | 11 | 12 | 13 | 14 |
| 0.2 | 10 | 12 | 14 | 16 | 18 | 20 | 22 | 24 | 26 | 28 |
| 0.3 | 15 | 18 | 21 | 24 | 27 | 30 | 33 | 36 | 39 | 42 |
| 0.4 | 20 | 24 | 28 | 32 | 36 | 40 | 44 | 48 | 52 | 56 |
| 0.5 | 25 | 30 | 35 | 40 | 45 | 50 | 55 | 60 | 65 | 70 |
| 1 | 50 | 60 | 70 | 80 | 90 | 100 | 110 | 120 | 130 | 140 |
| 5 | 250 | 300 | 350 | 400 | 450 | 500 | 550 | 600 | 650 | 700 |
| 10 | 500 | 600 | 700 | 800 | 900 | 1000 | 1100 | 1200 | 1300 | 1400 |
| 15 | 750 | 900 | 1050 | 1200 | 1350 | 1500 | 1650 | 1800 | 1950 | 2100 |
| 20 | 1000 | 1200 | 1400 | 1600 | 1800 | 2000 | 2200 | 2400 | 2600 | 2800 |
| 25 | 1250 | 1500 | 1750 | 2000 | 2250 | 2500 | 2750 | 3000 | 3250 | 3500 |
| 30 | 1500 | 1800 | 2100 | 2400 | 2700 | 3000 | 3300 | 3600 | 3900 | 4200 |
| 35 | 1750 | 2100 | 2450 | 2800 | 3150 | 3500 | 3850 | 4200 | 4550 | 4900 |
| 40 | 2000 | 2400 | 2800 | 3200 | 3600 | 4000 | 4400 | 4800 | 5200 | 5600 |
| 45 | 2250 | 2700 | 3150 | 3600 | 4050 | 4500 | 4950 | 5400 | 5850 | 6300 |
| 50 | 2500 | 3000 | 3500 | 4000 | 4500 | 5000 | 5500 | 6000 | 6500 | 7000 |

A beneficial effect can also be obtained in a subject with about 50 kg body weight as part of a TPN therapy comprising between 0.05 mg and 2.5 grams, preferably 0.5 mg to 2 grams, more preferably between 1 mg and 1.5 grams, more preferably between 2 mg and 750 mg of the autophagy inducing or autophagy inducing compound of the invention per administration.

Another aspect concerns treatment by an authophagy activator or enhancer a disorder of bone metabolism in a subject, for instance, to reverse a disorder of decrease in bone formation and an increase in bone resorption. Such disorder can be caused by critical illness. Particular aspects of the embodiments are the following. An autophagy activator or autophagy inducing compound for use as a medicine to inhibit increased bone resorption. An autophagy activator or autophagy inducing compound for use as a medicine to inhibit osteoclast activity. An autophagy activator or autophagy inducing compound for use as a medicine to inhibit the formation of osteoclast (TRAP positive multinuclear cells) from blood progenitor cells. An autophagy activator or autophagy inducing compound for use as a medicine to activate bone formation. An autophagy activator or autophagy inducing compound for use as a medicine to treat a bone degenerative disorder, which is a bone metabolism disorder. An autophagy activator or autophagy inducing compound for use as a medicine to treat a bone degenerative disorder, which is caused by acute critical illness or prolonged critical illness. An autophagy activator or autophagy inducing compound for use as a medicine to treat a bone degenerative disorder, which is caused by (parenteral) nutrition induced suppression of autophagy. An autophagy activator or autophagy inducing compound for use as a medicine to treat a bone degenerative disorder, which is caused by mTOR activation. And/or an autophagy activator or autophagy inducing compound for use as a medicine to treat a bone degenerative disorder, which is hyperresorption of bone.

An embodiment concerns an autophagy activator or autophagy inducing compound for use as a medicine to treat a bone degenerative disorder that is pathological increased bone resorption and pathological decreased bone formation. An autophagy activator or autophagy inducing compound for use as a medicine to treat a bone degenerative disorder that is a disrupted calcium and bone metabolism by acute critical illness and/or prolonged critical illness. Another embodiment concerns an autophagy activator or autophagy inducing compound for use as a medicine to treat a bone degenerative disorder that is pathological increased bone resorption and pathological decreased bone formation. An autophagy activator or autophagy inducing compound for use as a medicine to treat a bone degenerative disorder caused by enhanced osteoclast formation from circular precursors in the blood during critical illness. Yet an embodiment concerns an autophagy activator or autophagy inducing compound for use as a medicine to treat a bone degenerative disorder that is pathological increased bone resorption and pathological decreased bone formation. An autophagy activator or autophagy inducing compound for use as a medicine to treat a bone degenerative disorder that is increased osteoclast formation from circulating precursor leading to osteoporosis.

There are autophagy-inducing compounds for the above embodiments of the invention. For instance, the macrolide autophagy inducing compounds. The macrolides are a group of drugs (typically antibiotics) whose activity stems from the presence of a macrolide ring, a large macrocyclic lactone ring to which one or more deoxy sugars, usually cladinose and desosamine, may be attached. The lactone rings are usually 14-, 15-, or 16-membered. Macrolides belong to the polyketide class of natural products. There are several macrolides authophagy inducers, for instance, the Azithromycin macrolide (an azalide, a subclass of macrolide antibiotics, IUPAC name: 2R,3S,4R,5R,8R,10R,11R,12S,13S,14R)-2-ethyl-3,4,10-trihydroxy-3,5,6,8,10,12,14-heptamethyl-15-oxo-11-{[3,4,6-trideoxy-3-(dimethyl amino)-β-D-xylo-]oxy}-1-oxa-6-azacyclopentadec-13-yl2,6-dideoxy-3-C-methyl-3-O-methyl-α-L-ribo-hexopyranoside) is an autophagy-inducing compound in a dose-dependent manner in a reversible manner and this effect is removable by an autophagy inhibitor, such as, 3-methyladenine or LY294002 (Stamatiou R et al. EUROPEAN RESPIRATORY JOURNAL, Volume: 34 Issue: 3 Pages: 721-730 Published: SEP) 2009).

Other suitable autophagy inducing compounds that are available for use in a medicament, as described above, are the calpain inhibitors (calpain 1 or calpain 2 inhibitors), for instance, of the group consisting of calpastatin, ALLM, calpeptin, leupeptin, α-dicarbonyls, quinolinecarboxamides, sulfonium methyl ketones, diazomethyl ketones, Leu-Abu-CONHEt (AK275), 27-mer calpastatin peptide, Cbz-Val-Phe-H (MDL28170), calpeptin (Z-Leu-Nle-H), α-mercaptoacrylic acids, phosphorus derivatives, epoxysuccinates, acyloxymethyl ketones, halomethylketones and E64 have been demonstrated to induce autophagy (WO2007003941) and are suitable in the manufacture of a medicament for increasing autophagy in an individual. IMPase inhibitors, for instance, of the group consisting of L-690330, lithium, valproate, carbemazapine and salts, analogues and derivatives, thereof, have been demonstrated to induce autophagy (WO2006079792) and are suitable in the manufacture of a medicament for increasing autophagy in an individual. Compounds or agents that inhibit or reduce the activity of the cAMP/EPAC/PLC, for instance, of the group consisting of clonidine, rilmenidine, tyramine, morphine, baclofen, mastoparan, propranolol, bupivacain, N-dodecyl lysinamide, Gsα and/or PACAP are suramin, NF449, NF503 minoxidil, pinacidil, cromakalim or an analog or derivative, thereof, and have been demonstrated to induce autophagy (WO2008099175) and are suitable in the manufacture of a medicament for increasing autophagy in an individual. A dose of glutamine (Gln) such that it induces low millimolar concentrations (for instance, 1-10 mM, preferably 2-4 mM) of ammonium (Gln-derived ammonia) after Gln deamination in mitochondria stimulates autophagy. Christina H. Eng et al.; Autophagy 6:7, 968-970; Oct. 1, 2010; © 2010 Landes Bioscience. This can, for instance, be reached by feeding a patient parenterally with a parenteral nutrition comprising water having dissolved therein from about 1 to 150 mMoles/l of glutamine Trehalose (α,α-Trehalose; α-D-glucopyranosyl-(1→1)-α-D-glucopyranoside with Molar mass 342.296 g/mol (anhydrous) and 378.33 g/mol (dihydrate)), for instance, D-(+)-trehalose dehydrate is an autophagy inducing agent (Casarejos M J et al. Conference Information: 14th International Congress of Parkinsons Disease and Movement Disorders Buenos Aires, ARGENTINA, JUN 13-17, 2010 Casarejos yr:2010 vol:25 iss:7 pg:S417-S417; Catarina Gomes et al. Mutant superoxide dismutase 1 overexpression in NSC-34 cells: Effect of trehalose on aggregation, TDP-43 localization and levels of co-expressed glycoproteins Neuroscience Letters 475 (2010) 145-149 and Jos). These effects can be reached in vivo (e A. Rodríguez-Navarro et al. Neurobiology of Disease 39 (2010) 423-438). For the use the disclosure as described hereing, the dose is such to reach 1 to 100 mM, preferably 2-50 mM or more preferably 5 to 20 mM at the cells. This can, for instance, be reached by delivery trehalose in a parenteral delivery form and replacing a fraction of the carbohydrates so that 0.1-50 mg/kg body weight/day, preferably 0.2-25 mg/kg body weight/day, yet more preferably 0.4-5 mg/kg body weight/day, yet more preferably 0.5-1.5 mg/kg body weight/day and most preferably 1 mg/kg body weight/day is provided. For oral delivery daily dose of Trehalose can be higher, for instance, about 0.5 to about 100 g/adult/day or 0.5 to 100 g/adult/day, and preferably about 1 to about 50 g/adult/day with respect to the amount of Trehalose or its derivates.

Compounds for medical use, as described in the above embodiments, are, for instance, the autophagy inducers Silibinin or Curcumin.

Compounds for medical use, as described herein, can also be one or more compounds of the mTOR-dependent autophagy inducers are compounds, such as, Rapamycin and analogs (CCI-779, RAD001, AP23573), Perhexyline, Amiodaronel, Niclosamide, Rottlerin, Tori-1 (with structure) PI103 and structurally related compounds, Phenethyl isothiocyanate (PEITC) or Dexamethasone

Suitable Rapamycin analogues for use herein are of the group of the Rapamycin derivatives, such as, Everolimus, Temsirolimus, 40-O-(2-hydroxyethyl)-rapamycin, and/or 32-deoxorapamycin, and/or 16-pent-2-ynyloxy-32-deoxorapamycin, and/or 16-pent-2-ynyloxy-32 (S or R)-dihydro-rapamycin, and/or 16-pent-2-ynyloxy-32 (S or R)-dihydro-40-O-(2-hydroxyethyl)-rapamycin, and/or 40-[3-hydroxy-2-(hydroxy-methyl)-2-methylpropanoatel-rapamycin (also known as CCI779) and/or]40-epi-(tetrazolyl)-rapamycin (also known as ABT578), and/or the so-called rapalogs, e.g., as disclosed in WO9802441, WO114387 and WO364383, AP23573, AP23464; AP23675 or AP23841, e.g., AP23573, and/or compounds disclosed under the name TAFA-93, and/or compounds disclosed under the name biolimus. Another preferred rapamycin derivative is selected from the group consisting of 40-O-(2-hydroxyethyl)-rapamycin (also known as everolimus), and/or 32-deoxorapamycin, and/or 16-pent-2-ynyloxy-32-deoxorapamycin, and/or 16-pent-2-ynyloxy-32 (S or R)-dihydro-rapamycin, and/or 16-pent-2-ynyloxy-32 (S or R)-dihydro-40-O-(2-hydroxyethyl)-rapamycin, and/or 40-[3-hydroxy-2-(hydroxy-methyl)-2-methylpropanoate]-rapamycin (also known as CC1779 or temsirolimus) and/or 40-epi-(tetrazolyl)-rapamycin (also known as ABT578), and/or AP23573, such as, 40-O-(2-hydroxyethyl)-rapamycin.

Suitable phenothiazine derivatives for invention are, for example, compounds chosen from quinacrine, imipramine, carbamazepine, phenazine, phenothiazine, promazine, chloropromazine, haloperidol, clozapine, 2-chlorophenothiazine, promethazine (10-(2-dimethylaminopropyl)phenothiazine), chloroprothixen, and acepromazine.

Compounds for medical use, as described in the above embodiments, can also be one or more compounds of the mTOR-independent autophagy inducers are compounds, such as, Lithium, L-690,330, Carbamazepine, sodium valproate, Verapamil, loperamide, Amiodarone, nimodipine, Nitrendipine, niguldipine, Nicardipine, pimozide, Calpastatin, calpeptin, Clonidine, rilmenidine, 2′,5′-Dideoxyadenosine, NF449, Minoxidil, Penitrem A, Trehalose, Spermidine, Resveratrol, Fluspirilene, trifluoperazine, SMER10, SMER18, SMER28 OR Compound C (dorsomorphin).

A method of inhibiting critical illness [MeSH Descriptor: C23.550.291.625] enhanced osteoclastogenesis in a patient in need thereof, comprising administering to the patient an autophagy activating compound, wherein the patient suffers from a condition selected from the group consisting of Critical Illness Bone Atrophy, Metabolic Bone Disease Following ICU Admission, Critical Illness Related Metabolic Bone Disease and Osteoporosis Secondary to ICU Admission and whereby the autophagy activating compound is administered in an amount sufficient to critical illness-induced osteoclastogenesis in the patient. One embodiment provides autophagy inducing compounds administered in a dose such that plasma levels are reached sufficient to prevent or inhibit PBMCs in critically ill patients or differentiation into osteoclasts. The autophagy inducing compound can be any one of the group of glutamine, resveratrol, fenofibrate, Rapamycin, Spermidine, Everolimus, Promethazine or a derivative thereof or any of the autophagy inducing compounds of the invention, for instance, disclosed in this application.

An autophagy inducing compound of the invention for use in a method of inhibiting critical illness [MeSH Descriptor: C23.550.291.625] enhanced osteoclastogenesis in a patient in need thereof, comprising administering to the patient the autophagy activating compound such that plasma levels reached sufficient prevent or inhibit PBMCs in critically ill patients of differentiating into osteoclasts, wherein the patient suffers from a condition selected from the group consisting of Critical Illness Bone Atrophy, Metabolic Bone Disease Following ICU Admission, Critical Illness Related Metabolic Bone Disease and Osteoporosis Secondary to ICU Admission and whereby the autophagy activating compound is administered in an amount sufficient to critical illness-induced osteoclastogenesis in the patient. The invention provides in an embodiment that autophagy inducing compounds are administered in a dose such that plasma levels are reached sufficient prevent or inhibit PBMCs in critically ill patients of differentiating into osteoclasts. The autophagy inducing compound can be any one of the group of glutamine, resveratrol, fenofibrate, Rapamycin, Spermidine, Everolimus, Promethazine or a derivative thereof or any of the autophagy inducing compounds of the invention, for instance, disclosed in this application.

Some of the other embodiments hereof are

### Embodiment 1

An autophagy activator compound for use as a medicine to cure or prevent bone degenerative disorder in a mammalian subject wherein the autophagy activator compound is glutamine and wherein the bone degenerative disorder is caused by acute critical illness or prolonged critical illness.

### Embodiment 2

The autophagy activator compound according to embodiment 1, wherein the bone degenerative disorders is osteoclast activity increased due to acute critical illness and/or prolonged critical illness.

### Embodiment 3

The autophagy activator compound according to any one of the previous embodiments, wherein the bone degenerative disorders is increased osteoclast formation due to autophagy deficiency disorder of critically ill patients.

### Embodiment 4

The autophagy activator compound according to any one of the previous embodiments, wherein the bone degenerative disorders is caused by enhanced osteoclast formation from circulating precursors in the blood during critical illness.

### Embodiment 5

The autophagy activator compound, according to any one of the previous embodiments, wherein the bone degenerative disorder is enhanced osteoclast formation from circulating precursors due to autophagy deficiency disorder.

### Embodiment 6

An autophagy activator compound for use as a medicine to cure or prevent bone degenerative disorder in a mammalian subject wherein the autophagy activator compound is glutamine and wherein the bone degenerative disorder is caused by (parenteral) nutrition-induced suppression of autophagy.

### Embodiment 7

The autophagy activator compound according to any one of the previous embodiments, wherein the bone degenerative disorders is increased osteoclast formation due to autophagy deficiency disorder by (parenteral) nutrition-induced suppression of autophagy in critically ill patients.

### Embodiment 8

The autophagy activator compound according to any one of the previous embodiments, wherein the bone degenerative disorders are caused by enhanced osteoclast formation from circulating precursors in the blood during critical illness (parenteral) nutrition-induced suppression of autophagy.

### Embodiment 9

The autophagy activator compound according to any one of embodiments 6 to 8, wherein the bone degenerative disorder is enhanced osteoclast formation from circulating precursors due to autophagy deficiency disorder.

### Embodiment 10

The autophagy activator compound according to any one of the previous embodiments, wherein the autophagy activator compound is glutamine.

### Embodiment 11

The autophagy activator compound, according to any one of embodiments 1 to 10, wherein the glutamine compound is administered to the patient or mammalian subject such that it induces low millimolar concentrations (for instance, 1-10 mM, preferably 2-4 mM) of ammonium (glutamine-derived ammonia) after glutamine deamination in mitochondria that stimulates autophagy.

### Embodiment 12

The autophagy activator compound, according to any one of the previous embodiments 1 to 10, whereby the compound is administered to the patient or mammalian subject by delivering the compound in a watery fluid delivery (for instance, enteral or parenteral) comprising the watery fluid having dissolved therein from 1 to 150 mMoles/1 of glutamine.

Some of the other embodiments hereof are set forth directly below:

### Embodiment 1

An autophagy activator compound for use as a medicine to cure or prevent bone degenerative disorder in a mammalian subject wherein the autophagy activator compound is a trehalose, for instance, selected from the group consisting of α-D-glucopyranosyl-(1→1)-α-D-glucopyranoside, α-D-glucopyranosyl-(1→1)-β-D-glucopyranoside and β-D-glucopyranosyl-(1→1)-β-D-glucopyranoside or a trehalose-based polymer, such as, poly(6-Vinyladipoyl-Trehalose) and wherein the bone degenerative disorder is caused by acute critical illness or prolonged critical illness.

### Embodiment 2

The autophagy activator compound of embodiment 1, wherein the bone degenerative disorders is osteoclast activity increased due to acute critical illness and/or prolonged critical illness.

### Embodiment 3

The autophagy activator compound of any one of the previous embodiments, wherein the bone degenerative disorder is increased osteoclast formation due to autophagy deficiency disorder of critically ill patients.

### Embodiment 4

The autophagy activator compound, of any one of the previous embodiments, wherein the bone degenerative disorders are caused by enhanced osteoclast formation from circulating precursors in the blood during critical illness.

### Embodiment 5

The autophagy activator compound, according to any one of the previous embodiments, wherein the bone degenerative disorder is enhanced osteoclast formation from circulating precursors due to autophagy deficiency disorder.

### Embodiment 6

An autophagy activator compound for use as a medicine to cure or prevent bone degenerative disorder in a mammalian subject wherein the autophagy activator compound is a trehalose, for instance, selected from the group consisting of α-D-glucopyranosyl-(1→1)-α-D-glucopyranoside, α-D-glucopyranosyl-(1→1)-β-D-glucopyranoside and β-D-glucopyranosyl-(1→1)-β-D-glucopyranoside or a trehalose-based polymer, such as, poly(6-Vinyladipoyl-Trehalose) and wherein the bone degenerative disorder is caused by (parenteral) nutrition-induced suppression of autophagy.

### Embodiment 7

The autophagy activator compound, according to any one of the previous embodiments, wherein the bone degenerative disorder is increased osteoclast formation due to autophagy deficiency disorder caused by (parenteral) nutrition-induced suppression of autophagy in critically ill patients.

### Embodiment 8

The autophagy activator compound, according to any one of the previous embodiments, wherein the bone degenerative disorder is caused by enhanced osteoclast formation from circulating precursors in the blood during critical illness (parenteral) nutrition-induced suppression of autophagy.

### Embodiment 9

The autophagy activator compound, according to any one of the previous embodiments 6 to 8, wherein the bone degenerative disorder is enhanced osteoclast formation from circulating precursors due to autophagy deficiency disorder.

### Embodiment 10

The autophagy activator compound, according to any one of the previous embodiments, wherein the autophagy activator compound is a trehalose, for instance, selected from the group consisting of α-D-glucopyranosyl-(1→1)-α-D-glucopyranoside, α-D-glucopyranosyl-(1→1)-β-D-glucopyranoside and β-D-glucopyranosyl-(1→1)-D-glucopyranoside or a trehalose-based polymer, such as, poly(6-Vinyladipoyl-Trehalose) inhibits osteoclast activity.

### Embodiment 11

The autophagy activator compound, according to any one of the previous embodiments, wherein the compound is administered to the patient or mammalian subject such to reach 1 to 100 mM, preferably 2-50 mM or more preferably 5 to 20 mM at the cells.

### Embodiment 12

The autophagy activator compound, according to any one of embodiments 1 to 10, wherein the compound is administered to the patient or mammalian subject by delivering the compound in a parenteral delivery form and replacing a fraction of the carbohydrates of having a concentration so that 0.1-50 mg/kg body weight/day, preferably 0.2-25 mg/kg body weight/day, yet more preferably 0.4-5 mg/kg body weight/day, yet more preferably 0.5-1.5 mg/kg body weight/day and most preferably 1 mg/kg body weight/day of the compound is provided.

### Embodiment 13

The autophagy activator compound, according to any one of embodiments 1 to 10, wherein the compound is administered to the patient or mammalian subject by delivering the compound in a parenteral delivery form an enteral dose of about 0.5 to about 100 g/adult/day or 0.5 to 100 g/adult/day, and preferably about 1 to about 50 g/adult/day with respect to the amount of compound is provided.

### Embodiment 14

The autophagy activator compound, according to any one of embodiments 1 to 13, in combination with glutamine.

### Embodiment 15

The autophagy activator compound, according to any one of embodiments 1 to 13, in combination with a glutamine compound wherein the glutamine compound is a dose such that it induces low millimolar concentrations (for instance, 1-10 mM, preferably 2-4 mM) of ammonium (glutamine-derived ammonia) after glutamine deamination in mitochondria, which stimulates autophagy when administered to the patient or mammalian subject.

### Embodiment 16

The autophagy activator compound, according to any one of embodiments 1 to 13, in combination with glutamine wherein the compounds are in a watery fluid delivery form comprising the watery fluid having dissolved therein from 1 to 150 mMoles/1 of glutamine for enteral or parenteral administration to the patient or mammalian subject.

Some of the other embodiments hereof are set forth directly below:

### Embodiment 1

An autophagy activator compound for use as a medicine to cure or prevent bone degenerative disorder caused by acute critical illness or prolonged critical illness in a mammalian subject.

### Embodiment 2

An autophagy activator compound for use as a medicine to inhibit osteoclast activity increase due to autophagy deficiency disorder caused by parenteral or enteral nutrition-induced suppression of autophagy.

### Embodiment 3

An autophagy activator compound for use as a medicine according to any one of embodiments 1 to 2, wherein the bone degenerative disorder is increased osteoclast activity.

### Embodiment 4

Embodiment 1, 2 or 3, wherein the autophagy activator compound is selected from the group consisting of: (a) Loperamide

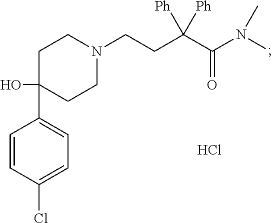

### (c) Niguldipine

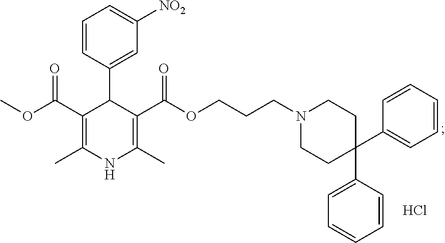 [SMILES: COC(=O)C1=C(C)NC(C)=C(C(=O)OCCCN2CCC(c3ccccc3)(c3ccccc3)CC2)C1c1cccc([N+](=O)[O-])c1 ; Cl]

### (d) Pimozide

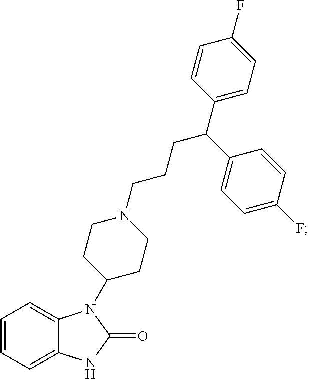 [SMILES: O=c1[nH]c2ccccc2n1C1CCN(CCCC(c2ccc(F)cc2)c2ccc(F)cc2)CC1]

### (e) Nicardipine

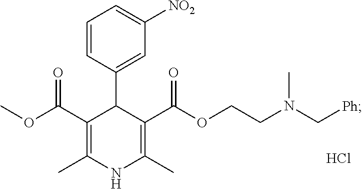 [SMILES: COC(=O)C1=C(C)NC(C)=C(C(=O)OCCN(C)Cc2ccccc2)C1c1cccc([N+](=O)[O-])c1 ; Cl]

### (f) Penitrem A

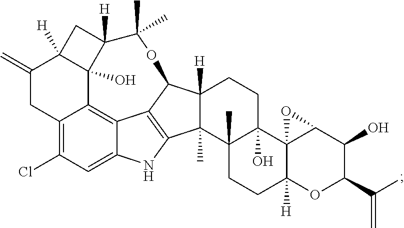

### (g) Fluspirilene

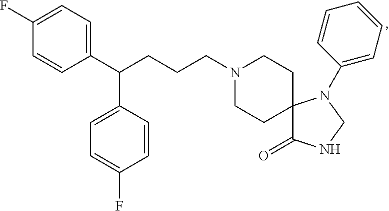 [SMILES: O=C1NCN(c2ccccc2)C12CCN(CCCC(c1ccc(F)cc1)c1ccc(F)cc1)CC2]

### (h) Trifluoperazine

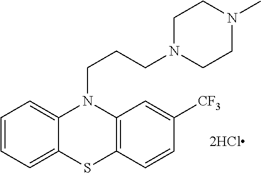 [SMILES: CN1CCN(CCCN2c3ccccc3Sc3ccc(C(F)(F)F)cc32)CC1]

### Embodiment 5

Embodiment 1, 2 or 3, whereby the autophagy activator compound is selected from the group consisting of: (a) 10-NCP

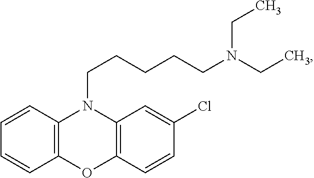 [SMILES: CCN(CC)CCCCCN1c2ccccc2Oc2ccc(Cl)cc21]

### (b) Promazine

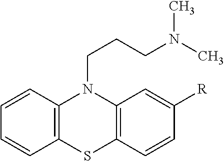

whereby R=H, (c) Chloropromazine whereby R=Cl

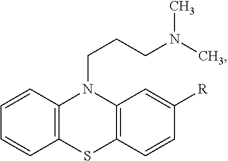

### (d) Triflupromazine

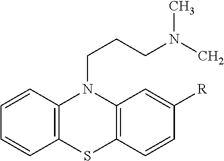

whereby R=CF₃, (e) Prometazine

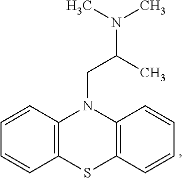 [SMILES: CC(CN1c2ccccc2Sc2ccccc21)N(C)C]

### (f) Mesoridazine

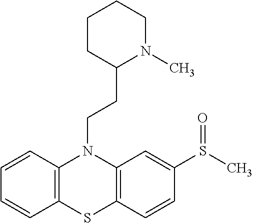 [SMILES: CN1CCCCC1CCN1c2ccccc2Sc2ccc(S(C)=O)cc21]

### and (g) Thioridazine.

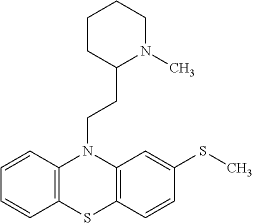 [SMILES: CSc1ccc2c(c1)N(CCC1CCCCN1C)c1ccccc1S2]

### Embodiment 6

Embodiment 1, 2 or 3, wherein the autophagy activator compound is at least one compound selected from the group consisting of:

(a) compounds of formula (I):

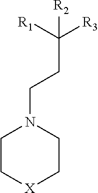

wherein X is selected from CR4R5 and NR6; R1 is selected from hydrogen, C1-6 alkyl and phenyl, wherein the alkyl and phenyl are substituted with 0 or 1 halogen; R2 is selected from hydrogen, C1-6 alkyl and phenyl, wherein the alkyl and phenyl are substituted with 0 or 1 halogen; R3 is selected from

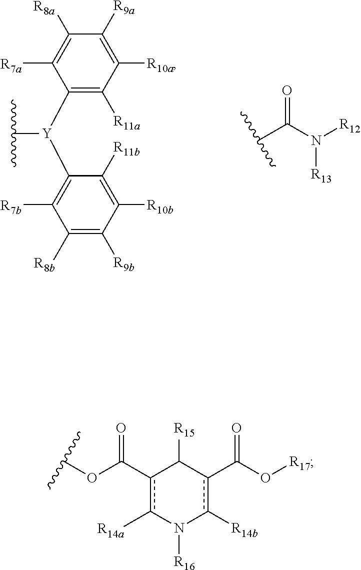

R4 is selected from hydrogen, hydroxyl, C1-6 alkyl and phenyl;
R5 is selected from C1-6 alkyl and phenyl, halophenyl, benzimidazole, dihydrobenzimidazole, benzimidazolone; optionally R4 and R5 are taken together to form a 5 or 6 membered heterocycloalkyl comprising two nitrogen atoms, wherein the heterocycloalkyl is substituted with 1, 2 or 3 substituents selected from the group consisting of C1-6 alkyl, phenyl, and ═O; R6 is selected from hydrogen and C1-6 alkyl; R7a, R8a, R9a, R10a, R11a, R7b, R8b, R9b, R10b, and R11b are each independently selected from hydrogen, hydroxyl, halogen and C1-6 haloalkyl; optionally R11a and R11b are taken together to form a heterocycle of the following structure:

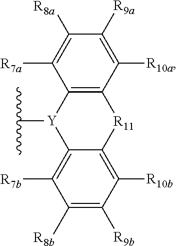

wherein R11 is selected from CH2, NH, O and S; R12 and R13 are each independently selected from hydrogen and C1-6 alkyl; R14a and R14b are each independently selected from hydrogen and C1-6 alkyl; R15 is selected from phenyl substituted with 0 or 1 halogen or nitro; R16 is selected from hydrogen and C1-6 alkyl; Y is N or CH; and pharmaceutically acceptable salts thereof; (b) compounds of formula (II):

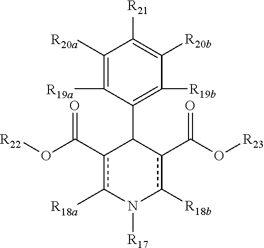

wherein R17 is selected from hydrogen and C1-6 alkyl; R18a and R18b, are each independently selected from hydrogen and C1-6 alkyl; R19a, R19b, R20a, R20b, and R21 are each independently selected from hydrogen, halogen and nitro; R22 is selected from hydrogen and C1-6 alkyl; R23 is selected from —(CH2)nNR24aR24b and —(CH2)nR24a; R24a and R24b are each independently selected from C1-6 alkyl and phenyl, wherein the alkyl is substituted with 0 or 1 phenyl substituents; optionally R24a and R24b are taken together with the nitrogen to which they are attached to form a piperidine, which is substituted with 0, 1 or 2 phenyl substituents; n is a positive integer from 2 to 4; and pharmaceutically acceptable salts thereof
(c) compounds of formula (III):

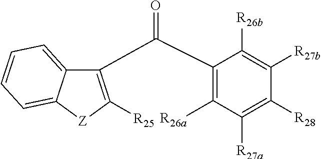

wherein R25 is selected from hydrogen and C1-6 alkyl; R26a, R26b, R27a, and R27b are each independently selected from hydrogen, halogen and C1-6 alkyl; R28 is selected from —O(CH2)mNR29aR29b and —NH(CH2)mNR29aR29b; R29a and R29b are each independently selected from hydrogen and C1-6 alkyl; Z is O, S or NH; m is a positive integer from 1 to 3; and pharmaceutically acceptable salts thereof
(d) compounds of formula (IV):

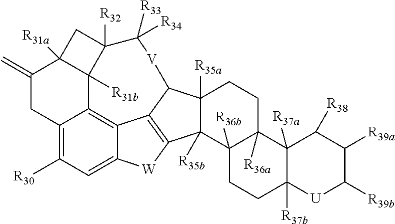

wherein R30 is selected from hydrogen, C1-6 alkyl and halogen; R31a and R31b are each independently selected from hydrogen, hydroxyl and C1-6 alkyl; R32 is selected from hydrogen, hydroxyl and C1-6 alkyl; R33 and R34 are each independently selected from hydrogen and C1-6 alkyl; R35a and R35b are each independently selected from hydrogen, hydroxyl and C1-6 alkyl; R36a and R36b are each independently selected from hydrogen, hydroxyl and C1-6 alkyl; R37a and R37b are each independently selected from hydrogen, hydroxyl and C1-6 alkyl; R38 is selected from hydrogen, hydroxyl and C1-6 alkyl; optionally R37a and R38 are taken together to form a three membered heterocycle of the formula:

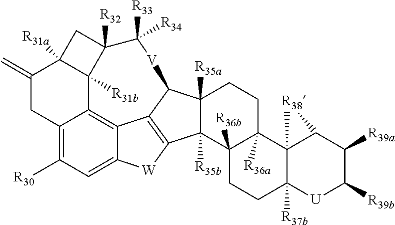

wherein R38′ is O, S or NH; R39a is selected from hydrogen, hydroxyl and C1-6 alkyl; R39b is selected from hydrogen, hydroxyl, C1-6 alkyl and C2-6 alkenyl; U, V and W are each independently selected from O, S, and NH; and pharmaceutically acceptable salts thereof.

### Embodiment 7

Any one of embodiments 1 to 3, wherein the autophagy activator compound is Azithromycin macrolide (an azalide, a subclass of macrolide antibiotics, IUPAC name: 2R,3S,4R,5R,8R,10R,11R,12S,13S,14R)-2-ethyl-3,4,10-trihydroxy-3,5,6,8,10,12,14-heptamethyl-15-oxo-11-{[3,4,6-trideoxy-3-(dimethylamino)-β-D-xylo-]oxy}-1-oxa-6-azacyclopentadec-13-yl 2,6-dideoxy-3-C-methyl-3-O-methyl-α-L-ribo-hexopyranoside).

### Embodiment 8

Any one of embodiments 1 to 3, wherein the autophagy activator compound is Loperamide.

### Embodiment 9

Any one of embodiments 1 to 3, wherein the autophagy activator compound is Silibinin.

### Embodiment 10

Any one of embodiments 1 to 3, wherein the autophagy activator compound is Niguldipine.

### Embodiment 11

Any one of embodiments 1 to 3, wherein the autophagy activator compound is Pimozide.

### Embodiment 12

Any one of embodiments 1 to 3, wherein the autophagy activator compound is Nicardipine.

### Embodiment 13

Any one of embodiments 1 to 3, wherein the autophagy activator compound is Penitrem A.

### Embodiment 14

Any one of embodiments 1 to 3, wherein the autophagy activator compound is Fluspirilene.

### Embodiment 15

Any one of embodiments 1 to 3, wherein the autophagy activator compound is Trifluoperazine.

### Embodiment 16

Any one of embodiments, wherein the autophagy activator compound is 10-NCP.

### Embodiment 17

Any one of embodiments 1 to 3, wherein the autophagy activator compound is Promazine.

### Embodiment 18

Any one of embodiments 1 to 3, wherein the autophagy activator compound is Chloropromazine.

### Embodiment 19

Any one of embodiments 1 to 3, wherein the autophagy activator compound is Triflupromazine.

### Embodiment 20

Any one of embodiments 1 to 3, wherein the autophagy activator compound is Prometazine.

### Embodiment 21

Any one of embodiments 1 to 3, wherein the autophagy activator compound is Mesoridazine.

### Embodiment 22

Any one of embodiments 1 to 3, wherein the autophagy activator compound is Thioridazine.

### Embodiment 23

Any one of embodiments 1 to 22, wherein the treatment is to inhibit increased bone resorption disorder.

### Embodiment 24

Any one of embodiments 1 to 22, wherein the treatment is to inhibit osteoclast activity.

### Embodiment 25

Any one of embodiments 1 to 22, wherein the treatment is to inhibit formation of osteoclasts (TRAP-positive multinuclear cells) from blood progenitor cells.

### Embodiment 26

Any one of embodiments 1 to 22, wherein the treatment is to activate bone formation.

### Embodiment 27

Any one of embodiments 1 to 22, wherein the bone degenerative disorders are bone metabolism disorders.

### Embodiment 28

Any one of embodiments 1 to 22, wherein the bone degenerative disorder is caused by acute critical illness or prolonged critical illness.

### Embodiment 29

Any one of embodiments 1 to 22, wherein the bone degenerative disorder is caused by (parenteral) nutrition-induced suppression of autophagy.

### Embodiment 30

Any one of embodiments 1 to 22, wherein the bone degenerative disorder is caused by mTOR activation.

### Embodiment 31

Any one of embodiments 1 to 22, wherein the bone degenerative disorders are hyperresorption of bone.

### Embodiment 32

Any one of embodiments 1 to 22, wherein the bone degenerative disorders are an imbalance in the regulation of bone resorption and bone formation results in metabolic bone diseases, such as, osteoporosis.

### Embodiment 33

Any one of embodiments 1 to 22, wherein the bone degenerative disorders are elderly osteoporosis.

### Embodiment 34

Any one of embodiments 1 to 22, wherein the bone degenerative disorders are osteoporosis.

### Embodiment 35

Any one of embodiments 1 to 22, wherein the bone degenerative disorders are pathological increased bone resorption and pathological decreased bone formation.

### Embodiment 36

Any one of embodiments, wherein the bone degenerative disorder is disrupted calcium and bone metabolism by acute critical illness and/or prolonged critical illness.

### Embodiment 37

Any one of embodiments 1 to 22, wherein the bone degenerative disorders are caused by enhanced osteoclast formation from circulating precursors in the blood during critical illness.

### Embodiment 38

Any one of embodiments 1 to 22, wherein the bone degenerative disorders are increased osteoclast formation from circulating precursors leading to osteoporosis.

### Embodiment 39

Any one of embodiments 1 to 22, wherein the bone degenerative disorders are increased osteoclast formation due to a increased cytokine production disorder.

### Embodiment 40

Any one of embodiments 1 to 22, wherein the bone degenerative disorders are increased osteoclast formation due to hormonal disorder.

### Embodiment 41

Any one of embodiments 1 to 22, wherein the bone degenerative disorders are increased osteoclast formation due to Vitamin D deficiency.

### Embodiment 42

Any one of embodiments 1 to 22, wherein the bone degenerative disorders are increased osteoclast formation due to glucocortoids (pre)treatment.

### Embodiment 43

Any one of embodiments 1 to 22, wherein the bone degenerative disorders are increased osteoclast formation due to heparinoids (pre)treatment.

### Embodiment 44

Any one of embodiments 1 to 22, wherein the bone degenerative disorders are increased osteoclast formation due to autophagy deficiency disorder.

### Embodiment 45

Any one of embodiments 1 to 22, wherein the bone degenerative disorders are osteoclast activity increase due to acute critical illness and/or prolonged critical illness.

### Embodiment 46

Any one of embodiments 1 to 22, wherein the bone degenerative disorders are osteoclast activity increase due to increased cytokine production disorder.

### Embodiment 47

Any one of embodiments 1 to 22, wherein the bone degenerative disorders are osteoclast activity increase due to hormonal disorder.

### Embodiment 48

A therapeutic agent or a pharmaceutical composition comprising an autophagy activator compound according to any one of the previous embodiments or an analogue, thereof, for use in treating or preventing bone degenerative disorder in a mammalian subject.

### Embodiment 49

Embodiment 48, wherein the bone degenerative disorder is increased bone resorption disorder or bone hyperresorption.

### Embodiment 50

Embodiment 48, wherein the treatment is to prevent, inhibit or reduce increased bone resorption or bone hyperresorption.

Particular and preferred aspects hereof are set out in the accompanying independent and dependent claims. Features from the dependent claims may be combined with features of the independent claims and with features of other dependent claims as appropriate and not merely as explicitly set out in the claims.

### BRIEF DESCRIPTION OF THE DRAWINGS

The invention will become more fully understood from the detailed description given herein below and the accompanying drawings, which are given by way of illustration only, and thus are not limitative of the disclosure, and wherein:

FIG. 1 concerns graphics (B) showing spermidine dose response—osteoclast activity (Resorption) in stimulated healthy Peripheral Blood Mononuclear Cells (PBMCs) and shows the photos (A) on the osteoclast PBMCs with spermidine. Healthy PBMCs were differentiated for 14 days in the presence of RANKL and M-CSF, with or without spermidine (2×10⁻⁶ M-2×10⁻⁹ M), on a hydroxyapatite film layer. At 14 days, cells were removed with sodium hypochlorite, and the hydroxyapatite layer was stained with Von Kossa. Osteoclast resorption was analyzed using Image J software. (A)Von Kossa staining of the hydroxyapatite film layer. (B) Percentage of total area resorbed. (**p≦0.01 vs. Control; *p≦0.05 vs. Control). As the lowest concentration still affecting osteoclast resorption, 2×10-8 M spermidine was selected for use in future experiments.

FIG. 2 demonstrates the effect of spermidine osteoclast formation in stimulated (+RANKL/MCSF) PBMCs. FIG. 2A is a photodisplay and FIG. 2B a graphic on mature osteoclast formation in healthy and sick stimulated PBMCs with spermidine. Healthy and sick PBMCs were differentiated for 14 days in the presence of RANKL and M-CSF, with or without spermidine (2×10-8 M). At 14 days, cells were stained for tartrate-resistant acid phosphatase (TRAP), and nuclei visualized with DAPI staining. Mature osteoclasts were identified as cells staining positively for TRAP, with 3 or more nuclei. (A) Micrograph images of PBMCs in culture at day 7, and at day 14 following TRAP/DAPI staining (B) Number of TRAP positive multinuclear cells per 5×10⁵ cells plated (***p≦0.001 vs. Healthy cells; αp≦0.05 vs. sick cells; n=4)

FIG. 3 concerns the effects of spermidine on spontaneous osteoclast formation in unstimulated PBMCs. FIG. 3 demonstrates spontaneous mature osteoclast formation in healthy and sick unstimulated PBMCs with spermidine. Healthy and sick PBMCs were cultured for 14 days with or without spermidine (2×10-8 M). At 14 days, cells were stained for tartrate-resistant acid phosphatase (TRAP), and nuclei visualized with DAPI staining. Mature osteoclasts were identified as cells staining positively for TRAP, with 3 or more nuclei. (A) Micrograph images of PBMCs at day 14 following TRAP/DAPI staining (B) Number of TRAP positive multinuclear cells per 5×10⁵ cells plated (***p≦0.001 vs. Healthy cells; αp≦0.05 vs. sick cells; n=4)

FIG. 4 concerns the effect of spermidine on osteoclast activity in stimulated (+RANKL/MCSF) PBMCs. FIG. 4, Osteoclast activity in stimulated healthy and sick PBMCs with spermidine. Healthy and sick PBMCs were cultured for 14 days in the presence of RANKL and M-CSF with or without spermidine (2×10⁻⁸M) on a hydroxyl apatite film layer. At 14 days, cells were removed with sodium hypochlorite, and the hydroxyapatite layer was stained with VonKossa. Osteoclast resorption was analyzed using Image J software. (A) Von Kossastaining of the hydroxyapatite film layer. (B) Percentage of total area resorbed. (***p≦0.01 vs. Healthy; αp≦0.05 vs. sick; n=4)

FIG. 5 concerns the effect of spermidine on spontaneous osteoclast activity in unstimulated PBMCs. Healthy and sick PBMCs were cultured for 14 days with or without permidine (2×10⁻⁸ M) on a hydroxyl apatite film layer. At 14 days, cells were removed with sodium hypochlorite, and the hydroxyapatite layer was stained with VonKossa. Osteoclast resorption was analyzed using Image J software. (A) Von Kossa staining of the hydroxyapatite film layer. (B) Percentage of total area resorbed.

FIG. 6 concerns autophagy marker p62 protein expression in healthy and sick PBMCs/osteoclasts (RANKL/MCSF stimulated) and the effect of spermidine on healthy PBMCs/osteoclasts. Healthy and sick PBMCs were cultured for 14 days with or without spermidine (2×10⁻⁸ M) in 6 well plates (5×10⁶ cells/well). After 14 days, protein was extracted using the Ambion PARIS extraction kit (Applied Biosystems), and proteins quantified using the BC Protein Assay (Thermo Fischer Scientific). (A) 5 ug of protein was subjected to 15% Bis-tris gelelectrophoresis, transferred to nitrocellulose membrane and incubated at 4° C. overnight in Anti-p62 (Novus Biologicals; 1:1000). p62 protein was detected by ECL following incubation with AntiMouseIgG (DAKO; 1:1000). GAPDH was used as a loading control. (B) p62 relative expression was quantified with Image QuantTL (Amersham Biosciences) and normalized to GAPDH (n=3; ***p≦0.05 vs. Healthy).

FIG. 7 Demonstrates that Osteoblast markers, such as, bone specific alkaline phosphatase also decrease over time in ICU. bsALP=Serum Bone Specific Alkaline phosphatase (bsALP).

FIG. 8 demonstrates effects on Osteoclast markers, such as, Crosslaps increase over time in ICU. Serum Crosslaps (collagen cross links).

FIG. 9 providers a graphic overview of provides a review on osteoclast formation from circulating precursors in the blood, osteoclast maturation, osteoclast bone resorption activity and the current marks with are indicative for such.

FIG. 10a demonstrates osteoclast formation in PBMCs from Critically Ill Patients. Osteoclast formation is observed in Patient PBMCs after only 7 days in culture with RANKL and MCSF.

FIG. 10b demonstrates the osteoclast formation in PBMCs from Critically Ill Patients. By Day 21 osteoclast formation is observed in Patient PBMCs with and without RANKL/M-CSF.

FIG. 10c shows the Day 21 Patient PBMCs with and without RANKL/MCSF are multinuclear.

FIG. 11 shows mTOR-dependent autophagy inducers.

FIG. 12 shows mTOR-independent autophagy inducers.

FIG. 13 shows additional mTOR-independent autophagy inducers.

FIG. 14 displays a dose response of spermidine on osteoclast formation.

FIG. 15 displays a dose response of rapamycin on osteoclast formation.

FIG. 16 displays a dose response of Everolimus on osteoclast formation.

FIG. 17 displays a dose response of prometazine on osteoclast formation.

FIG. 18 displays a dose response of 5-Azacytidine on osteoclast formation.

FIG. 19. Rabbit in vivo study of prolonged critical illness (A) Serum ionized calcium levels were significantly lower in critically ill rabbits compared to healthy controls over the 7-day period (n=30 critically ill (grey boxes) and 15 healthy (white boxes); **p<0.01; multiple testing ANOVA). (B) Serum osteocalcin levels were significantly reduced (62.3%) in critically ill rabbits at day 4 of illness (n=30 critically ill and 15 healthy; **p<0.01). (C) Trabecular BMC and (D) BMD were significantly reduced (30.6% and 28.8%, respectively) in the proximal tibiae of critically ill rabbits vs. healthy controls. (E) No significant difference in cortical BMC or (F) thickness was observed (n=30 and 15, respectively; *p<0.05, **p<0.01).

FIG. 20. Circulating osteoclast precursors in healthy and critically ill patient peripheral blood (A) Representative dot plots of CD14+/CD11b+ early osteoclast precursors and CD14+/CD11b+/VNR+ osteoclast precursors in healthy and patient peripheral blood. (B) FACS analysis revealed a significant increase in early osteoclast precursors (CD14/CD11b positive) from the peripheral blood of critically ill patients (99.8±0.38% and 83.9±7.09%, respectively). A trend towards an increase in mature circulating osteoclasts (CD14/CD11b/VNR positive) was also observed (n=5; **p<0.01).

FIG. 21. Human in vitro study of osteoclast differentiation (A) Formation of mature, multi-nuclear osteoclasts was visualized by TRAP positivity (scale bar=100 μm) (I) and resorption on hydroxyapatite (scale bar=500 μm) (II) in healthy or patient cultures, in the presence and absence of RANKL and M-CSF. (B) The number of TRAP positive multi-nuclear cells was significantly increased in critically ill patient PBMC cultures with and without RANKL and M-CSF. (C) Resorption of hydroxyapatite was increased in patient cultures in the presence and absence of RANKL and M-CSF. (n=8; ***p<0.05 versus healthy cells).

FIG. 22. Effect of critically ill patient serum on osteoclast differentiation and activity (A) Formation of mature, multi-nuclear osteoclasts was visualized by TRAP positivity (top row), F-actin ring formation (second row), formation of multi-nuclear cells (third row, merged image of TRAP, DAPI and F-actin ring staining) and resorption on hydroxyapatite (bottom row; analyzed with Von Kossa staining) in pooled PBMC cultures from healthy controls and critically ill patients. (B) The number of TRAP positive multi-nuclear cells was significantly increased in patient PBMC cultures with (white bars) and without (grey bars) osteoclastogenic factors, in the presence of PS. (C) F-actin ring formation was also significantly increased in the presence of patient serum in patient PBMC cultures in the presence (white bars) and absence (grey bars) of RANKL and MCSF. (D) Resorption of hydroxyapatite was increased in patient cultures cultured with 10% HS or 10% PS in the presence (white bars) and absence (grey bars) of RANKL and MCSF (n=8; ***p<0.05 versus patient cells plus HS and healthy cells plus PS; αp<0.05 versus healthy cells plus HS).

FIG. 23. Inhibition of inflammatory cytokines in patient serum increases in vitro osteoclast formation. (A) Formation of mature, multinuclear osteoclasts was visualized by TRAP in pooled PBMC cultures from critically ill patients with 10% PS and varying concentrations of Anti-IL-6 and/or Anti-TNF-α neutralizing antibodies. (B) The number of TRAP positive multi-nuclear cells was significantly increased in patient PBMC cultures with 1 μg/ml Anti-IL-6 or 4 μg/ml and 8 μg/ml of Anti-TNF-α, or a combination of Anti-IL-6 (1 μg/ml) and Anti-TNF-α (8 m/ml) (n=4; *p<0.05, ***p<0.001 vs. control cultures).

FIG. 24. Effect of critically ill patient serum on osteogenic differentiation and angiogenesis in vitro (A) Alizarin Red staining of hPDC monolayers cultured for 21 days in 10% FBS, 10% HS or 10% PS. (B) Alizarin Red staining quantified by assessment of optical density at 540 nm revealed no significant difference in mineralization. (C) qRT-PCR analysis of RUNX2; (D) COL1A1; (E) ALP; and (F) VEGFA normalized to β-actin revealed no differences in gene expression between HS and PS conditions, although a reduction in VEGF-R1 expression was observed in patient serum conditions (G), which was also observed at the protein level (H) (n=8; ***p<0.001 versus FBS; **p<0.01 versus FBS; αp<0.01 versus healthy serum).

FIG. 25. Effect of critically ill patient serum on bone formation in vivo. (A) Bone quantification of hPDC NUOSS™ implants was carried out using μCT analysis 8 weeks after implantation, and revealed a significant reduction in bone formation in patient-serum conditions compared to HS (mature bone=yellow; remaining CaP grains=blue). No significant differences in CaP grains or fibrous tissue were detected in the scaffold. (B) A trend towards an increase in the number of TRAP positive osteoclasts per scaffold in patient serum-treated hPDCs was observed, although this did not reach significance (p=0.07). (C) Upon explant, scaffolds coated with PS treated hPDCs had visibly less vasculature than scaffolds coated with HS hPDCs (insets). This was confirmed by quantification of CD31 immunohistochemistry, with a significant reduction in the number of CD31 positive blood vessels in scaffolds with PS treated hPDCs (n=4; ***p<0.001 versus healthy serum; αp<0.01 versus FBS).

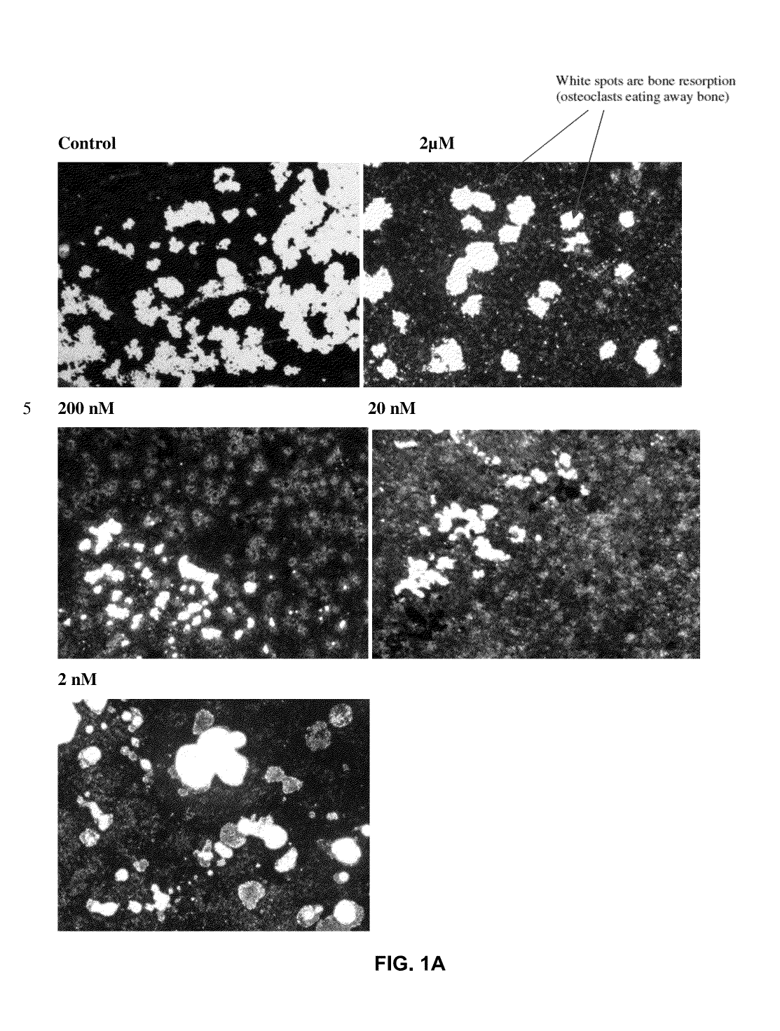

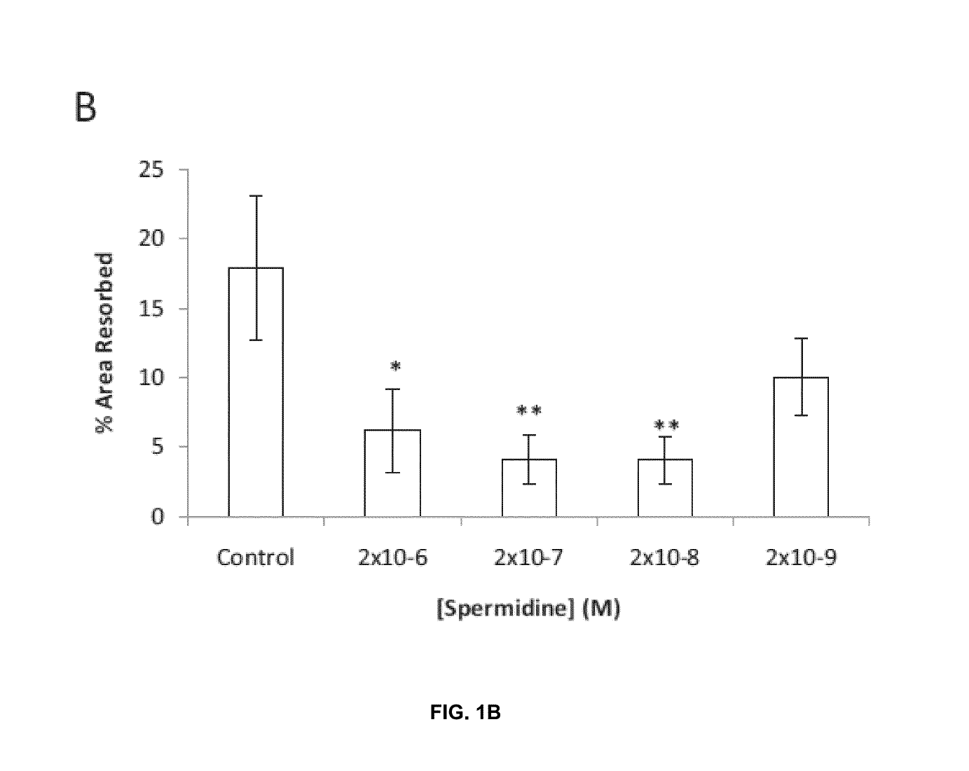

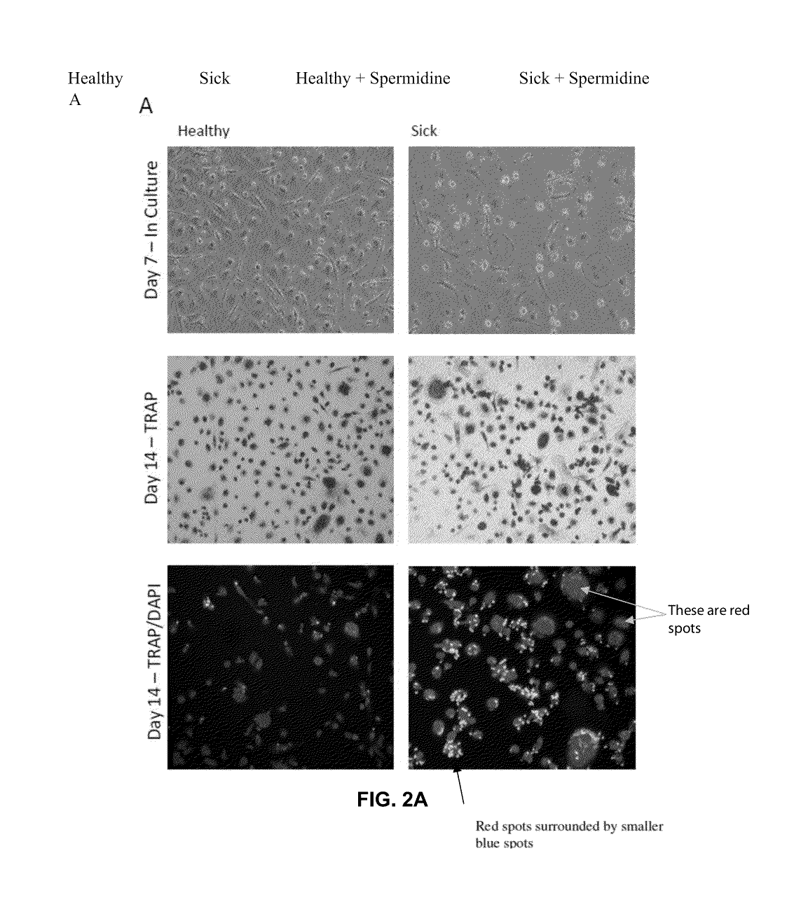

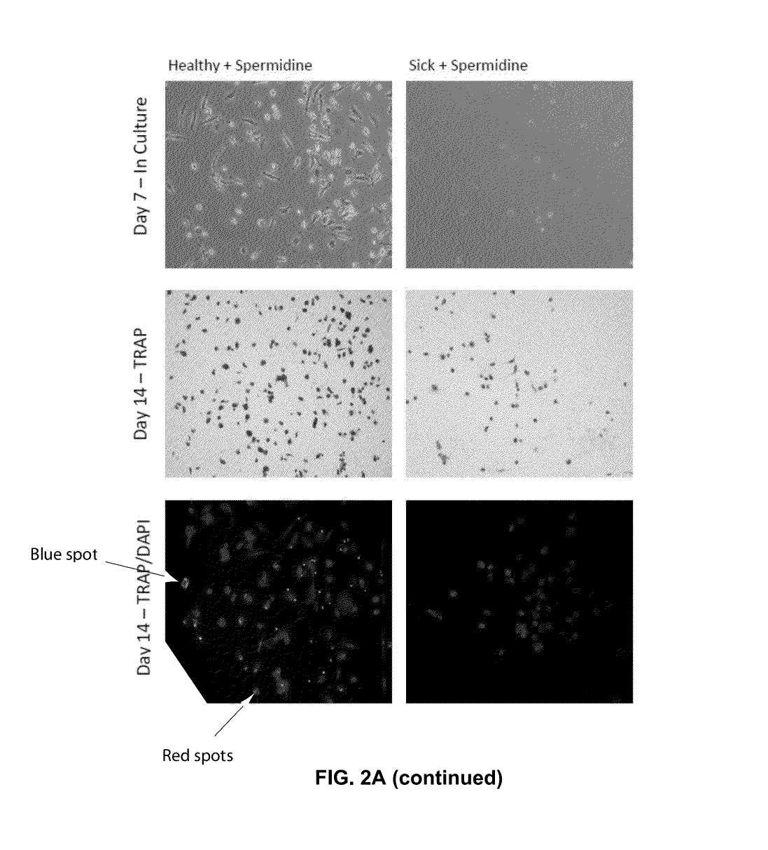

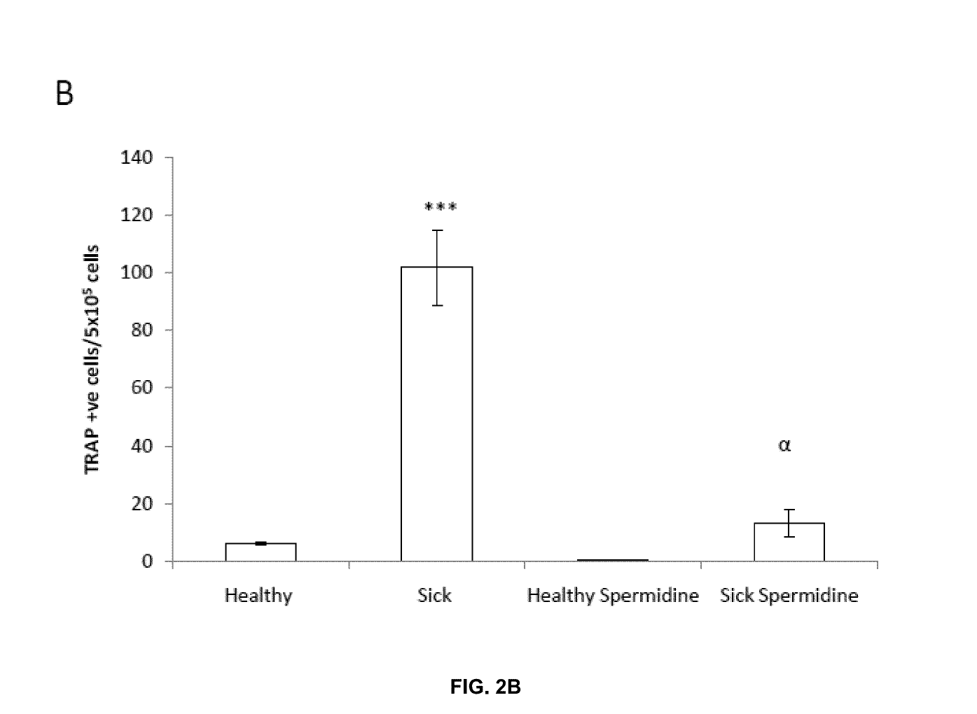

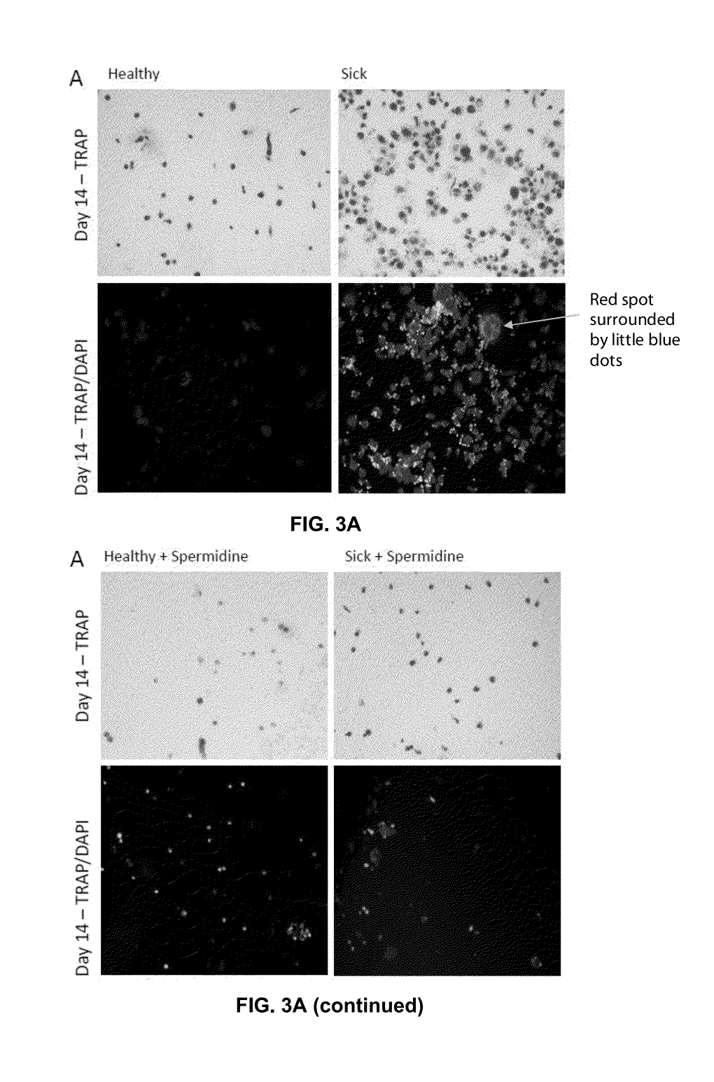

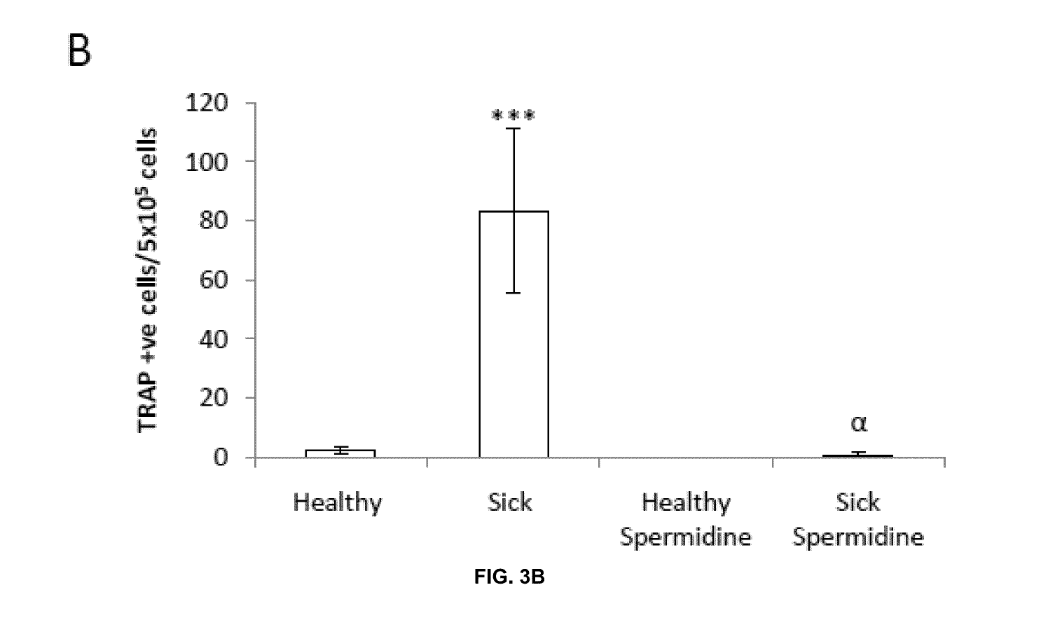

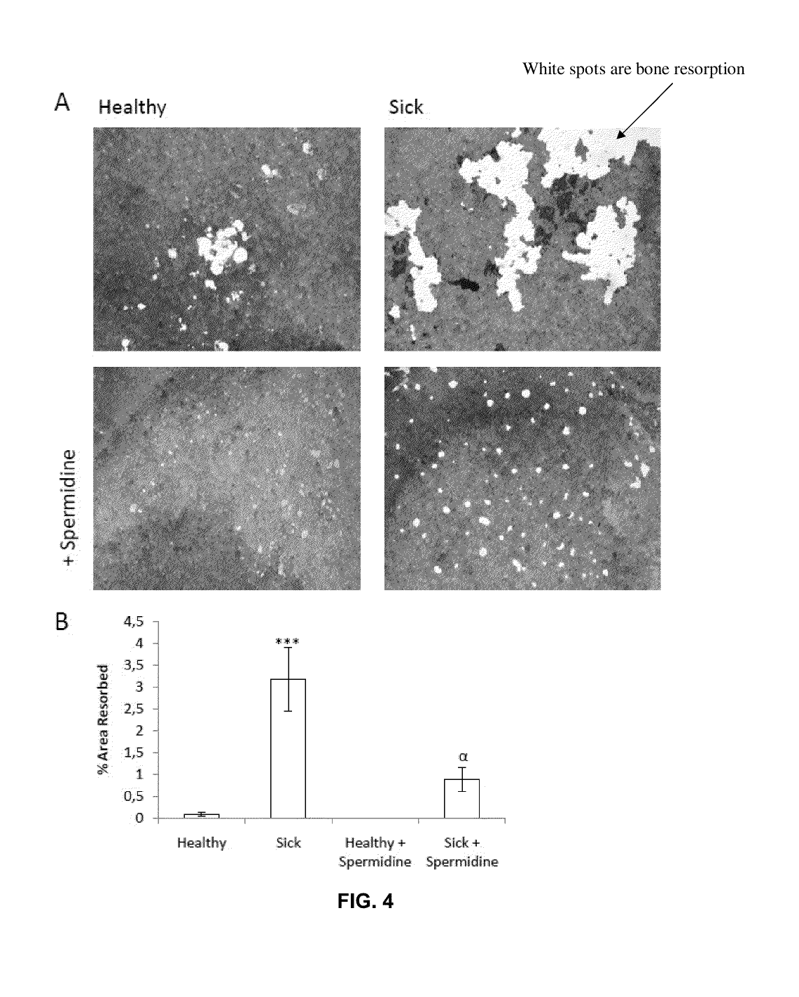

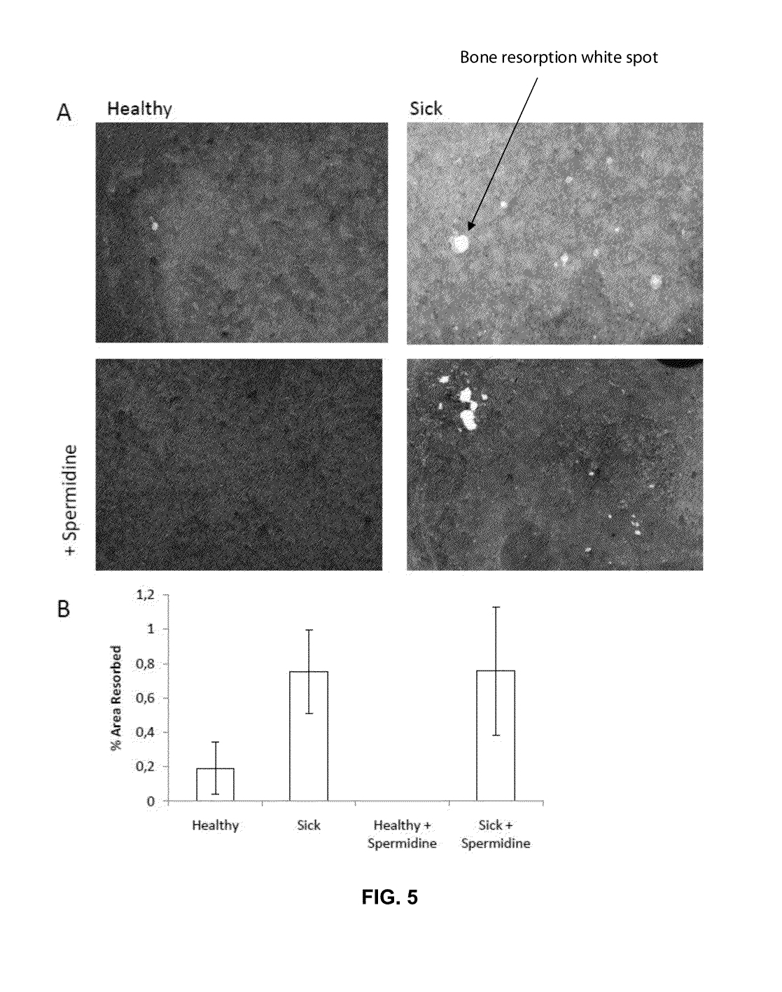

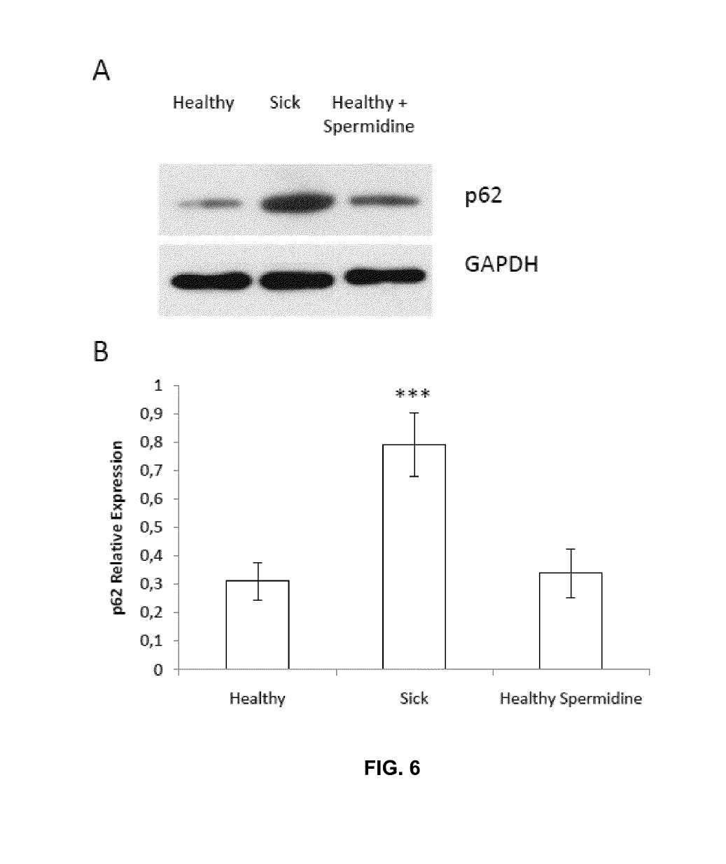

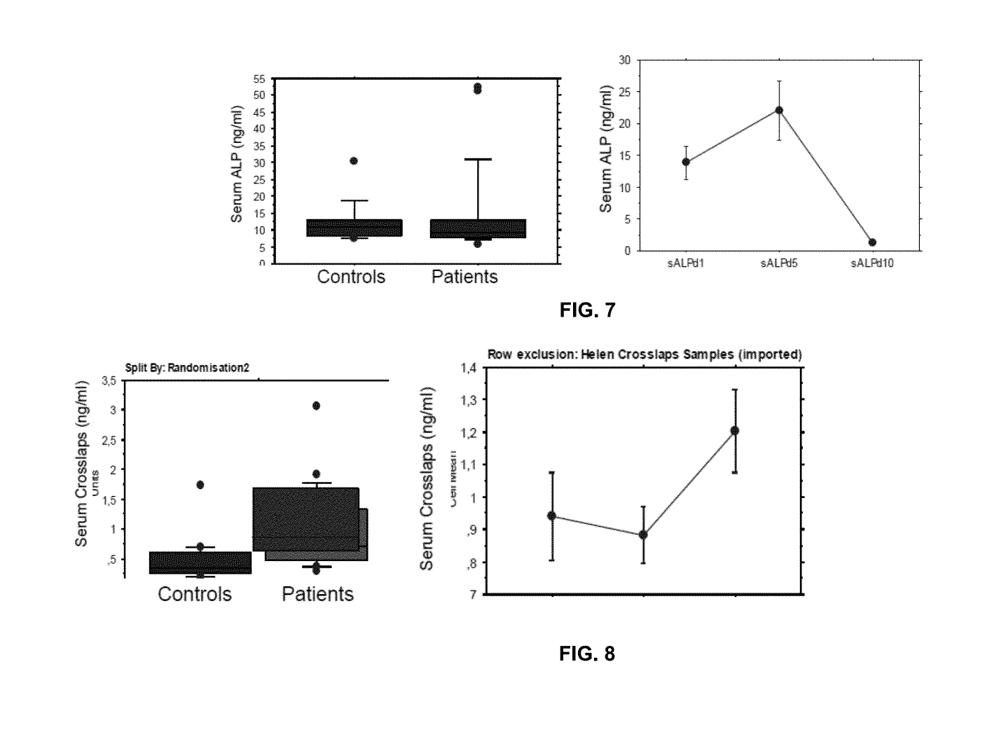

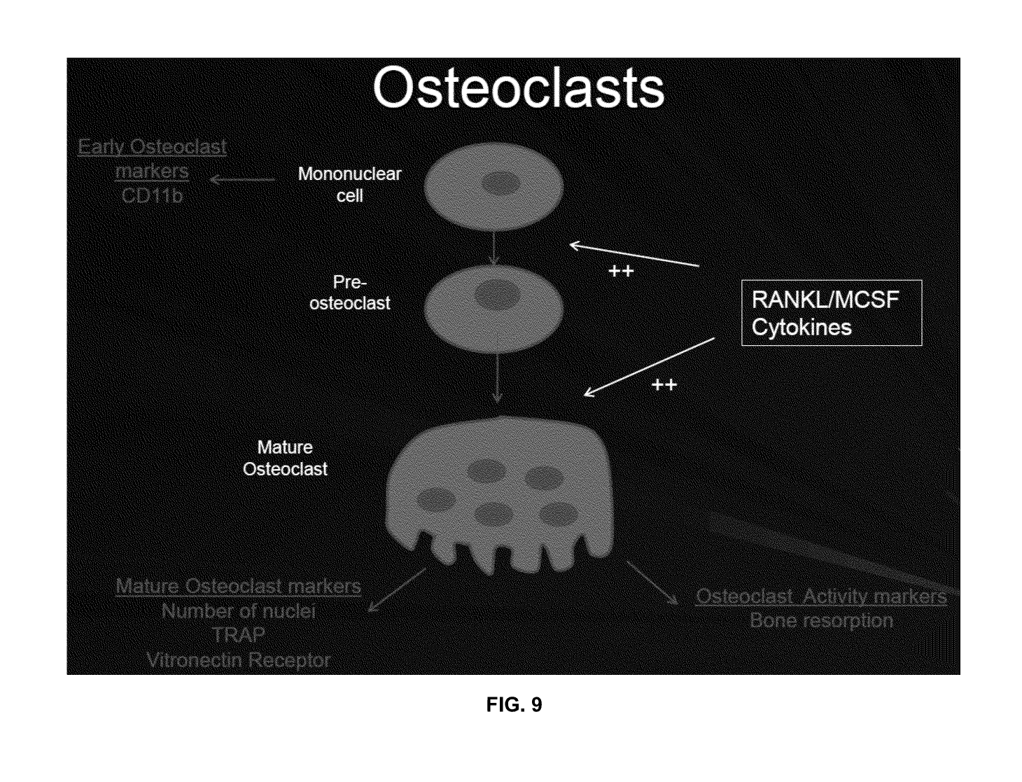

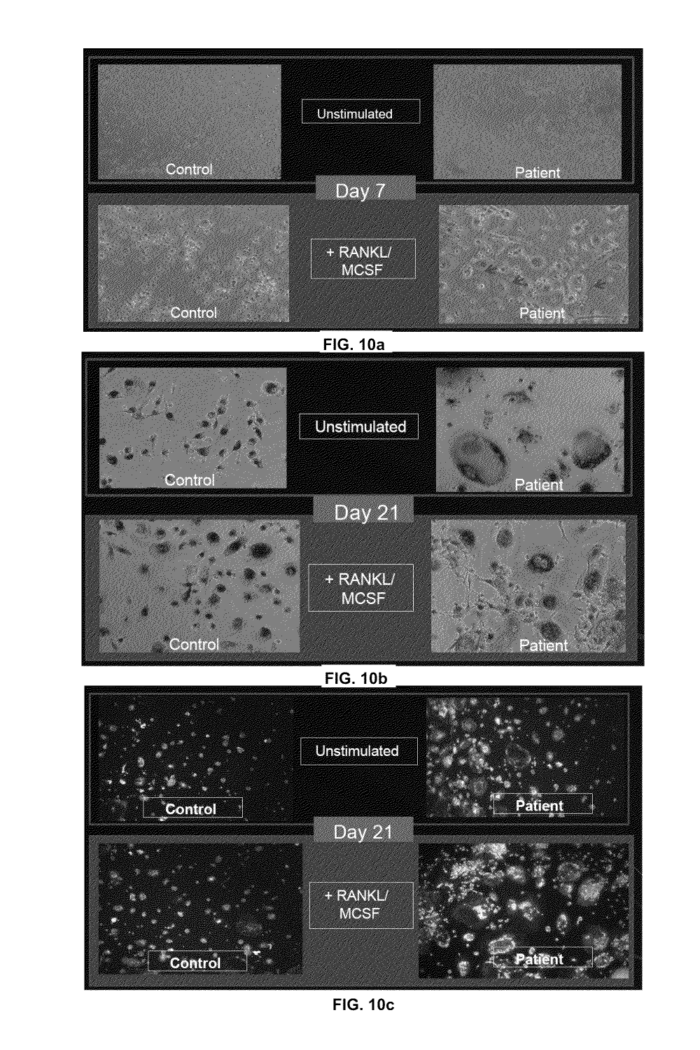

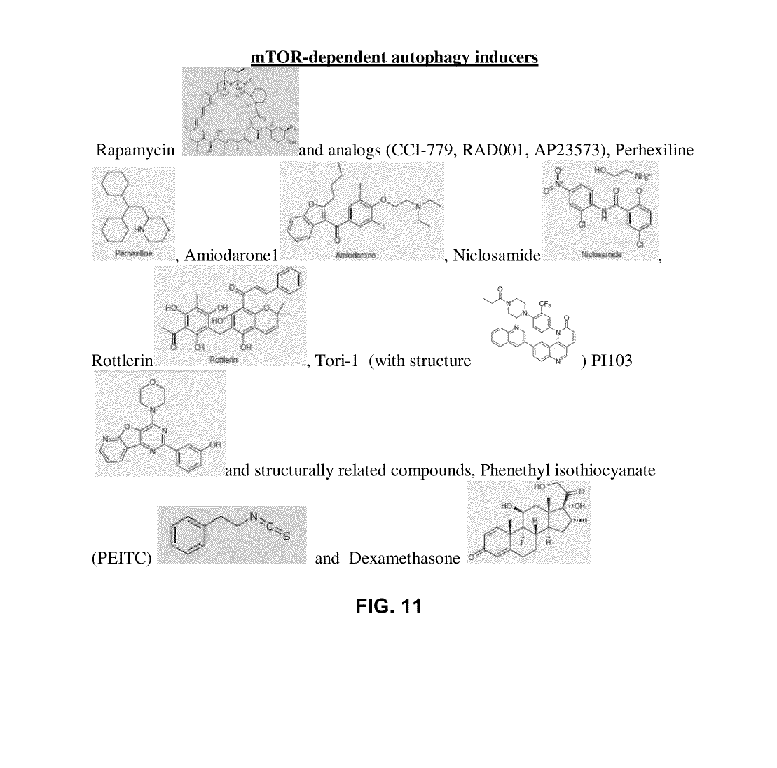

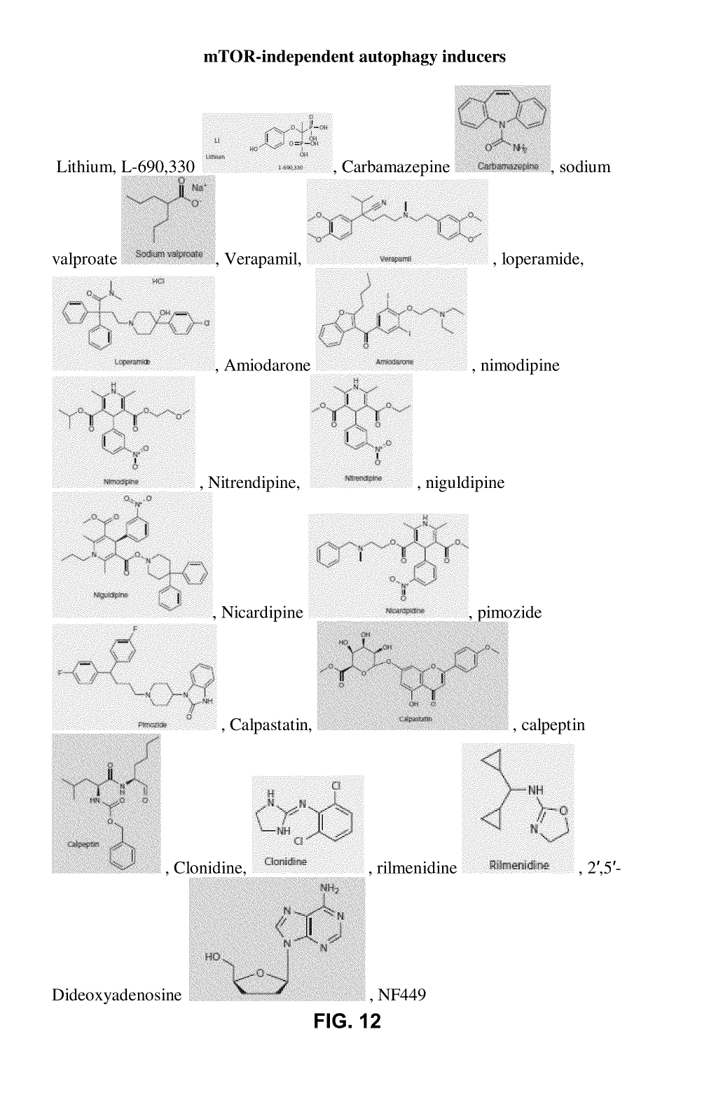

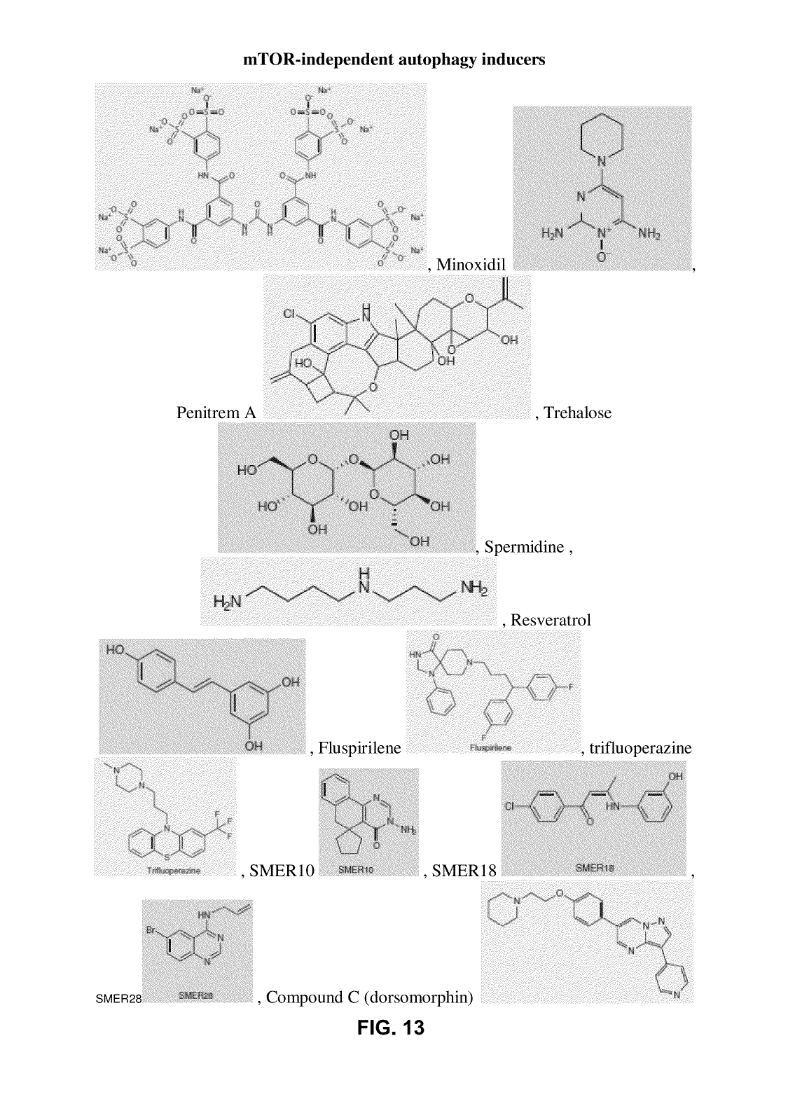

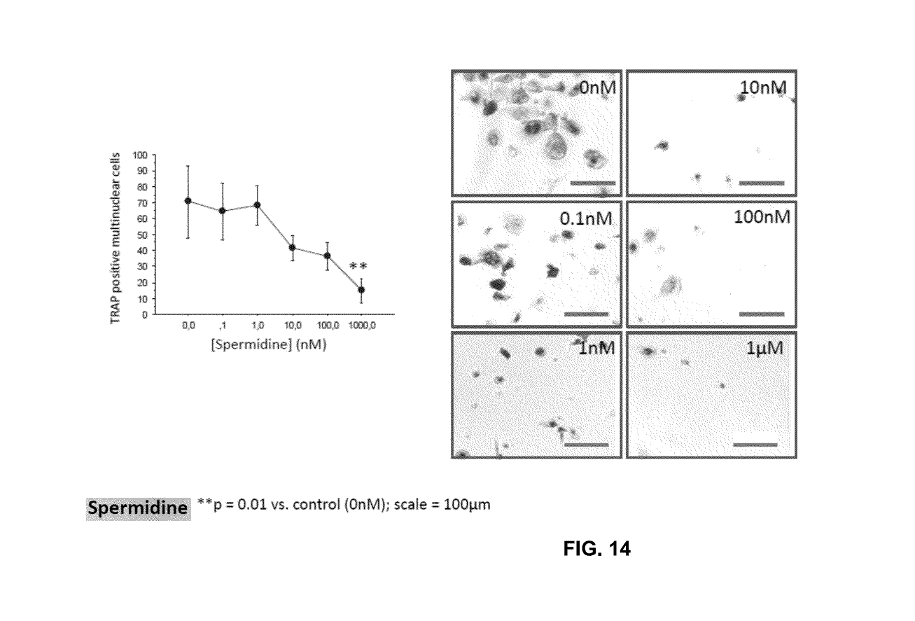

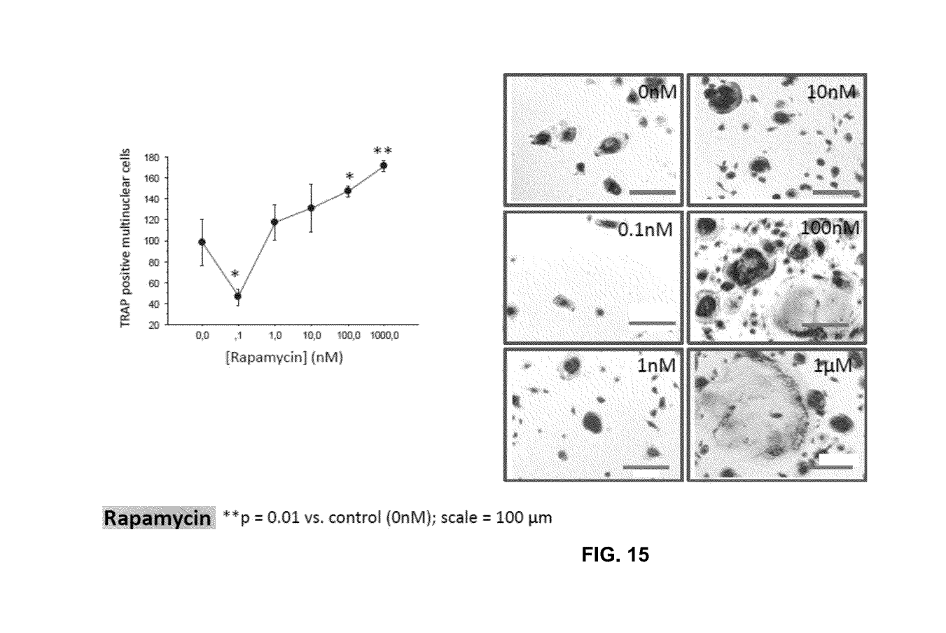

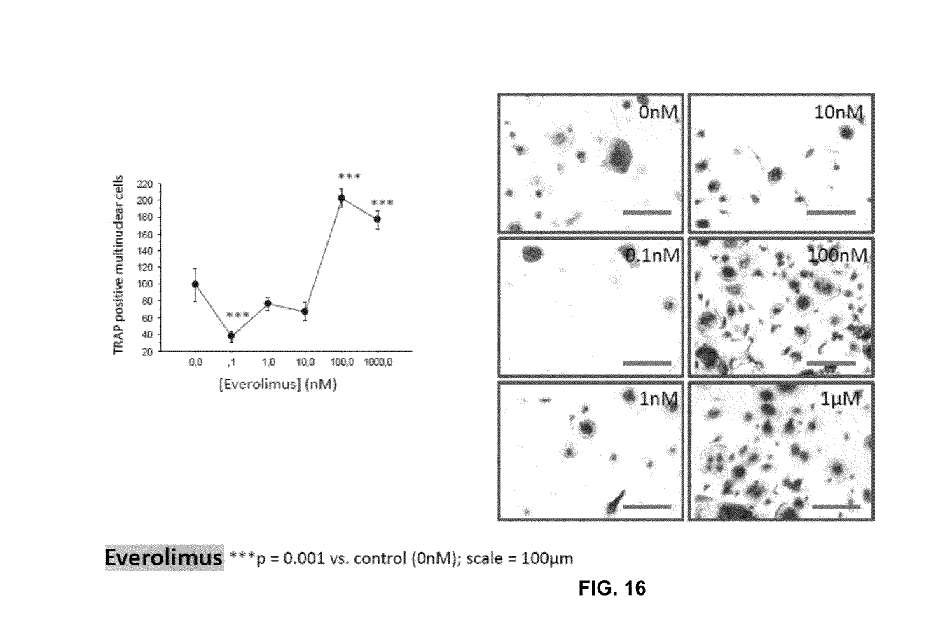

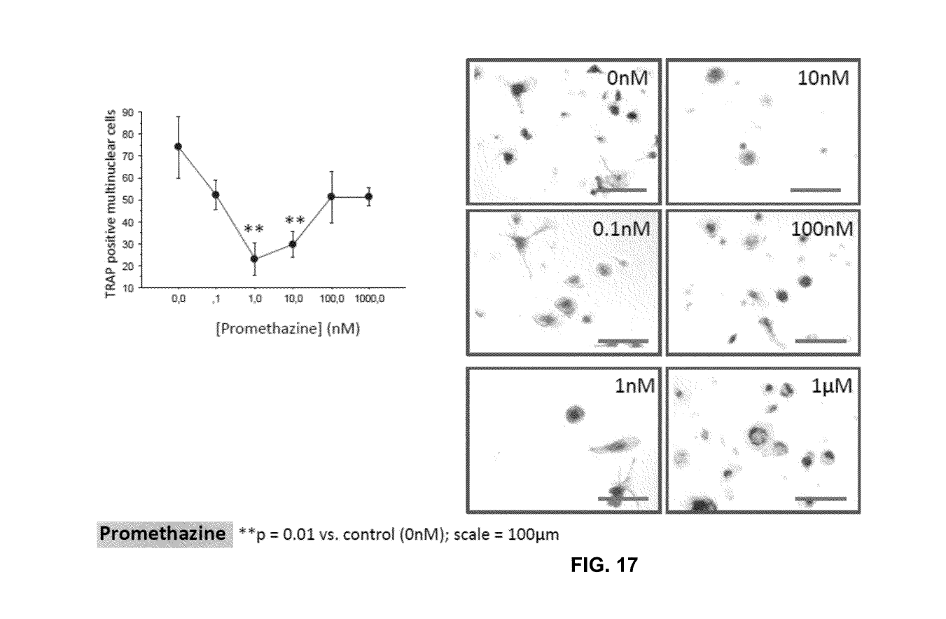

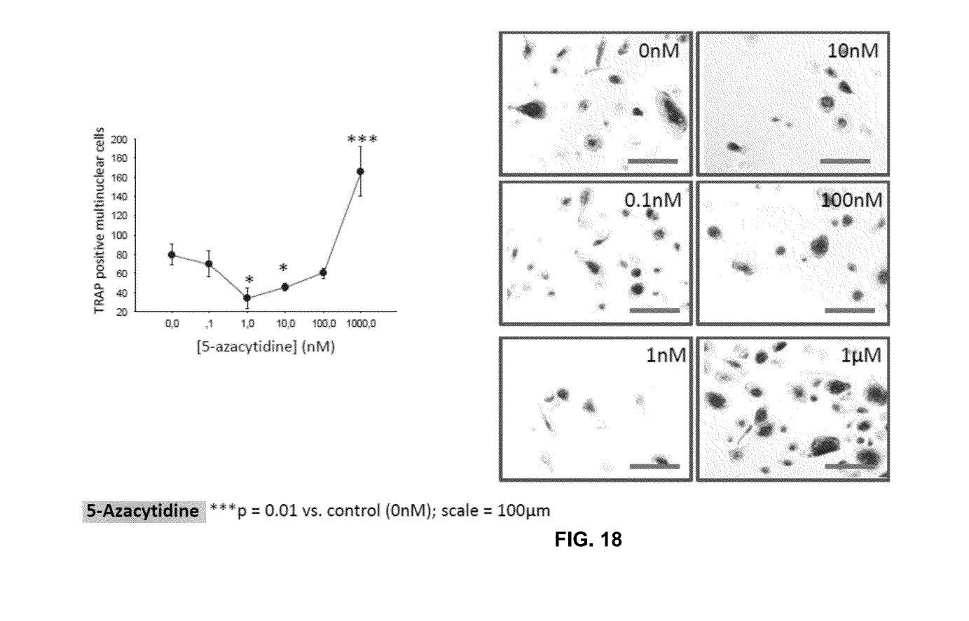

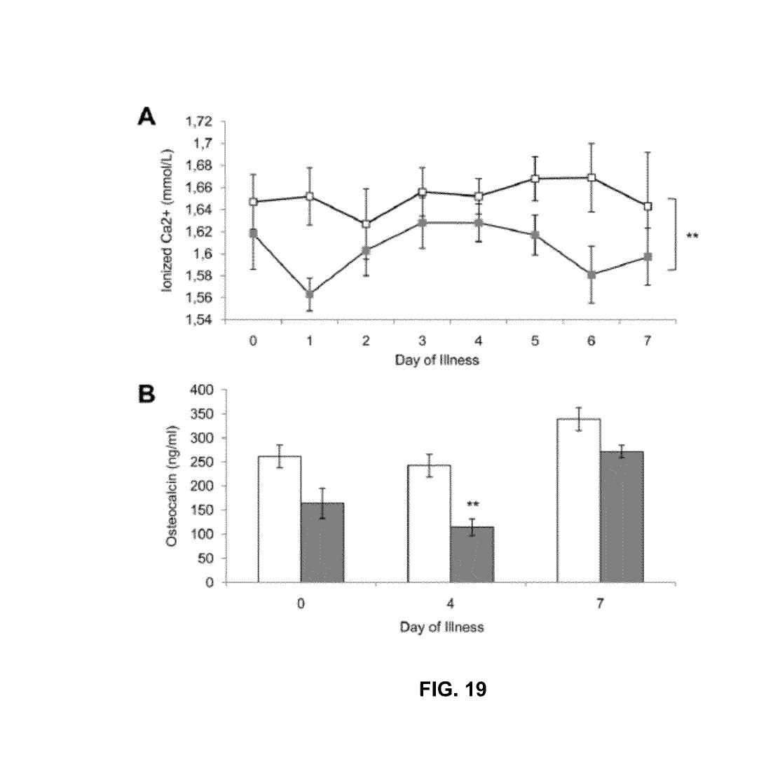

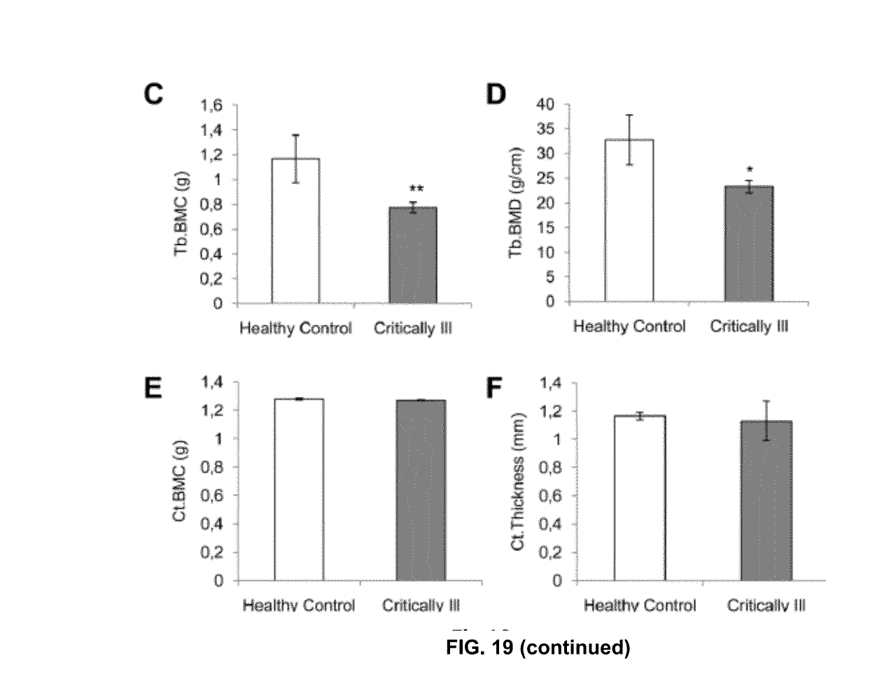

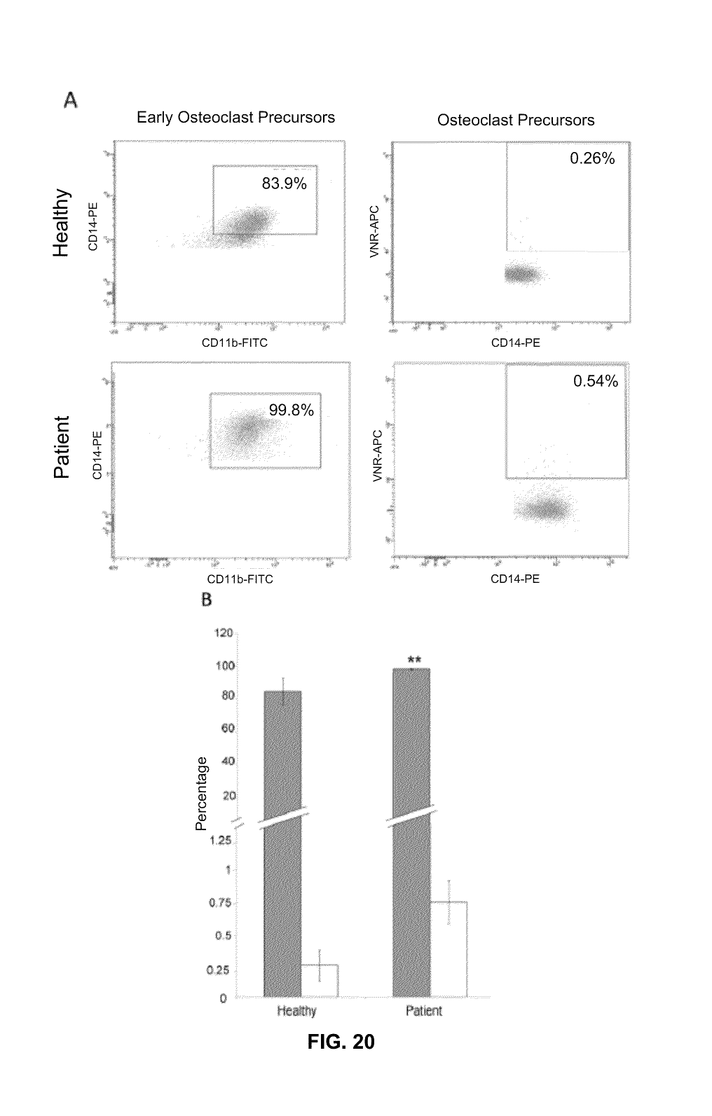

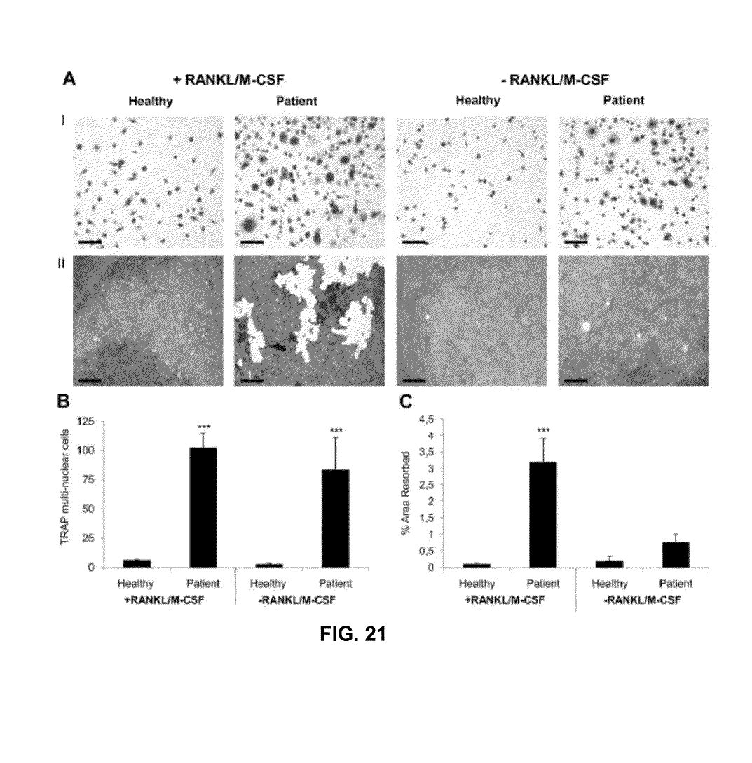

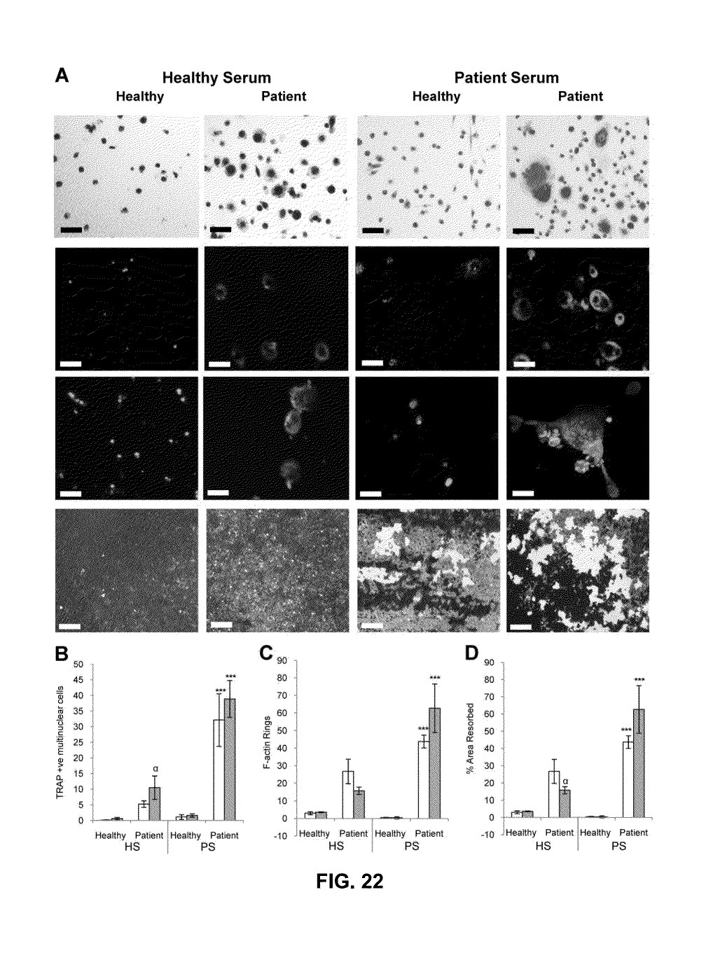

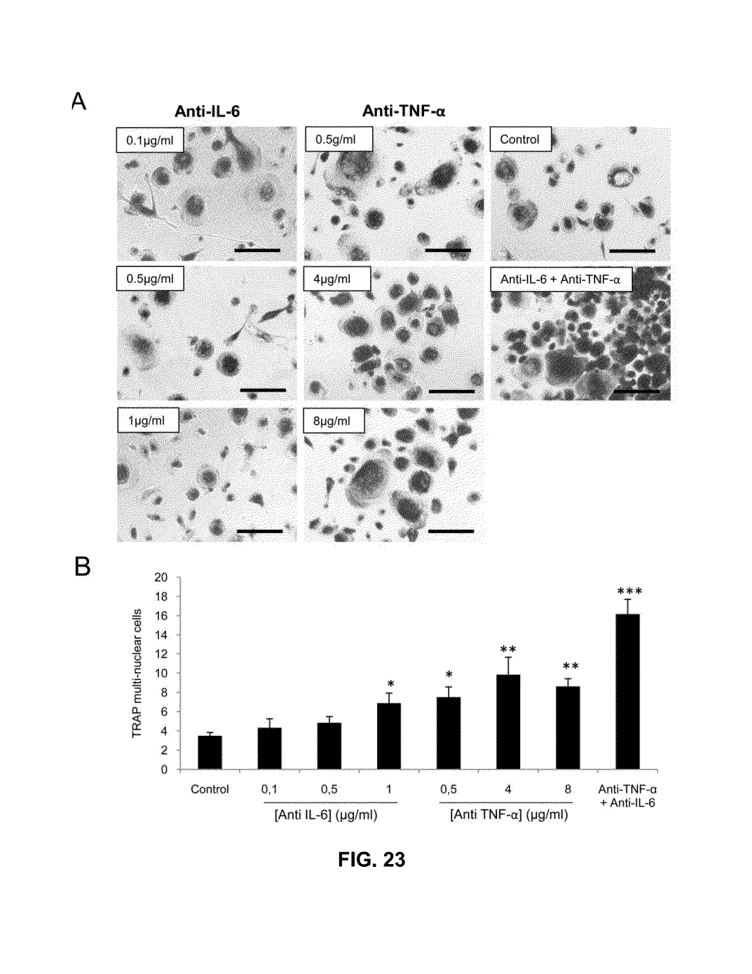

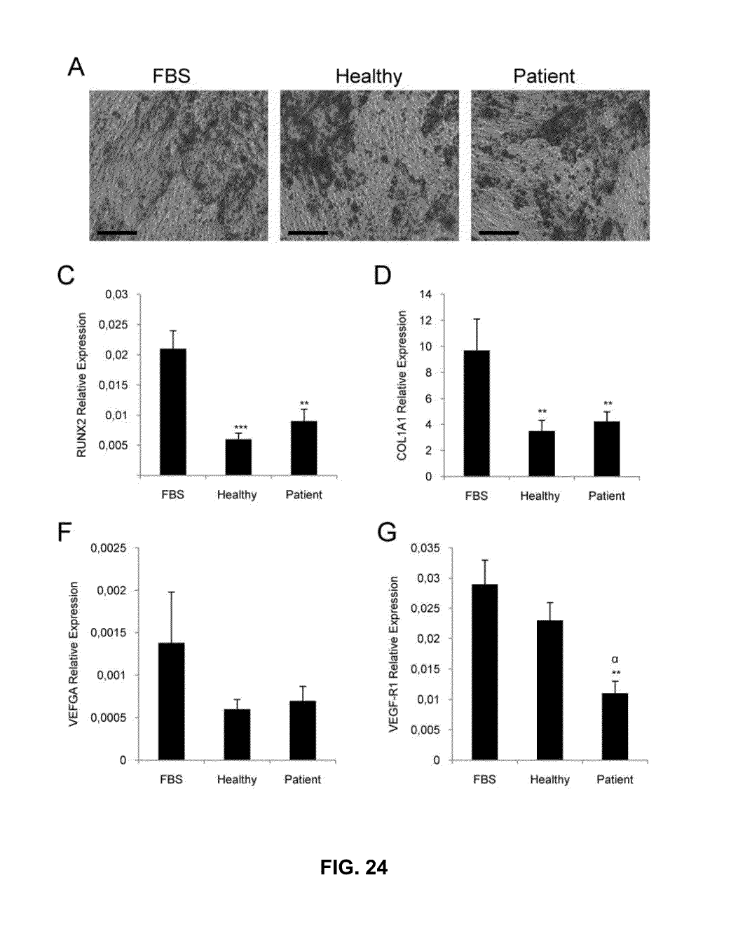

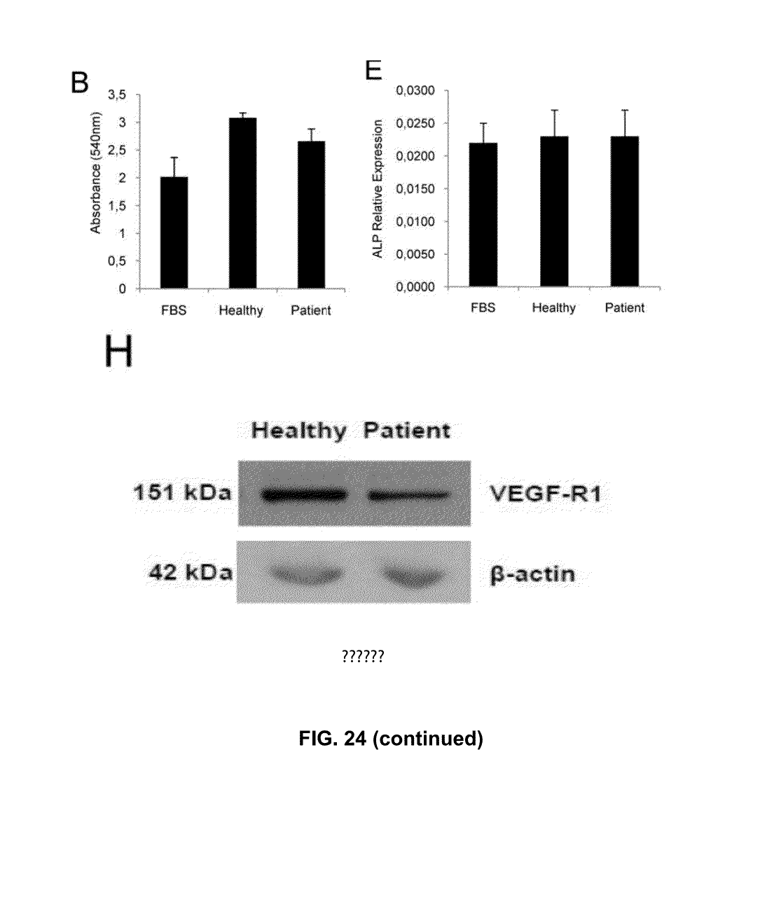

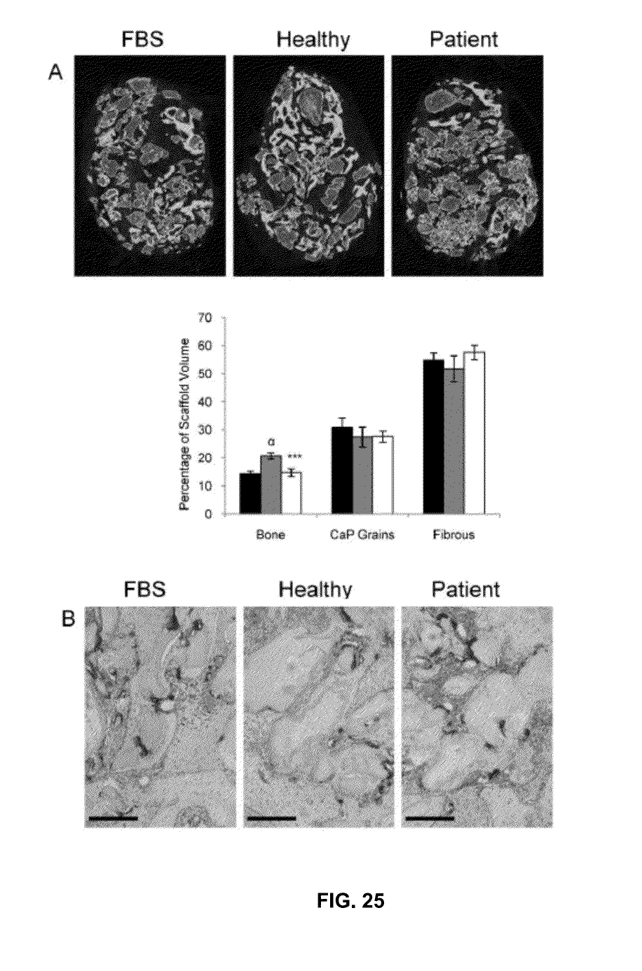

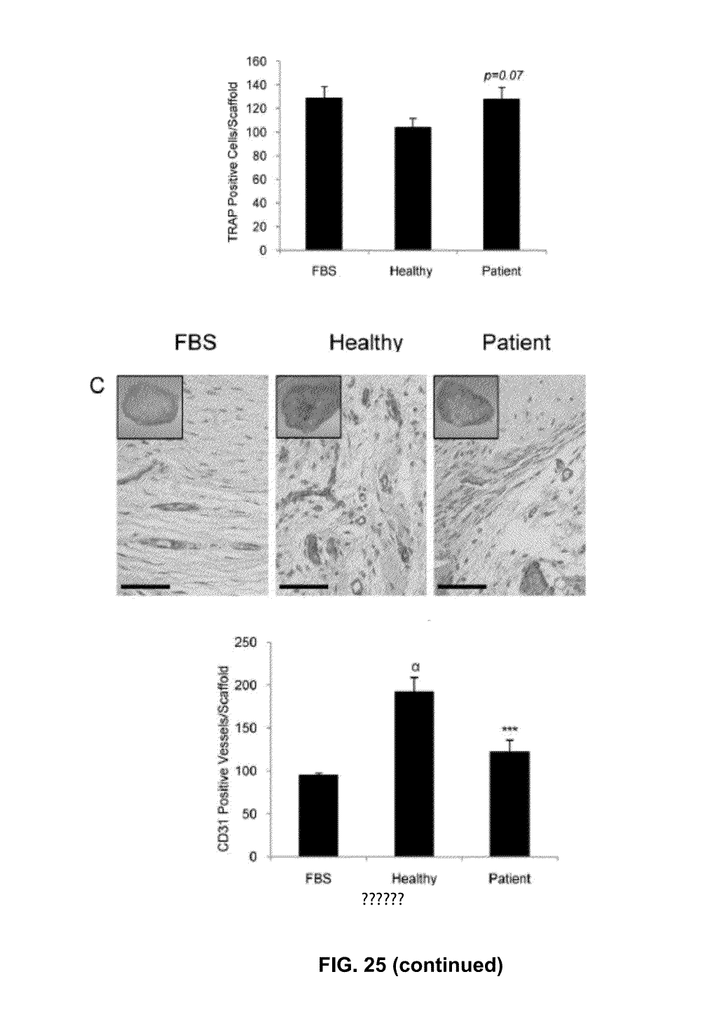

1.-45. (canceled)

46. A method of inhibiting critical illness (MeSH Descriptor: C23.550.291.625) enhanced osteoclastogenesis or increased osteoclast differentiation in a subject diagnosed as suffering from Critical Illness Related Metabolic Bone Disease or critical illness-induced Osteopenia (ICD-10 M85.8, ICD-9 733.90, DiseasesDB 29870 or MeSH D001851) secondary to admission to an intensive care unit (ICU), the method comprising:
administering an autophagy-inducing compound to the subject.

47. The method according to claim 46, wherein the autophagy-inducing compound is an mTOR independent autophagy inducer.

48. The method according to claim 47, wherein the autophagy-inducing compound is used in conjunction with restricting intake of the autophagy-inducing compound by the subject such that autophagy-inducing compound intake is 10 g per day or less, or serum level of the autophagy-inducing compound in the subject is 10 μmol/L or less.

49. The method according to claim 47, wherein the autophagy-inducing compound is used in conjunction with restricting intake of the autophagy-inducing compound by the subject such that autophagy-inducing compound intake is 1 g per day or less, or serum level of the autophagy-inducing compound in the subject is 1000 nmol/L or less.

50. The method according claim 47, wherein the autophagy-inducing compound is a spermidine derivative.

51. The method according to claim 47, wherein the autophagy-inducing compound is a phenothiazine derivative.

52. The method according to claim 51, wherein the autophagy-inducing compound is used in conjunction with restricting intake of the autophagy-inducing compound by the subject such that autophagy-inducing compound intake is 1 g per day or less, or serum level of the autophagy-inducing compound in the subject is 500 nmol/L or less.

53. The method according to claim 47, wherein the autophagy-inducing compound is spermidine.

54. The method according to claim 47, wherein the autophagy-inducing compound is promethazine.

55. The method according to claim 54, wherein the autophagy-inducing compound is used in conjunction with restricting intake of the autophagy-inducing compound by the subject such that autophagy-inducing compound intake is 100 mg per day or less, or serum level of the autophagy-inducing compound in the subject is 150 nmol/L or less.

56. The method according to claim 46, wherein the compound is an mTOR dependent autophagy inducer.

57. The method according to claim 56, wherein the autophagy-inducing compound is used in conjunction with restricting intake of the autophagy-inducing compound by the subject such that autophagy-inducing compound intake is 10 g per day or less, or serum level of the autophagy-inducing compound in the subject is 1000 nmol/L or less.

58. The method according to claim 56, wherein the autophagy-inducing compound is used in conjunction with restricting intake of the autophagy-inducing compound by the subject such that autophagy-inducing compound intake is 1 g per day or less, or serum level of the autophagy-inducing compound in the subject is 1000 nmol/L or less.

59. The method according to claim 56, wherein the mTOR dependent autophagy inducer is rapamycin or a rapamycin derivative.

60. The method according to claim 59, wherein the autophagy-inducing compound is used in conjunction with restricting intake of the autophagy-inducing compound by the subject such that autophagy-inducing compound intake is 5 mg per day or less, or serum level of the autophagy-inducing compound in the subject is 100 nmol/L or less.

61. The method according to claim 59, wherein the autophagy-inducing compound is used in conjunction with restricting intake of the autophagy-inducing compound by the subject such that autophagy-inducing compound intake is 2 mg per day or less, or serum level of the autophagy-inducing compound in the subject is 50 nmol/L or less.

62. The method according to claim 59, wherein the autophagy-inducing compound is used in conjunction with restricting intake of the autophagy-inducing compound by the subject such that autophagy-inducing compound intake is such that serum level of the autophagy-inducing compound in the subject is 10 nmol/L or less.

63. The method according to claim 56, wherein the mTOR dependent autophagy inducer is Everolimus.

64. The method according to claim 56, wherein the mTOR dependent autophagy inducer is Everolimus and wherein the autophagy-inducing compound is used in conjunction with restricting intake of the autophagy-inducing compound by the subject such that autophagy-inducing compound intake is 10 mg per day or less, or serum level of the autophagy-inducing compound in the subject is 100 nmol/L or less.

65. The method according to claim 56, wherein the mTOR dependent autophagy inducer is Everolimus and wherein the autophagy-inducing compound is used in conjunction with restricting intake of the autophagy-inducing compound by the subject such that autophagy-inducing compound intake is 1 mg per day or less, or serum level of the autophagy-inducing compound in the subject is 50 nmol/L or less.

66. The method according to claim 56, wherein the mTOR dependent autophagy inducer is Everolimus and wherein the autophagy-inducing compound is used in conjunction with restricting intake of the autophagy-inducing compound by the subject such that autophagy-inducing compound intake is such that serum level of the autophagy-inducing compound in the subject is 5 nmol/L or less.

67. A method of inhibiting critical illness (MeSH Descriptor: C23.550.291.625)-enhanced osteoclastogenesis or -increased osteoclast differentiation in a subject diagnosed as suffering from Critical Illness Related Metabolic Bone Disease or critical illness-induced Osteopenia (ICD-10 M85.8, ICD-9 733.90, DiseasesDB 29870 or MeSH D001851) secondary to admission to an intensive care unit (ICU), the method comprising:
inhibiting osteoclast formation or osteoclastogenesis in the subject by administering an autophagy-inducing or activating compound.

68. The method according to claim 67, wherein the autophagy-inducing or activating compound is an mTOR independent autophagy inducer.

69. The method according to claim 68, wherein the autophagy-inducing or activating compound is used in conjunction with restricting intake of the autophagy-inducing or activating compound by the subject such that intake of the autophagy-inducing or activating compound is 10 g per day or less or serum level of the autophagy-inducing or activating compound in the subject is 1000 nmol/L or less.

70. The method according to claim 68, wherein the autophagy-inducing or activating compound is used in conjunction with restricting intake of the autophagy-inducing or activating compound by the subject such that intake of the autophagy-inducing or activating compound is 1 g per day or less, or serum level of the autophagy-inducing compound in the subject is 1000 nmol/L or less.

71. The method according to claim 68, wherein the autophagy-inducing or activating compound is a spermidine derivative.

72. The method according to claim 68, wherein the autophagy-inducing or activating compound is a phenothiazine derivative.

73. The method according to claim 72, wherein the autophagy-inducing or activating compound is used in conjunction with restricting intake of the autophagy-inducing or activating compound by the subject such that intake of the autophagy-inducing or activating compound is 1 g per day or less or serum level of the autophagy-inducing compound in the subject is 500 nmol/L or less.

74. The method according to claim 68, wherein the autophagy-inducing or activating compound is spermidine.

75. The method according to claim 68, wherein the autophagy-inducing or activating compound is promethazine.

76. The method according to claim 75, wherein the autophagy-inducing or activating compound is used in conjunction with restricting intake of the autophagy-inducing or activating compound by the subject such that intake of the autophagy-inducing or activating compound is 100 mg per day or less or serum level of the autophagy-inducing compound in the subject is 150 nmol/L or less.

77. The method according to claim 67, wherein the autophagy-inducing or activating compound is an mTOR dependent autophagy inducer.

78. The method according to claim 77, wherein the autophagy-inducing or activating compound is used in conjunction with restricting intake of the autophagy-inducing or activating compound by the subject such that intake of the autophagy-inducing or activating compound is 1 g per day or less or serum level of the autophagy-inducing or activating compound in the subject is 1000 nmol/L or less.

79. The method according to claim 78, wherein the mTOR dependent autophagy inducer is rapamycin or a rapamycin derivative.

80. The method according to claim 79, wherein the autophagy-inducing compound is used in conjunction with restricting intake of the autophagy-inducing or activating compound by the subject such that intake of the autophagy-inducing or activating compound intake is 5 mg per day or less or serum level of the autophagy-inducing or activating compound in the subject is 100 nmol/L or less.

81. The method according to claim 79, wherein the autophagy-inducing or activating compound is used in conjunction with restricting intake of the autophagy-inducing or activating compound by the subject such that intake of the autophagy-inducing or activating compound intake is 2 mg per day or less or serum level of the autophagy-inducing or activating compound in the subject is 50 nmol/L or less.

82. The method according to claim 78, wherein the mTOR dependent autophagy inducer is Everolimus.

83. The method according to claim 82, wherein the autophagy-inducing compound is used in conjunction with restricting intake of the autophagy-inducing or activating compound by the subject such that intake of the autophagy-inducing or activating compound intake is 10 mg per day or less or serum level of the autophagy-inducing or activating compound in the subject is 100 nmol/L or less.

84. The method according to claim 82, wherein the autophagy-inducing compound is used in conjunction with restricting intake of the autophagy-inducing or activating compound by the subject such that intake of the autophagy-inducing or activating compound is 1 mg per day or less or serum level of the autophagy-inducing or activating compound in the subject is 50 nmol/L or less.

85. The method according to claim 67, wherein the subject undergoes total parenteral nutrition.

86. A method of inhibiting for suppressing osteoclastogenesis or osteoclast differentiation in a subject diagnosed as suffering from a disorder of bone density (ICD-10-CM M80-M85), the method comprising:
administering a DNA methylation inhibitor to the subject.

87. The method according to claim 86, wherein enhanced osteoclastogenesis or increased osteoclast differentiation in the subject is suppressed.

88. The method according to claim 86, wherein the subject is suffering from critical illness (MeSH Descriptor: C23.550.291.625), Critical Illness Related Metabolic Bone Disease, or critical illness-induced Osteopenia (ICD-10 M85.8, ICD-9 733.90, DiseasesDB 29870 or MeSH D001851) secondary to admission to an intensive care unit (ICU).

89. The method according to claim 86, wherein the DNA methylation inhibitor is selected from the group consisting of decitabine, 5-aza-2′-deoxycytidine, 5-azadC, azacitidine, 5-azacytidine, vorinostat, procainamide, and a derivative of any thereof.

90. The method according to claim 86, wherein the DNA methylation inhibitor is administered to the subject so as to reach a very low nanomolar plasma concentration of a value in the range of 1 to 10 nM so as to suppress osteoclastogenesis or osteoclast differentiation.


SEQUENCE LISTING (10 sequences)

SEQ ID NO: 1 - DNA, 20 bp, Artificial Sequence
  Human
cgcattcctcatcccagtat

SEQ ID NO: 2 - DNA, 20 bp, Artificial Sequence
  Human
gcctggggtctgtaatctga

SEQ ID NO: 3 - DNA, 20 bp, Artificial Sequence
  Human
gacgaagacatcccaccaat

SEQ ID NO: 4 - DNA, 20 bp, Artificial Sequence
  Human
agatcacgtcatcgcacaac

SEQ ID NO: 5 - DNA, 21 bp, Artificial Sequence
  Human
ggacatgcagtacgtagctga

SEQ ID NO: 6 - DNA, 20 bp, Artificial Sequence
  Human
gtcaattctgcctccttcca

SEQ ID NO: 7 - DNA, 20 bp, Artificial Sequence
  Human
cccactgaggagtccaacat

SEQ ID NO: 8 - DNA, 21 bp, Artificial Sequence
  Human
gcattcacatttgttgtgctg

SEQ ID NO: 9 - DNA, 20 bp, Artificial Sequence
  Human
aagcaaaccacactggcttc

SEQ ID NO: 10 - DNA, 21 bp, Artificial Sequence
  Human
cggggatttcactgtacatct